(19) United States
(12) Patent Application Publication
(10) Pub. No.: US 2005/0202437 A1
(43) Pub. Date: Sep. 15, 2005

(54) Polynucleotides and polypeptides, materials incorporating them and methods for using them

(75) Inventors: Matthew Glenn, Auckland (NZ); Ilkka Havukkala, Auckland (NZ); Mark William Lubbers, Palmerston North (NZ); James Dekker, Palmerston North (NZ)

Correspondence Address: SPECKMAN LAW GROUP PLLC, 1501 WESTERN AVE, SEATTLE, WA, 98101, US

(73) Assignees: GENESIS RESEARCH AND DEVELOPMENT CORPORATION LIMITED, Parnell (NZ); VIALACTIA BIOSCIENCES (NZ) LIMITED, Auckland (NZ)

(21) Appl. No.: 10/650,274

(22) Filed: Aug. 28, 2003

Related U.S. Application Data
(60) Provisional application No. 60/406,810, filed on Aug. 28, 2002.

(51) Int. Cl. C12Q 1/68; C07H 21/04; C12N 9/00; C12N 9/02; C12N 1/21; C12N 15/74

(52) U.S. Cl. 435/6; 435/69.1; 435/183; 435/252.3; 435/320.1; 536/23.2; 435/189

(57) ABSTRACT

Novel polynucleotides isolated from Lactobacillus rhamnosus, as well as oligonucleotide probes and primers, genetic constructs comprising the polynucleotides, biological materials, including plants, microorganisms and multicellular organisms incorporating the polynucleotides, polypeptides expressed by the polynucleotides, and methods for using the polynucleotides and polypeptides are disclosed.

### CROSS-REFERENCE TO RELATED APPLICATIONS

This application claims priority to U.S. Patent Application No. 60/406,810, filed Aug. 28, 2002.

### TECHNICAL FIELD OF THE INVENTION

This invention relates to polynucleotides isolated from lactic acid bacteria as well as to probes and primers specific to the polynucleotides; genetic constructs comprising the polynucleotides; biological materials, including plants, microorganisms and multicellular organisms, incorporating the polynucleotides; polypeptides expressed by the polynucleotides; and methods for using the polynucleotides and polypeptides.

### BACKGROUND OF THE INVENTION

The present invention relates to polynucleotides isolated from a specific strain of lactic acid bacteria, namely Lactobacillus rhamnosus HN001 (L. rhamnosus HN001). Lactic acid bacteria, and their enzymes, are the major determinants of flavor and fermentation characteristics in fermented dairy products, such as cheese and yogurt. Flavors are produced through the action of bacteria and their enzymes on proteins, carbohydrates and lipids.

Lactobacillus rhamnosus strain HN001 are heterofermentative bacteria that are Gram positive, non-motile, non-spore forming, catalase negative, facultative anaerobic rods exhibiting an optimal growth temperature of 37±1° C. and an optimum pH of 6.0-6.5. Experimental studies demonstrated that dietary supplementation with Lactobacillus rhamnosus strain HN001 induced a sustained enhancement in several aspects of both natural and acquired immunity (See PCT International Publication No. WO 99/10476). In addition, L. rhamnosus HN001, and certain other Gram-positive bacteria can specifically and directly modulate human and animal health (See, for example, Tannock et al., Applied Environ. Microbiol. 66:2578-2588, 2000; Gill et al., Brit. J. Nutrition 83:167-176; Quan Shu et al., Food and Chem. Toxicol. 38:153-161, 2000; Quan Shu et al., Intl. J. Food Microbiol. 56:87-96, 2000; Quan Shu et al., Intl. Dairy J. 9:831-836, 1999; Prasad et al., Intl. Dairy J. 8:993-1002, 1998; Sanders and Huis in't Veld, Antonie van Leeuwenhoek 76:293-315, 1999; Salminen et al., 1998. In: Lactic Acid Bacteria, Salminen S and von Wright A (eds)., Marcel Dekker Inc, New York, Basel, Hong Kong, pp. 211-253; Delcour et al., Antonie van Leeuwenhoek 76:159-184, 1999; Blum et al., Antonie van Leeuwenhoek 76:199-205, 1999; Yasui et al., Antonie van Leeuwenhoek 76:383-389, 1999; Hirayama and Rafter, Antonie van Leeuwenhoek 76:391-394, 1999; Ouwehand, 1998. In: Lactic Acid Bacteria, Salminen S and von Wright A (eds)., Marcel Dekker Inc, New York, Basel, Hong Kong, pp. 139-159; Isolauri et al., S 1998. In: Lactic Acid Bacteria, Salminen S and von Wright A (eds)., Marcel Dekker Inc, New York, Basel, Hong Kong, pp. 255-268; Lichtenstein and Goldin, 1998. In: Lactic Acid Bacteria, Salminen S and von Wright A (eds)., Marcel Dekker Inc, New York, Basel, Hong Kong, pp. 269-277; El-Nezami and Ahokas, 1998. In: Lactic Acid Bacteria, Salminen S and von Wright A (eds)., Marcel Dekker Inc, New York, Basel, Hong Kong, pp. 359-367; Nousianen et al., 1998. In: Lactic Acid Bacteria, Salminen S and von Wright A (eds)., Marcel Dekker Inc, New York, Basel, Hong Kong, pp. 437-473; Meisel and Bockelmann, Antonie van Leeuwenhoek 76:207-215, 1999; Christensen et al., Antonie van Leeuwenhoek 76:217-246, 1999; Dunne et al., Antonie van Leeuwenhoek 76:279-292, 1999). Beneficial health effects attributed to these bacteria include the following:

Increased resistance to enteric pathogens and anti-infection activity, including treatment of rotavirus infection and infantile diarrhea—due to increases in antibody production caused by an adjuvant effect, increased resistance to pathogen colonization; alteration of intestinal conditions, such as pH; and the presence of specific antibacterial substances, such as bacteriocins and organic acids.

Aid in lactose digestion—due to lactose degradation by bacterial lactase enzymes (such as beta-galactosidase) that act in the small intestine.

Anti-cancer (in particular anti-colon cancer) and anti-mutagenesis activities—due to anti-mutagenic activity; alteration of procancerous enzymatic activity of colonic microbes; reduction of the carcinogenic enzymes azoreductase, beta-glucuronidase and nitroreductase in the gut and/or faeces; stimulation of immune function; positive influence on bile salt concentration; and antioxidant effects.

Liver cancer reduction—due to aflatoxin detoxification and inhibition of mould growth.

Reduction of small bowel bacterial overgrowth—due to antibacterial activity; and decrease in toxic metabolite production from overgrowth flora.

Immune system modulation and treatment of autoimmune disorders and allergies—due to enhancement of non-specific and antigen-specific defence against infection and tumors; enhanced mucosal immunity; adjuvant effect in antigen-specific immune responses; and regulation of Th1/Th2 cells and production of cytokines.

Treatment of allergic responses to foods—due to prevention of antigen translocation into blood stream and modulation of allergenic factors in food.

Reduction of blood lipids and prevention of heart disease—due to assimilation of cholesterol by bacteria; hydrolysis of bile salts; and antioxidative effects.

Antihypertensive effect—bacterial protease or peptidase action on milk peptides produces antihypertensive peptides. Cell wall components act as ACE inhibitors

Prevention and treatment of urogenital infections—due to adhesion to urinary and vaginal tract cells resulting in competitive exclusion; and production of antibacterial substances (acids, hydrogen peroxide and biosurfactants).

Treatment of inflammatory bowel disorder and irritable bowel syndrome—due to immuno-modulation; increased resistance to pathogen colonization; alteration of intestinal conditions such as pH; production of specific antibacterial substances such as bacteriocins, organic acids and hydrogen peroxide and biosurfactants; and competitive exclusion.

Modulation of infective endocarditis—due to fibronectin receptor-mediated platelet aggregation associated with Lactobacillus sepsis.

Prevention and treatment of Helicobacter pylori infection—due to competitive colonization and antibacterial effect.

Prevention and treatment of hepatic encephalopathy—due to inhibition and/or exclusion of urease-producing gut flora.

Improved protein and carbohydrate utilisation and conversion—due to production of beneficial products by bacterial action on proteins and carbohydrates.

Other beneficial health effects associated with L. rhamnosus include: improved nutrition; regulation of colonocyte proliferation and differentiation; improved lignan and isoflavone metabolism; reduced mucosal permeability; detoxification of carcinogens and other harmful compounds; relief of constipation and diarrhea; and vitamin synthesis, in particular folate.

Peptidases are enzymes that break the peptide bonds linking the amino group of one amino acid with the carboxy group (acid group) of an adjacent amino acid in a peptide chain. The bonds are broken in a hydrolytic reaction. There is a large family of peptidase enzymes that are defined by their specificity for the particular peptides bonds that they cleave (Barrett A J, Rawlings N D and Woessner J F (Eds.) 1998. Handbook of proteolytic enzymes. Academic Press, London, UK). The two main families are exopeptidases and endopeptidases.

Exopeptidases cleave amino acids from the N- or C-terminus of a peptide chain, releasing free amino acids or short (di- and tri-) peptides. Different types of exopeptidases include:
Aminopeptidases—release a free amino acid from the N-terminus of a peptide chain;
dipeptidyl-peptidase (also known as dipeptidyl-aminopeptidases)—release a dipeptide from the N-terminus of a peptide chain;
tripeptidyl-peptidases (also known as tripeptidyl-aminopeptidases)—release a tripeptide from the N-terminus of a peptide chain);
carboxypeptidases—release a free amino acid from the C-terminus of a peptide chain;
peptidyl-dipeptidase—release a dipeptide from the C-terminus of a peptide chain;
dipeptidases—release two free amino acids from a dipeptide; and
tripeptidases—release a free amino acid and a dipeptide from a tripeptide.

Peptidases are important enzymes in the process of cheese ripening and the development of cheese flavor. The hydrolysis of milk caseins in cheese results in textural changes and the development of cheese flavors. The raft of proteolytic enzymes that cause this hydrolysis come from the lactic acid bacteria that are bound up in the cheese—either starter cultures that grow up during the manufacture of the cheese, or adventitious and adjunct non-starter lactic acid bacteria that grow in the cheese as it ripens (Law Haandrikman, Int. Dairy J. 7:1-11, 1997).

Many other enzymes can also influence dairy product flavor, and functional and textural characteristics, as well as influencing the fermentation characteristics of the bacteria, such as speed of growth, acid production and survival (Urbach, Int. Dairy J 5:877-890, 1995; Johnson and Somkuti, Biotech. Appl. Biochem. 13:196-204, 1991; El Soda and Pandian, J. Dairy Sci. 74:2317-2335, 1991; Fox et al,. In Cheese: chemistry, physics and microbiology. Volume 1, General aspects, 2^{nd} edition, P Fox (ed) Chapman and Hall, London; Christensen et al., Antonie van Leeuwenhoek 76:217-246, 1999; Stingle et al., J. Bacteriol. 20:6354-6360, 1999; Stingle et al., Mol. Microbiol. 32:1287-1295, 1999; Lemoine et al., Appl. Environ. Microbiol. 63:1512-3518, 1997). Enzymes influencing specific characteristics and/or functions include the following:
Lysis of cells. These enzymes are mostly cell wall hydrolases, including amidases; muramidases; lysozymes, including N-acetyl muramidase; muramidase; N-acetylglucosaminidase; and N-acetylmuramoyl-L-alanine amidase. DEAD-box helicase proteins also influence autolysis.
Carbohydrate utilization. Lactose, citrate and diacetyl metabolism, and alcohol metabolism are particularly important. The enzymes involved include beta-galactosidase, lactate dehydrogenase, citrate lyase, citrate permease, 2,3 butanediol dehydrogenase (acetoin reductase), acetolactate decarboxylase, acetolactate synthase, pyruvate decarboxylase, pyruvate formate lyase, diacetyl synthase, diacetyl reductase, alcohol decarboxylase, lactate dehydrogenase, pyruvate dehydrogenase, and aldehyde dehydrogenase.
Lipid degradation, modification or synthesis. Enzymes involved include lipases, esterases, phospholipases, serine hydrolases, desaturases, and linoleate isomerase.
Polysaccharide synthesis. Polysaccharides are important not only for potential immune enhancement and adhesion activity but are important for the texture of fermented dairy products. The enzymes involved are a series of glucosyl transferases, including beta-(1-3) glucosyl transferase, alpha-N acetylgalactosaminyl transferase, phosphogalactosyl transferase, alpha-glycosyl transferase, UDP-N-acetylglucosamine C4 epimerase and UDP-N-acetylglucosamine transferase.
Amino acid degradation. Enzymes include glutamate dehydrogenase, aminotransferases, amino acid decarboxylases, and enzymes involved in sulphur amino acid degradation including cystathione beta-lyase.

Sequencing of the genomes, or portions of the genomes, of numerous organisms, including humans, animals, microorganisms and various plant varieties, has been and is being carried out on a large scale. Polynucleotides identified using sequencing techniques may be partial or full-length genes, and may contain open reading frames, or portions of open reading frames, that encode polypeptides. Putative polypeptides may be identified based on polynucleotide sequences and further characterized. The sequencing data relating to polynucleotides thus represents valuable and useful information.

Polynucleotides and polypeptides may be analyzed for varying degrees of novelty by comparing identified sequences to sequences published in various public domain databases, such as EMBL. Newly identified polynucleotides and corresponding putative polypeptides may also be compared to polynucleotides and polypeptides contained in public domain information to ascertain homology to known polynucleotides and polypeptides. In this way, the degree of similarity, identity or homology of polynucleotides and polypeptides having an unknown function may be determined relative to polynucleotides and polypeptides having known functions.

Information relating to the sequences of isolated polynucleotides may be used in a variety of ways. Specified polynucleotides having a particular sequence may be isolated, or synthesized, for use in in vivo or in vitro experimentation as probes or primers. Alternatively, collections of sequences of isolated polynucleotides may be stored using magnetic or optical storage medium and analyzed or manipulated using computer hardware and software, as well as other types of tools.

### SUMMARY OF THE INVENTION

The present invention provides isolated polynucleotides comprising a sequence selected from the group consisting of: (a) sequences identified in the attached Sequence Listing as SEQ ID NOS: 1-80; (b) variants of those sequences; (c) extended sequences comprising the sequences set out in SEQ ID NOS: 1-80, and their variants; and (d) sequences comprising at least a specified number of contiguous residues of a sequence of SEQ ID NOS: 1-80 (x-mers). Oligonucleotide probes and primers corresponding to the sequences set out in SEQ ID NOS: 1-80, and their variants are also provided. All of these polynucleotides and oligonucleotide probes and primers are collectively referred to herein, as “polynucleotides of the present invention.”

The polynucleotide sequences identified as SEQ ID NOS: 1-80 were derived from a microbial source, namely from fragmented genomic DNA of Lactobacillus rhamnosus, strain HN001, described in PCT International Publication No. WO 99/10476. Lactobacillus rhamnosus strain HN001 are heterofermentative bacteria that are Gram positive, non-motile, non-spore forming, catalase negative, facultative anaerobic rods exhibiting an optimal growth temperature of 37±1° C. and an optimum pH of 6.0-6.5. Experimental studies demonstrated that dietary supplementation with Lactobacillus rhamnosus strain HN001 induced a sustained enhancement in several aspects of both natural and acquired immunity. A biologically pure culture of Lactobacillus rhamnosus strain HN001 was deposited at the Australian Government Analytical Laboratories (AGAL), The New South Wales Regional Laboratory, 1 Suakin Street, Pymble, NSW 2073, Australia, as Deposit No. NM97/09514, dated 18 Aug. 1997.

Certain of the polynucleotide sequences disclosed herein are “partial” sequences in that they do not represent a full-length gene encoding a full-length polypeptide. Such partial sequences may be extended by analyzing and sequencing various DNA libraries using primers and/or probes and well-known hybridization and/or PCR techniques. The partial sequences disclosed herein may thus be extended until an open reading frame encoding a polypeptide, a full-length polynucleotide and/or gene capable of expressing a polypeptide, or another useful portion of the genome is identified. Such extended sequences, including full-length polynucleotides and genes, are described as “corresponding to” a sequence identified as one of the sequences of SEQ ID NOS: 1-80 or a variant thereof, or a portion of one of the sequences of SEQ ID NOS: 1-80 or a variant thereof, when the extended polynucleotide comprises an identified sequence or its variant, or an identified contiguous portion (x-mer) of one of the sequences of SEQ ID NOS: 1-80 or a variant thereof.

The polynucleotides identified as SEQ ID NOS: 1-80 were isolated from Lactobacillus rhamnosus genomic DNA clones and represent sequences that are present in the cells from which the DNA was prepared. The sequence information may be used to identify and isolate, or synthesize, DNA molecules such as promoters, DNA-binding elements, open reading frames or full-length genes, that then can be used as expressible or otherwise functional DNA in transgenic organisms. Similarly, RNA sequences, reverse sequences, complementary sequences, antisense sequences and the like, corresponding to the polynucleotides of the present invention, may be routinely ascertained and obtained using the polynucleotides identified as SEQ ID NOS: 1-80.

The present invention further provides isolated polypeptides encoded, or partially encoded, by the polynucleotides disclosed herein. In certain specific embodiments, the polypeptides of the present invention comprise a sequence selected from the group consisting of sequences identified as SEQ ID NO: 81-183, and variants thereof. Polypeptides encoded by the polynucleotides of the present invention may be expressed and used in various assays to determine their biological activity. Such polypeptides may be used to raise antibodies, to isolate corresponding interacting proteins or other compounds, and to quantitatively determine levels of interacting proteins or other compounds.

Genetic constructs comprising the inventive polynucleotides are also provided, together with transgenic host cells comprising such constructs and transgenic organisms, such as microbes, comprising such cells.

The present invention also contemplates methods for modulating the polynucleotide and/or polypeptide content and composition of an organism, such methods involving stably incorporating into the genome of the organism a genetic construct comprising a polynucleotide of the present invention. In one embodiment, the target organism is a microbe, preferably a microbe used in fermentation, more preferably a microbe of the genus Lactobacillus, and most preferably Lactobacillus rhamnosus, or other closely microbial related species used in the dairy industry. In a related aspect, methods for producing a microbe having an altered genotype and/or phenotype is provided, such methods comprising transforming a microbial cell with a genetic construct of the present invention to provide a transgenic cell, and cultivating the transgenic cell under conditions conducive to growth and multiplication. Organisms having an altered genotype or phenotype as a result of modulation of the level or content of a polynucleotide or polypeptide of the present invention compared to a wild-type organism, as well as components and progeny of such organisms, are contemplated by and encompassed within the present invention.

The isolated polynucleotides of the present invention may be usefully employed for the detection of lactic acid bacteria, preferably L. rhamnosus, in a sample material, using techniques well known in the art, such as polymerase chain reaction (PCR) and DNA hybridization, as detailed below.

The inventive polynucleotides and polypeptides may also be employed in methods for the selection and production of more effective probiotic bacteria; as “bioactive” (health-promoting) ingredients and health supplements for immune function enhancement; for reduction of blood lipids such as cholesterol; for production of bioactive material from genetically modified bacteria; as adjuvants; for wound healing; in vaccine development, particularly mucosal vaccines; as animal probiotics for improved animal health and productivity; in selection and production of genetically modified rumen microorganisms for improved animal nutrition and productivity, better flavor and improved milk composition; in methods for the selection and production of better natural food bacteria for improved flavor, faster flavor development, better fermentation characteristics, vitamin synthesis and improved textural characteristics; for the production of improved food bacteria through genetic modification; and for the identification of novel enzymes for the production of, for example, flavors or aroma concentrates.

The isolated polynucleotides of the present invention also have utility in genome mapping, in physical mapping, and in positional cloning of genes of more or less related microbes. Additionally, the polynucleotide sequences identified as SEQ ID NOS: 1-80, and their variants, may be used to design oligonucleotide probes and primers. Such oligonucleotide probes and primers have sequences that are substantially complementary to the polynucleotide of interest over a certain portion of the polynucleotide. Oligonucleotide probes designed using the polynucleotides of the present invention may be used to detect the presence and examine the expression patterns of genes in any organism having sufficiently similar DNA and RNA sequences in their cells, using techniques that are well known in the art, such as slot blot DNA hybridization techniques. Oligonucleotide primers designed using the polynucleotides of the present invention may be used for polymerase chain reaction (PCR) amplifications. Oligonucleotide probes and primers designed using the polynucleotides of the present invention may also be used in connection with various microarray technologies, including the microarray technology of Affymetrix (Santa Clara, Calif.).

The polynucleotides of the present invention may also be used to tag or identify an organism or derived material or product therefrom. Such tagging may be accomplished, for example, by stably introducing a non-disruptive non-functional heterologous polynucleotide identifier into an organism, the polynucleotide comprising at least a portion of a polynucleotide of the present invention.

The polynucleotides of the present invention may also be used as promoters, gene regulators, origins of DNA replication, secretion signals, cell wall or membrane anchors for genetic tools (such as expression or integration vectors).

All references cited herein, including patent references and non-patent publications, are hereby incorporated by reference in their entireties.

### BRIEF DESCRIPTION OF THE DRAWINGS

FIG. 1 shows the nucleotide sequence for L. rhamnosus strain HN001 deoD purine nucleoside phosphorylase AQ1 (SEQ ID NO: 78), showing ATG initiation and translation stop codons (boxed).

FIG. 2 shows the amino acid sequence for L. rhamnosus strain HN001 deoD purine nucleoside phosphorylase AQ1 (SEQ ID NO: 181).

FIG. 3 shows the results of UV light exposure assay measuring relative viability in response to increasing doses of UV light for AQ1⁻ HN001 strain (♦) and wild-type HN001 (▪). Results indicate that the AQ1⁻ HN001 mutant strain showed enhanced survival to exposure to UV light compared to wild-type HN001.

FIG. 4 shows the nucleotide sequence for L. rhamnosus strain HN001 relA GTP pyrophosphokinase gene AM1 (SEQ ID NO: 79) showing ATG initiation and translation stop codons (boxed).

FIG. 5 shows the amino acid sequence of L. rhamnosus strain HN001 relA GTP pyrophosphokinase gene AM1 (SEQ ID NO: 182).

FIG. 6 shows the results of UV light exposure assay measuring relative viability in response to increasing doses of UV light in AM1⁻ HN001 strain (♦) and wild-type HN001 (▪). Results indicate that the AM1⁻ HN001 mutant strain showed enhanced survival to exposure to UV light compared to wild-type HN001.

### DETAILED DESCRIPTION

The polynucleotides disclosed herein were isolated by high throughput sequencing of DNA libraries from the lactic acid bacteria Lactobacillus rhamnosus as described in Example 1. Cell wall, cell surface and secreted components of lactic acid bacteria are known to mediate immune modulation, cell adhesion and antibacterial activities, resulting in many beneficial effects including: resistance to enteric pathogens; modulation of cancer, including colon cancer; anti-mutagenesis effects; reduction of small bowel bacterial overgrowth; modulation of auto-immune disorders; reduction in allergic disorders; modulation of urogenital infections, inflammatory bowel disorder, irritable bowel syndrome, Helicobacter pylori infection and hepatic encephalopathy; reduction of infection with pathogens; regulation of colonocyte proliferation and differentiation; reduction of mucosal permeability; and relief of constipation and diarrhea. These cell components include, but are not limited to, peptidoglycans, teichoic acids, lipoteichoic acids, polysaccharides, adhesion proteins, secreted proteins, surface layer or S-layer proteins, collagen binding proteins and other cell surface proteins, and antibacterial substances such as bacteriocins and organic acids produced by these bacteria. Polynucleotides involved in the synthesis of these proteins and in the synthesis, modification, regulation, transport, synthesis and/or accumulation of precursor molecules for these proteins can be used to modulate the immune effects, antibacterial, cell adhesion and competitive exclusion effects of the bacteria or of components that might be produced by these bacteria.

In order to function effectively as probiotic bacteria, L. rhamnosus HN001 must survive environmental stress conditions in the gastrointestinal tract, as well as commercial and industrial processes. Modification of particular polynucleotides or regulatory processes has been shown to be effective against a number of stresses including oxidative stress, pH, osmotic stress, dehydration, carbon starvation, phosphate starvation, nitrogen starvation, amino acid starvation, heat or cold shock and mutagenic stress. Polynucleotides involved in stress resistance often confer multistress resistance, i.e., when exposed to one stress, surviving cells are resistant to several non-related stresses. Bacterial genes and/or processes shown to be involved in multistress resistance include:

Intracellular phosphate pools—inorganic phosphate starvation leads to the induction of pho regulon genes, and is linked to the bacterial stringent response. Gene knockouts involving phosphate receptor genes appear to lead to multistress resistance.

Intracellular guanosine pools—purine biosynthesis and scavenger pathways involve the production of phosphate-guanosine compounds that act as signal molecules in the bacterial stringent response. Gene knockouts involving purine scavenger pathway genes appear to confer multistress resistance.

Osmoregulatory molecules—small choline-based molecules, such as glycine-betaine, and sugars, such as trehalose, are protective against osmotic shock and are rapidly imported and/or synthesized in response to increasing osmolarity.

Acid resistance—lactobacilli naturally acidify their environment through the excretion of lactic acid, mainly through the cit operon genes responsible for citrate uptake and utilization.

Stress response genes—a number of genes appear to be induced or repressed by heat shock, cold shock, and increasing salt through the action of specific promoters.

The isolated polynucleotides of the present invention, and genetic constructs comprising such polynucleotides, may be employed to produce bacteria having desired phenotypes, including increased resistance to stress and improved fermentation properties.

Many enzymes are known to influence dairy product flavor, functional and textural characteristics as well as general fermentation characteristics such as speed of growth, acid production and survival. These enzymes include those involved in the metabolism of lipids, polysaccharides, amino acids and carbohydrates as well as those involved in the lysis of the bacterial cells.

The isolated polynucleotides and polypeptides of the present invention have demonstrated similarity to polynucleotides and/or polypeptides of known function. The identity and functions of the inventive polynucleotides based on such similarities are shown below in Table 1.

TABLE 1

| SEQ | SEQ ID | | |
|---|---|---|---|
| ID NO: | NO: | | |
| DNA | Polypeptide | Utility | Description |
| 1, 8, 53 | 81, 88, 145 | Removal of undesirable flavor | Homologue of lacG that encodes 6- |
| | | characteristics. | phospho-beta-galactosidase (EC |
| | | Production of desirable flavors. | 3.2.1.85). LacG is part of the lactose |
| | | Modified flavor, aroma, or | metabolism, and hydrolyzes |
| | | texture attributes. | phospholactose, the product of a |
| | | Construction of genetic vectors | phosphor-enolpyruvate-dependent |
| | | for controlled expression of RNA | phosphotransferase system. It belongs |
| | | and/or protein, fusion protein | to the glycosidase family 1 and |
| | | production, genetic modification, | contributes to flavor, including bitter |
| | | mutagenesis amplification of | flavor. |
| | | genetic material or for other | |
| | | genetic or protein manipulations. | |
| | | Altered survival characteristics: | |
| | | (survival of industrial processes, | |
| | | growth or storage in product | |
| | | formats, persistence in gut | |
| | | environment). | |
| | | Modified carbohydrate levels or | |
| | | functional properties. | |
| | | Altered metabolic properties. | |
| | | Modified lactose metabolism. | |
| | | Altered probiotic attributes. | |
| | | Improved fermentation properties | |
| | | or other industrially useful | |
| | | processes. | |
| | | Organisms or materials with | |
| | | improved health properties | |
| | | (including immunoregulatory, | |
| | | anticancer, gut health, lactose | |
| | | tolerance) | |
| 2 | 82 | Production of bioactive or | Homologue of pepS, encoding an |
| | | functional polypeptides. | aminopeptidase (EC 3.4.11.—). PepS |
| | | Removal of undesirable flavor | catalyzes the release of free amino |
| | | characteristics. | acids from peptides. |
| | | Production of desirable flavors. | Aminopeptidases are exopeptidases |
| | | Modified flavor, aroma and/or | and ubiquitous enzymes, frequently |
| | | texture attributes. | observed in animals, plants and |
| | | Construction of genetic vectors | microorganisms. They are involved in |
| | | for controlled expression of RNA | many different functions in the cell, |
| | | and/or protein, fusion protein | such as protein maturation, protein |
| | | production, genetic modification, | turnover, hydrolysis of regulatory |
| | | mutagenesis amplification of | peptides, nitrogen nutrition, |
| | | genetic material or for other | modulation of gene expression etc. |
| | | genetic or protein manipulations. | and, consequently, are considered |
| | | Altered survival characteristics: | essential enzymes. The proteolytic |
| | | survival of industrial processes, | system of lactic acid bacteria is |
| | | growth or storage in product | essential for bacterial growth in milk |
| | | formats, persistence in gut | but also for the development of the |
| | | environment. | organoleptic properties of dairy |
| | | Altered metabolic properties or | products. PepS is involved both in |
| | | regulation of metabolic | bacterial growth by supplying amino |
| | | pathways. | acids, and in the development of |
| | | Altered probiotic attributes. | flavor in dairy products, by |
| | | Organisms or materials with | hydrolyzing peptides (including bitter |
| | | improved health properties | peptides) and liberating aromatic |
| | | (including immunoregulatory, | amino acids which are important |
| | | anticancer, gut health). | precursors of aroma compounds |
| | | Altered resistance to antibiotics. | (Fernandez-Espla and Rul, Eur. J. |
| | | | Biochem. 263: 502-510, 1999). |
| 3 | 83 | Production of bioactive or | Homologue of PepC, encoding |
| | | functional polypeptides. | aminopeptidase C (EC 3.4.22.40). |
| | | Removal of undesirable flavor | PepC is also known as bleomycin |
| | | characteristics. | hydrolase, which inactivates |
| | | Production of desirable flavors. | bleomycin B2 (a cytotoxic |
| | | Modified flavor, aroma and/or | glycometallopeptide) by hydrolysis of |
| | | texture attributes. | a carboxyamide bond of b- |
| | | Construction of genetic vectors | aminoalanine. It also has general |
| | | for controlled expression of RNA | aminopeptidase activity. PepC |
| | | and/or protein, fusion protein | belongs to peptidase family C1; also |
| | | production, genetic modification, | known as the papain family of thiol |
| | | mutagenesis amplification of | proteases and is involved in flavor |
| | | genetic material or for other | production. The proteolytic system of |
| | | genetic or protein manipulations. | lactic acid bacteria is essential for |
| | | Altered survival characteristics: | bacterial growth in milk but also for |
| | | survival of industrial processes, | the development of the organoleptic |
| | | growth or storage in product | properties of dairy products. |
| | | formats, persistence in gut | |
| | | environment. | |
| | | Altered metabolic properties or | |
| | | regulation of metabolic | |
| | | pathways. | |
| | | Altered probiotic attributes. | |
| | | Organisms or materials with | |
| | | improved health properties | |
| | | (including immunoregulatory, | |
| | | anticancer, gut health). | |
| | | Altered resistance to antibiotics. | |
| 4 | 84 | Altered amino acid metabolism. | Homologue of hisC, coding for a L- |
| | | Removal of undesirable flavor | histidinol phosphate aminotransferase |
| | | characteristics. | (EC 2.6.1.9). The HisC |
| | | Production of desirable flavors. | aminotransferase is pyridoxal-5′- |
| | | Modified flavor, aroma and/or | phosphate (PLP)-dependent and is |
| | | texture attributes. | involved in the synthesis of histidine |
| | | Construction of genetic vectors | in eubacteria. HisC also has tyrosine |
| | | for controlled expression of RNA | and phenylalanine aminotransferase |
| | | and/or protein, fusion protein | activity. The biosynthesis of histidine |
| | | production, genetic modification, | is a central metabolic process in |
| | | mutagenesis amplification of | organisms ranging from bacteria to |
| | | genetic material or for other | yeast and plants. |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved fermentation properties | |
| | | or other industrially useful | |
| | | processes. | |
| 5 | 85 | Production of desirable flavors. | Homologue of mvaD coding for |
| | | Modified flavor, aroma and/or | mevalonate pyrophosphate |
| | | texture attributes. | decarboxylase (EC 4.1.1.33). MvaD |
| | | Construction of genetic vectors | is part of the mevalonate pathway for |
| | | for controlled expression of RNA | the biosynthesis of the central |
| | | and/or protein, fusion protein | isoprenoid precursor, isopentenyl |
| | | production, genetic modification, | diphosphate by catalyzing the |
| | | mutagenesis amplification of | reaction of mevalonate 5-diphosphate |
| | | genetic material or for other | with ATP to produce inorganic |
| | | genetic or protein manipulations. | phosphate, ADP, CO₂ and |
| | | Altered survival characteristics: | isopentenyl diphosphate, the building |
| | | survival of industrial processes, | block of sterol and isoprenoid |
| | | growth or storage in product | biosynthesis. |
| | | formats, persistence in gut | |
| | | environment. | |
| | | Altered metabolic properties. | |
| | | Modified carbohydrate levels or | |
| | | functional properties. | |
| | | Altered probiotic attributes. | |
| | | Organisms or materials with | |
| | | improved health properties | |
| | | (including immunoregulatory, | |
| | | anticancer, gut health). | |
| 6 | 86 | Production of desirable flavors. | Homologue of protein p60, encoded |
| | | Modified flavor, aroma and/or | by the gene termed iap. The p60 |
| | | texture attributes. | protein is a major extracellular |
| | | Construction of genetic vectors | product secreted by all isolates of L. monocytogenes. |
| | | for controlled expression of RNA | This protein has |
| | | and/or protein, fusion protein | peptidoglycan hydrolase activity but |
| | | production, genetic modification, | also influences the uptake of L. monocytogenes |
| | | mutagenesis amplification of | by non-phagocytic |
| | | genetic material or for other | cells. Proteins related to p60 are also |
| | | genetic or protein manipulations. | found in all other Listeria species. It |
| | | Altered survival characteristics: | has been shown that p60 protein is |
| | | survival of industrial processes, | among the strongest antigens in |
| | | growth or storage in product | listeriae for B- and T-cell responses. |
| | | formats, persistence in gut | The protein p60 belongs to the E. coli |
| | | environment. | nlpc/listeria p60 family. |
| | | Altered metabolic properties. | |
| | | Altered probiotic attributes. | |
| | | Modified adhesion to human or | |
| | | animal cells or cell lines. | |
| | | Organisms or materials with | |
| | | improved health properties | |
| | | (including immunoregulatory, | |
| | | anticancer, gut health) | |
| | | Altered resistance to antibiotics. | |
| | | Improved antimicrobial | |
| | | properties. | |
| 7 | 87 | Construction of genetic vectors | Homologue of elongation factors Tu |
| | | for controlled expression of RNA | (EF-Tu) and 1 alpha (EF-1alpha) that |
| | | and/or protein, fusion protein | are homologous proteins essential to |
| | | production, genetic modification, | translation in bacteria and eukaryotes, |
| | | mutagenesis amplification of | respectively. EF-Tu and EF-1alpha |
| | | genetic material or for other | are GTPases that catalyze the binding |
| | | genetic or protein manipulations. | of aminoacyl-tRNAs to the A-site of |
| | | Production of desirable flavors. | the ribosome. As they are among the |
| | | Modified flavor, aroma and/or | slowest evolving proteins known, EFs |
| | | texture attributes. | are used to study cellular functions |
| | | Altered survival characteristics: | and to root the universal tree of life |
| | | survival of industrial processes, | (Gaucher et al. Proc. Natl. Acad. Sci. |
| | | growth or storage in product | USA 98: 548-552, 2001), and are |
| | | formats, persistence in gut | therefore an excellent genetic tool. |
| | | environment. | |
| | | Altered metabolic properties or | |
| | | regulation of metabolic | |
| | | pathways. | |
| | | Altered probiotic attributes. | |
| 9 | 89 | Construction of genetic vectors | Homologue of ribonuclease HII (EC |
| | | for controlled expression of RNA | 3.1.26.4), an RNAse that specifically |
| | | and/or protein, fusion protein | degrades the RNA moiety in |
| | | production, genetic modification, | RNA/DNA hybrids. Endogenous |
| | | mutagenesis amplification of | RNase H activity plays an essential |
| | | genetic material or for other | role in biological effects mediated by |
| | | genetic or protein manipulations. | antisense oligonucleotides, molecules |
| | | Production of desirable flavors. | considered as potential agents against |
| | | Modified flavor, aroma and/or | infectious diseases and pathologies |
| | | texture attributes. | resulting from dysfunctional genes. |
| | | Altered survival characteristics: | The prokaryotic RNAse HII is the |
| | | survival of industrial processes, | evolutionary counterpart of the major |
| | | growth or storage in product | mammalian RNase H (Frank et al., |
| | | formats, persistence in gut | Proc. Natl. Acad. Sci. USA 95: 12872-12877, |
| | | environment. | 1998) and is necessary for cell |
| | | Altered metabolic properties or | survival. |
| | | regulation of metabolic | |
| | | pathways. | |
| | | Altered probiotic attributes. | |
| 10 | 90 | Production of desirable flavors. | Lipase homologue. Lipases are |
| | | Modified flavor, aroma and/or | enzymes that catalyze hydrolysis of |
| | | texture attributes. | fatty acid ester bonds in |
| | | Construction of genetic vectors | triacylglycerol (TAG) and releasing |
| | | for controlled expression of RNA | free fatty acids. The reaction is |
| | | and/or protein, fusion protein | reversible and therefore the enzyme |
| | | production, genetic modification, | can catalyze esterification of glycerol |
| | | mutagenesis amplification of | to form mono, di and triglycerides. |
| | | genetic material or for other | Free fatty acids are important in |
| | | genetic or protein manipulations. | providing flavor-bearing compounds |
| | | Altered survival characteristics: | for dairy products such as cheese, and |
| | | survival of industrial processes, | have a significant role in both flavor |
| | | growth or storage in product | and texture. Used extensively in wide |
| | | formats, persistence in gut | range of convenience foods. Short |
| | | environment. | chain fatty acids are known to have a |
| | | Altered metabolic properties. | variety of health impacts. |
| | | Modified lipid, glycolipid or free | |
| | | fatty acid levels or functional | |
| | | properties. | |
| | | Modified production of short | |
| | | chain fatty acids. | |
| | | Altered lipid metabolism. | |
| | | Altered probiotic attributes. | |
| | | Organisms or materials with | |
| | | improved health properties | |
| | | (including immunoregulatory, | |
| | | anticancer, gut health). | |
| 11 | 91 | Altered amino acid metabolism. | Homologue of hisD, encoding |
| | | Removal of undesirable flavor | histidinol dehydrogenase (HDH, EC |
| | | characteristics. | 1.1.1.23). HisD catalyzes the last two |
| | | Production of desirable flavors. | steps in the biosynthesis of L- |
| | | Modified flavor, aroma and/or | histidine: sequential NAD-dependent |
| | | texture attributes. | oxidations of L-histidinol to L- |
| | | Construction of genetic vectors | histidinaldehyde and then to L- |
| | | for controlled expression of RNA | histidine. Because hisD is absent in |
| | | and/or protein, fusion protein | mammals, it is a target for inhibition |
| | | production, genetic modification, | as part of herbicide development |
| | | mutagenesis amplification of | (Barbosa et al., Proc. Natl. Acad. Sci. |
| | | genetic material or for other | USA 99: 1859-1864, 2002). |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 11 | 92 | Altered amino acid metabolism. | Homologue of hisZ, coding for an |
| | | Removal of undesirable flavor | aminoacyl-tRNA synthetase. HisZ is |
| | | characteristics. | an essential component of the first |
| | | Production of desirable flavors. | enzyme in histidine biosynthesis with |
| | | Modified flavor, aroma and/or | ATP phosphoribosyltransferase |
| | | texture attributes. | (HisG, EC 2.4.2.17) but lacks |
| | | Construction of genetic vectors | aminoacylation activity. HisZ is also |
| | | for controlled expression of RNA | called an ATP |
| | | and/or protein, fusion protein | phosphoribosyltransferase regulatory |
| | | production, genetic modification, | subunit. HisZ is required for histidine |
| | | mutagenesis amplification of | prototrophy and directly involved in |
| | | genetic material or for other | the transferase function. Both HisG |
| | | genetic or protein manipulations. | and HisZ are required for catalyzing |
| | | Altered survival characteristics: | the ATP phosphoribosyltransferase |
| | | survival of industrial processes, | reaction. Aminoacyl-tRNA |
| | | growth or storage in product | synthetases have an essential catalytic |
| | | formats, persistence in gut | role in protein biosynthesis, but also |
| | | environment. | participate in numerous other |
| | | Regulation of amino acid | functions, including regulation of |
| | | metabolism. | gene expression and amino acid |
| | | Altered metabolic properties. | biosynthesis via transamidation |
| | | Altered probiotic attributes. | pathways (Sissler et al., Proc. Natl. |
| | | Modified health properties | Acad. Sci. USA 96: 8985-8990, 1999). |
| | | (including immunoregulatory, | Because HisD is absent in mammals, |
| | | anticancer, gut health). | it is a target for inhibition as part of |
| | | Modified antibiotic resistance. | herbicide development (Barbosa et |
| | | | al., Proc. Natl. Acad. Sci. USA |
| | | | 99: 1859-1864, 2002). |
| 12 | 93 | Altered amino acid metabolism. | Homologue of proA, coding for a |
| | | Removal of undesirable flavor | glutamate-5-semialdehyde |
| | | characteristics. | dehydrogenase (EC 1.2.1.41). ProA is |
| | | Production of desirable flavors. | also known as gamma- |
| | | Modified flavor, aroma and/or | glutamylphosphate reductase, and |
| | | texture attributes. | catalyzes the second step of proline |
| | | Construction of genetic vectors | biosynthesis, the NADPH-dependent |
| | | for controlled expression of RNA | reduction of L-gamma-glutamyl 5- |
| | | and/or protein, fusion protein | phosphate into L-glutamate 5- |
| | | production, genetic modification, | semialdehyde and phosphate. |
| | | mutagenesis amplification of | Intracellular accumulation of the |
| | | genetic material or for other | amino acid proline has been linked to |
| | | genetic or protein manipulations. | salt tolerance and virulence potential |
| | | Altered survival characteristics: | of a number of bacteria. Proline |
| | | survival of industrial processes, | biosynthesis plays an important role |
| | | growth or storage in product | in survival in osmolyte-depleted |
| | | formats, persistence in gut | environments of elevated osmolarity. |
| | | environment. | The survival of the food-borne |
| | | Altered metabolic properties. | pathogen L. monocytogenes in |
| | | Altered probiotic attributes. | hypersaline environments is |
| | | Modified health properties | attributed mainly to the accumulation |
| | | (including immunoregulatory, | of organic compounds termed |
| | | anticancer, gut health). | osmolytes. Osmolytes, often referred |
| | | Modified antibiotic resistance. | to as compatible solutes owing to |
| | | Improved antimicrobial | their compatibility with cellular |
| | | properties. | metabolism at high internal |
| | | | concentrations, can be either |
| | | | transported into the cell or |
| | | | synthesized de novo and act by |
| | | | counterbalancing the external osmotic |
| | | | strength, thus preventing water loss |
| | | | and plasmolysis. As well as its role as |
| | | | an osmoprotectant, proline may |
| | | | function as a virulence factor for |
| | | | certain pathogenic bacteria (Sleator et |
| | | | al., Appl. Environ. Microbiol. |
| | | | 67: 2571-2577, 2001). |
| 12 | 94 | Altered amino acid metabolism. | Homologue of proB, coding for |
| | | Removal of undesirable flavor | gamma-glutamyl kinase (EC |
| | | characteristics. | 2.7.2.11) also known as glutamate 5- |
| | | Production of desirable flavors. | kinase 1. ProB catalyzes the first step |
| | | Modified flavor, aroma and/or | of proline biosynthesis, the transfer of |
| | | texture attributes. | a phosphate group to glutamate to |
| | | Construction of genetic vectors | form glutamate 5-phosphate which |
| | | for controlled expression of RNA | rapidly cyclizes to 5-oxoproline. |
| | | and/or protein, fusion protein | Intracellular accumulation of the |
| | | production, genetic modification, | amino acid proline has been linked to |
| | | mutagenesis amplification of | the salt tolerance and virulence |
| | | genetic material or for other | potential of a number of bacteria. |
| | | genetic or protein manipulations. | Proline biosynthesis plays an |
| | | Altered survival characteristics: | important role in survival in |
| | | survival of industrial processes, | osmolyte-depleted environments of |
| | | growth or storage in product | elevated osmolarity. The survival of |
| | | formats, persistence in gut | the food-borne pathogen L. monocytogenes |
| | | environment. | in hypersaline |
| | | Altered metabolic properties. | environments is attributed mainly to |
| | | Altered probiotic attributes. | the accumulation of organic |
| | | Modified health properties | compounds termed osmolytes. |
| | | (including immunoregulatory, | Osmolytes, often referred to as |
| | | anticancer, gut health). | compatible solutes owing to their |
| | | Modified antibiotic resistance. | compatibility with cellular |
| | | Improved antimicrobial | metabolism at high internal |
| | | properties. | concentrations, can be either |
| | | | transported into the cell or |
| | | | synthesized de novo and act by |
| | | | counterbalancing the external osmotic |
| | | | strength, thus preventing water loss |
| | | | and plasmolysis. As well as its role as |
| | | | an osmoprotectant, proline may |
| | | | function as a virulence factor for |
| | | | certain pathogenic bacteria (Sleator et |
| | | | al., Appl. Environ. Microbiol. |
| | | | 67: 2571-2577, 2001). |
| 13 | 95 | Altered cell wall or cell surface | Homologue of vanHE or LDHD, |
| | | characteristics, structures or | encoding a D-lactate dehydrogenase |
| | | functions. | (D-LDH, EC 1.1.1.28). D-LDH |
| | | Modified adhesion to human or | reduces pyruvate to D-lactate and is |
| | | animal cells or cell lines. | involved in bacterial cell wall |
| | | Production of desirable flavors. | structure and function. VanH plays an |
| | | Modified flavor, aroma and/or | essential role in bacterial resistance to |
| | | texture attributes. | the antibiotic vancomycin. |
| | | Construction of genetic vectors | |
| | | for controlled expression of RNA | |
| | | and/or protein, fusion protein | |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| 14 | 96 | Altered amino acid metabolism. | Homologue of metA that encodes |
| | | Removal of undesirable flavor | homoserine O-transsuccinylase (EC |
| | | characteristics. | 2.3.1.46). MetA catalyzes the first |
| | | Production of desirable flavors. | unique step in bacterial and plant |
| | | Modified flavor, aroma and/or | methionine biosynthesis involving the |
| | | texture attributes. | activation of the gamma-hydroxyl of |
| | | Construction of genetic vectors | homoserine. The activity of this |
| | | for controlled expression of RNA | enzyme is closely regulated in vivo |
| | | and/or protein, fusion protein | and therefore represents a critical |
| | | production, genetic modification, | control point for cell growth and |
| | | mutagenesis amplification of | viability. |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer gut health). | |
| | | Modified antibiotic resistance. | |
| 15 | 97 | Production of desirable flavors. | Homologue of plnG, encoding an |
| | | Modified flavor, aroma and/or | ATP binding cassette (ABC) |
| | | texture attributes. | transporter for the antimicrobial |
| | | Construction of genetic vectors | compound (bacteriocin) plantaricin |
| | | for controlled expression of RNA | A. PlnG displays strong similarities |
| | | and/or protein, fusion protein | to the proposed transport proteins of |
| | | production, genetic modification, | several other bacteriocins and to |
| | | mutagenesis amplification of | proteins implicated in the signal- |
| | | genetic material or for other | sequence-independent export of |
| | | genetic or protein manipulations. | Escherichia coli hemolysin, PlnH is |
| | | Altered survival characteristics: | its accessory protein (Huhne et al., |
| | | survival of industrial processes, | Microbiol. 142: 1437-1448, 1996). |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer gut health). | |
| | | Modified antibiotic resistance. | |
| | | Improved antimicrobial | |
| | | properties. | |
| 16 | 98 | Construction of genetic vectors | Homologue of hemN that encodes an |
| | | for controlled expression of RNA | oxygen-independent |
| | | and/or protein, fusion protein | coproporphyrinogen III |
| | | production, genetic modification, | dehydrogenase (EC 1.3.3.3). HemN |
| | | mutagenesis amplification of | catalyzes the oxidative |
| | | genetic material or for other | decarboxylation of |
| | | genetic or protein manipulations. | coproporphyrinogen III to yield |
| | | Production of desirable flavors. | protoporphyrinogen IX and requires |
| | | Modified flavor, aroma and/or | NADP+, ATP, Mg2+, and L- |
| | | texture attributes. | methionine. In association with |
| | | Altered survival characteristics: | specific apoproteins, it serves a wide |
| | | survival of industrial processes, | range of important functions |
| | | growth or storage in product | including electron transport (e.g., |
| | | formats, persistence in gut | cytochromes), binding and transport |
| | | environment. | of O₂ (e.g., hemoglobin), and |
| | | Altered metabolic properties or | oxidative catalysis (e.g., peroxidases) |
| | | regulation of metabolic | (Fischer et al., J. Bacteriol. |
| | | pathways. | 183: 1300-1311, 2001). |
| | | Altered probiotic attributes. | |
| | | Altered antimicrobial properties. | |
| | | Modified health properties | |
| | | (including immunoregulatory, | |
| | | anticancer gut health). | |
| 17 | 99 | Removal of undesirable flavor | Homologue of lacD, encoding |
| | | characteristics. | tagatose-1,6-bisphosphate aldolase |
| | | Modified flavor, aroma, texture | (EC 4.1.2.40). LacD is responsible |
| | | attributes. | for the aldol cleavage of tagatose-1,6- |
| | | Construction of genetic vectors | bisphosphate to form glycerone-P and |
| | | for controlled expression of RNA | glyceraldehyde 3-phosphate in the |
| | | and/or protein, fusion protein | tagatose 6-phosphate pathway of |
| | | production, genetic modification, | lactose catabolism in bacteria. The |
| | | mutagenesis amplification of | enzyme activity is stimulated by |
| | | genetic material or for other | certain divalent cations. |
| | | genetic or protein manipulations. | |
| | | Altered survival characteristics: | |
| | | (survival of industrial processes, | |
| | | growth or storage in product | |
| | | formats, persistence in gut | |
| | | environment). | |
| | | Modified carbohydrate levels or | |
| | | functional properties. | |
| | | Altered metabolic properties. | |
| | | Modified lactose metabolism. | |
| | | Altered probiotic attributes. | |
| | | Organisms or materials with | |
| | | improved health properties | |
| | | (including immunoregulatory, | |
| | | anticancer, gut health, lactose | |
| | | tolerance). | |
| 18 | 100 | Altered amino acid metabolism. | Homologue of asnH, encoding |
| | | Removal of undesirable flavor | asparagine synthetase [glutamine- |
| | | characteristics. | hydrolyzing] 2 (EC 6.3.5.4). AsnH |
| | | Altered cell wall or cell surface | transamidates asparagine, glutamate, |
| | | characteristics, structures or | AMP and diphosphate from aspartate, |
| | | functions. | glutamine and ATP as part of the |
| | | Production of desirable flavors. | asparagine biosynthesis pathway. |
| | | Modified flavor, aroma and/or | AsnH gene is also involved in cell |
| | | texture attributes. | surface organization. |
| | | Construction of genetic vectors | |
| | | for controlled expression of RNA | |
| | | and/or protein, fusion protein | |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Improved fermentation properties | |
| | | or other industrially useful | |
| | | processes. | |
| 19 | 101 | Removal of undesirable flavor | Homologue of butB, encoding 2,3- |
| | | characteristics. | butanediol dehydrogenase (EC |
| | | Production of desirable flavors. | 1.1.1.4). ButB catalyzes the NAD+- |
| | | Modified flavor, aroma and/or | dependent oxidation of 2,3- |
| | | texture attributes. | butanediol acetoin, as well as the |
| | | Construction of genetic vectors | corresponding reverse reactions. It |
| | | for controlled expression of RNA | can also reduce diacetyl to acetoin. |
| | | and/or protein, fusion protein | Diacetyl is an important flavor |
| | | production, genetic modification, | compound in dairy products. |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved fermentation properties | |
| | | or other industrially useful | |
| | | processes. | |
| 20 | 102 | Altered cell wall or cell surface | Homologue of a peptidoglycan |
| | | characteristics, structures or | hydrolase (N-acetylmuramoyl-L- |
| | | functions. | alanine amidase). N-acetylmuramoyl- |
| | | Modified adhesion to human or | L-alanine amidase is an autolysin |
| | | animal cells or cell lines. | involved in degrading the cell wall |
| | | Production of desirable flavors. | during cell growth or programmed |
| | | Modified flavor, aroma and/or | cell death and is involved in cell |
| | | texture attributes. | growth and important for releasing |
| | | Construction of genetic vectors | enzymes important for flavor. |
| | | for controlled expression of RNA | |
| | | and/or protein, fusion protein | |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved fermentation properties | |
| | | or other industrially useful | |
| | | processes. | |
| 21, 76 | 103, 175 | Altered cell wall or cell surface | Homologue of galC, encoding |
| | | characteristics, structures or | galactosylceramidase (EC 3.2.1.46). |
| | | functions. | GalC hydrolyzes galactose ester |
| | | Modified adhesion to human or | bonds of galactosylceramide, |
| | | animal cells or cell lines. | galactosylsphingosine, |
| | | Production of desirable flavors. | monogalactosyldiglyceride and |
| | | Modified flavor, aroma and/or | lactosylceramide. It is involved in |
| | | texture attributes. | the catabolism of galactosylceramide, |
| | | Construction of genetic vectors | a major lipid in myelin, kidney and |
| | | for controlled expression of RNA | epithelial cells of small intestine and |
| | | and/or protein, fusion protein | colon. While bacteria may use galC |
| | | production, genetic modification, | to release sugars for metabolism, the |
| | | mutagenesis amplification of | by-products, including ceramide, asct |
| | | genetic material or for other | as signalling molecules in eukaryotic |
| | | genetic or protein manipulations. | cells and can lead to apoptosis or |
| | | Altered survival characteristics: | differentiation. Therefore, glaC plays |
| | | survival of industrial processes, | a role in probiotic effects and survival |
| | | growth or storage in product | in the gut environment. |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health, apoptosis). | |
| | | Modified antibiotic resistance. | |
| | | Improved antimicrobial | |
| | | properties. | |
| | | Improved fermentation properties | |
| | | or other industrially useful | |
| | | processes. | |
| 22 | 104 | Production of bioactive or | Homologue of pepN, encoding |
| | | functional polypeptides. | membrane alanyl aminopeptidase |
| | | Removal of undesirable flavor | (EC 3.4.11.2), also called lysyl |
| | | characteristics. | aminopeptidase and aminopeptidase |
| | | Production of desirable flavors. | N. PepN releases the N-terminal |
| | | Modified flavor, aroma and/or | amino acid, Xaa-\|-Xbb- from a |
| | | texture attributes. | peptide, amide or arylamide. |
| | | Construction of genetic vectors | Aminopeptidases are involved in |
| | | for controlled expression of RNA | many different functions in the cell, |
| | | and/or protein, fusion protein | such as protein maturation, protein |
| | | production, genetic modification, | turnover, hydrolysis of regulatory |
| | | mutagenesis amplification of | peptides, nitrogen nutrition, |
| | | genetic material or for other | modulation of gene expression etc. |
| | | genetic or protein manipulations. | and, consequently, are considered |
| | | Altered survival characteristics: | essential enzymes. The proteolytic |
| | | survival of industrial processes, | system of lactic acid bacteria is |
| | | growth or storage in product | essential for bacterial growth in milk |
| | | formats, persistence in gut | but also for the development of the |
| | | environment. | organoleptic properties of dairy |
| | | Altered metabolic properties or | products. PepN is involved both in |
| | | regulation of metabolic | bacterial growth by supplying amino |
| | | pathways. | acids, and in the development of |
| | | Altered probiotic attributes. | flavor in dairy products, by |
| | | Organisms or materials with | hydrolyzing peptides (including bitter |
| | | improved health properties | peptides) and liberating aromatic |
| | | (including immunoregulatory, | amino acids which are important |
| | | anticancer, gut health). | precursors of aroma compounds |
| | | Altered resistance to antibiotics. | (Fernandez-Espla and Rul, Eur. J. |
| | | | Biochem. 263: 502-510, 1999). |
| 23 | 105 | Production of desirable flavors. | Homologue of mvaB, encoding 3- |
| | | Modified flavor, aroma and/or | hydroxy-3-methylglutaryl coenzyme |
| | | texture attributes. | A synthase. MvaB catalyzes the |
| | | Construction of genetic vectors | condensation of acetyl-CoA with |
| | | for controlled expression of RNA | acetoacetyl-CoA to form 3-hydroxy- |
| | | and/or protein, fusion protein | 3-methylglutaryl-CoA and CoA. |
| | | production, genetic modification, | MvaB is involved in mevalonic acid |
| | | mutagenesis amplification of | metabolism as well as in biosynthesis |
| | | genetic material or for other | of cholesterol and ubiquinone |
| | | genetic or protein manipulations. | progenitors. Terpenoids or |
| | | Altered survival characteristics: | isoprenoids constitute a vast family of |
| | | survival of industrial processes, | organic compounds that includes |
| | | growth or storage in product | sterols and carotenoids that have |
| | | formats, persistence in gut | flavor, color, texture and other |
| | | environment. | sensory impacts on food products. |
| | | Altered metabolic properties. | The terpenoids in many organisms |
| | | Modified carbohydrate levels or | share early steps in their biosynthesis, |
| | | functional properties. | including the synthesis of 3-hydroxy- |
| | | Altered probiotic attributes. | 3-methylglutaryl-coenzyme A and its |
| | | Organisms or materials with | conversion to mevalonate. |
| | | improved health properties | |
| | | (including immunoregulatory | |
| | | anticancer, gut health). | |
| 24 | 106 | Construction of genetic vectors | Homologue of hexB, one of two |
| | | for controlled expression of RNA | proteins involved in DNA mismatch |
| | | and/or protein, fusion protein | repair. The hex mismatch repair |
| | | production, genetic modification, | system of Streptococcus pneumoniae |
| | | mutagenesis amplification of | acts both during transformation (a |
| | | genetic material or for other | recombination process that directly |
| | | genetic or protein manipulations. | produces heteroduplex DNA) to |
| | | Production of desirable flavors. | correct donor strands and after DNA |
| | | Modified flavor, aroma and/or | replication to remove |
| | | texture attributes. | misincorporated nucleotides. The |
| | | Altered survival characteristics: | hexB is one of at least two proteins |
| | | survival of industrial processes, | required for mismatch repair. HexB is |
| | | growth or storage in product | homologous to the mutL protein, |
| | | formats, persistence in gut | which is required for methyl-directed |
| | | environment. | mismatch repair in Salmonella |
| | | Altered viability in response to | typhimurium and E. coli, and to the |
| | | stress conditions. | PMS1 gene product, which is likely |
| | | Altered metabolic properties or | to be involved in a mismatch |
| | | regulation of metabolic | correction system in Saccharomyces |
| | | pathways. | cerevisiae (Prudhomme et al., J. Bacteriol. |
| | | Altered probiotic attributes. | 171: 5332-5338, 1989). |
| 25 | 107 | Altered amino acid metabolism. | Homologue of araT, encoding an |
| | | Removal of undesirable flavor | aromatic amino acid aminotransferase |
| | | characteristics. | (EC 2.6.1.57). Aminotransferases |
| | | Production of desirable flavors. | have been widely applied in the |
| | | Modified flavor, aroma and/or | large-scale biosynthesis of amino |
| | | texture attributes. | acids, which are in increasing |
| | | Construction of genetic vectors | demand by the pharmaceutical |
| | | for controlled expression of RNA | industry. AraT plays a major role in |
| | | and/or protein, fusion protein | the conversion of aromatic amino |
| | | production, genetic modification, | acids to aroma compounds. AraT also |
| | | mutagenesis amplification of | has a major physiological role in the |
| | | genetic material or for other | biosynthesis of phenylalanine and |
| | | genetic or protein manipulations. | tyrosine. The enzymatic degradation |
| | | Altered survival characteristics: | of amino acids in cheese plays a |
| | | survival of industrial processes, | major role in cheese flavor |
| | | growth or storage in product | development. Indeed, degradation |
| | | formats, persistence in gut | products from aromatic, branched- |
| | | environment. | chain, and sulfurous amino acids |
| | | Altered metabolic properties. | have been identified in various |
| | | Altered probiotic attributes. | cheeses and highly contribute to their |
| | | Modified health properties | flavor or to off-flavors (Rijnen et al., |
| | | (including immunoregulatory, | Appl. Environ. Microbiol. 65: 4873-4880, |
| | | anticancer, gut health). | 1999). |
| | | Modified antibiotic resistance. | |
| | | Improved fermentation properties | |
| | | or other industrially useful | |
| | | processes. | |
| 26 | 108 | Altered cell wall or cell surface | Homologue of tagE, encoding |
| | | characteristics, structures or | poly(glycerol-phosphate) alpha- |
| | | functions. | glucosyltransferase (EC 2.4.1.52) |
| | | Modified adhesion to human or | also called uridine diphosphate- |
| | | animal cells or cell lines. | glucose poly-(glycerol phosphate) |
| | | Production of desirable flavors. | alpha-glucosyl transferase. TagE is |
| | | Modified flavor, aroma and/or | involved in techoic acid synthesis. |
| | | texture attributes. | Techoic acid is one component of the |
| | | Construction of genetic vectors | thick peptidoglycan layers in the cell |
| | | for controlled expression of RNA | wall of Gram-positive bacteria and is |
| | | and/or protein, fusion protein | susceptible to the enzyme lysozyme |
| | | production, genetic modification, | and to penicillin. |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved fermentation properties | |
| | | or other industrially useful | |
| | | processes. | |
| 27 | 109 | Altered amino acid metabolism. | Homologue to hisB, encoding a |
| | | Removal of undesirable flavor | histidine biosynthesis bifunctional |
| | | characteristics. | protein - includes: imidazoleglycerol- |
| | | Production of desirable flavors. | phosphate dehydratase (EC 4.2.1.19) |
| | | Modified flavor, aroma and/or | and histidinol-phosphatase (EC |
| | | texture attributes. | 3.1.3.15). HisB rearranges the |
| | | Construction of genetic vectors | imidazole glycerol phosphate by a |
| | | for controlled expression of RNA | redox-neutral dehydrative reaction to |
| | | and/or protein, fusion protein | imidazole acetol phosphate and |
| | | production, genetic modification, | catalyzes the dephosphorylation of |
| | | mutagenesis amplification of | hisidinol phosphate to histidinol, the |
| | | genetic material or for other | direct precursor of histidine. Because |
| | | genetic or protein manipulations. | hisB is absent from mammals, it has |
| | | Altered survival characteristics: | become a target for inhibition as part |
| | | survival of industrial processes, | of herbicide development (Barbosa et |
| | | growth or storage in product | al., Proc. Natl. Acad. Sci. USA |
| | | formats, persistence in gut | 99: 1859-1864, 2002). Amino acid |
| | | environment. | degradation products in various |
| | | Altered metabolic properties. | cheeses have been shown to greatly |
| | | Altered probiotic attributes. | contribute to flavor or to off-flavors |
| | | Modified health properties | (Rijnen et al., Appl. Environ. |
| | | (including immunoregulatory, | Microbiol. 65: 4873-4880, 1999). |
| | | anticancer, gut health). | |
| | | Modified antibiotic resistance. | |
| 28 | 110 | Altered amino acid metabolism. | Homologue of cysK, encoding |
| | | Removal of undesirable flavor | cysteine synthase (EC 4.2.99.8), also |
| | | characteristics. | known as O-acetyl-L-serine acetatelyase |
| | | Production of desirable flavors. | (EC 4.2.99.8). CysK catalyzes |
| | | Modified flavor, aroma and/or | the formation of L-cysteine, the last |
| | | texture attributes. | step of L-cysteine biosynthesis, from |
| | | Construction of genetic vectors | O-acetyl-L-serine and hydrogen |
| | | for controlled expression of RNA | sulfide. Cysteine synthase is involved |
| | | and/or protein, fusion protein | in the assimilatory sulfate reduction |
| | | production, genetic modification, | pathway and in the selenium |
| | | mutagenesis amplification of | incorporation into proteins, which |
| | | genetic material or for other | occurs mainly as selenocysteine, in |
| | | genetic or protein manipulations. | bacteria. Sulphur-containing amino |
| | | Altered survival characteristics: | acid metabolism is important for |
| | | survival of industrial processes, | development of aroma and flavor |
| | | growth or storage in product | compounds. |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 29 | 111 | Altered cell wall or cell surface | Homologue of enn protein. Enn has |
| | | characteristics, structures or | unique Ig-binding characteristics as it |
| | | functions. | reacts preferentially with human |
| | | Modified adhesion to human or | IgG3, the tlpC gene. Enn is a |
| | | animal cells or cell lines. | membrane protein with similarity to |
| | | Production of desirable flavors. | methyl-accepting chemotaxis proteins |
| | | Modified flavor, aroma and/or | and the streptococcal M proteins |
| | | texture attributes. | homologous with immunoglobulin- |
| | | Construction of genetic vectors | binding factors. The M proteins have |
| | | for controlled expression of RNA | been studied because of their |
| | | and/or protein, fusion protein | antiphagocytic function. |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| 30 | 112 | Altered amino acid metabolism. | Homologue of hisE, encoding a |
| | | Removal of undesirable flavor | histidine biosynthesis protein that |
| | | characteristics. | plays a role in histidine biosynthesis. |
| | | Production of desirable flavors. | Because hisE is absent in mammals, |
| | | Modified flavor, aroma and/or | it is a target for inhibition as part of |
| | | texture attributes. | herbicide development (Barbosa et |
| | | Construction of genetic vectors | al., Proc. Natl. Acad. Sci. USA |
| | | for controlled expression of RNA | 99: 1859-1864, 2002). Amino acid |
| | | and/or protein, fusion protein | degradation products in various |
| | | production, genetic modification, | cheeses have been shown to greatly |
| | | mutagenesis amplification of | contribute to flavor or to off-flavors |
| | | genetic material or for other | (Rijnen et al., Appl. Environ. |
| | | genetic or protein manipulations. | Microbiol. 65: 4873-4880, 1999). |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 30 | 113 | Altered amino acid metabolism. | Homologue of hisI, encoding a |
| | | Removal of undesirable flavor | histidine biosynthesis protein that |
| | | characteristics. | plays a role in histidine biosynthesis. |
| | | Production of desirable flavors. | Because hisI is absent in mammals, it |
| | | Modified flavor, aroma and/or | is a target for inhibition as part of |
| | | texture attributes. | herbicide development (Barbosa et |
| | | Construction of genetic vectors | al., Proc. Natl. Acad. Sci. USA |
| | | for controlled expression of RNA | 99: 1859-1864, 2002). Amino acid |
| | | and/or protein, fusion protein | degradation products in various |
| | | production, genetic modification, | cheeses have been shown to greatly |
| | | mutagenesis amplification of | contribute to flavor or to off-flavors |
| | | genetic material or for other | (Rijnen et al., Appl. Environ. |
| | | genetic or protein manipulations. | Microbiol. 65: 4873-4880, 1999). |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 31 | 114 | Production of desirable flavors. | Homologue of estA, encoding a |
| | | Modified flavor, aroma and/or | serine-dependent arylesterase (EC |
| | | texture attributes. | 3.1.1.2). EstA hydrolyzes a variety of |
| | | Construction of genetic vectors | ester compounds and prefers those |
| | | for controlled expression of RNA | with substituted phenyl alcohol or |
| | | and/or protein, fusion protein | short-chain fatty acid groups. |
| | | production, genetic modification, | Arylsesterases are responsible for the |
| | | mutagenesis amplification of | production of important flavor |
| | | genetic material or for other | compounds and intermediates. |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Modified lipid, glycolipid or free | |
| | | fatty acid levels or functional | |
| | | properties. | |
| | | Modified production of short | |
| | | chain fatty acids. | |
| | | Altered lipid metabolism. | |
| | | Altered probiotic attributes. | |
| | | Organisms or materials with | |
| | | improved health properties | |
| | | (including immunoregulatory, | |
| | | anticancer, gut health). | |
| 32 | 115 | Altered amino acid metabolism. | Homologue of glnA that encodes a |
| | | Removal of undesirable flavor | glutamine synthetase (EC 6.3.1.2), |
| | | characteristics. | also called glutamate-ammonia |
| | | Production of desirable flavors. | ligase. GlnA catalyzes the first step in |
| | | Modified flavor, aroma and/or | the conversion of inorganic nitrogen |
| | | texture attributes. | (ammonium) into its organic form |
| | | Construction of genetic vectors | glutamine (Gln). Bacterial glutamine |
| | | for controlled expression of RNA | synthetase export is associated with |
| | | and/or protein, fusion protein | pathogenicity and with the formation |
| | | production, genetic modification, | of a poly-L-glutamate/glutamine cell |
| | | mutagenesis amplification of | wall structure. Glutamine synthetase |
| | | genetic material or for other | is an enzyme that plays a central role |
| | | genetic or protein manipulations. | in the nitrogen metabolism. The |
| | | Altered survival characteristics: | enzyme and its products have roles in |
| | | survival of industrial processes, | flavor and growth. |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Altered cell wall or cell surface | |
| | | characteristics, structures or | |
| | | functions. | |
| 32 | 116 | Altered cell wall or cell surface | Homologue of Lipopolysaccharide |
| | | characteristics, structures or | synthesis protein yohJ. YohJ is |
| | | functions. | involved in techoic acid synthesis and |
| | | Modified adhesion to human or | important for cell wall functions |
| | | animal cells or cell lines. | including adhesion, immune cell |
| | | Production of desirable flavors. | interaction and product texture. |
| | | Modified flavor, aroma and/or | |
| | | texture attributes. | |
| | | Construction of genetic vectors | |
| | | for controlled expression of RNA | |
| | | and/or protein, fusion protein | |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| 33 | 117 | Production of desirable flavors. | Homologue of sorA. SorA encodes |
| | | Modified flavor, aroma and/or | the first protein of the |
| | | texture attributes. | phosphoenolpyruvate-dependent L- |
| | | Construction of genetic vectors | sorbose-specific phosphotransferase |
| | | for controlled expression of RNA | system (PTS). The ketose L-sorbose |
| | | and/or protein, fusion protein | is transported and phosphorylated |
| | | production, genetic modification, | through PTS. The enzyme is useful in |
| | | mutagenesis amplification of | carbohydrate-specific regulation of |
| | | genetic material or for other | gene expression and flavor |
| | | genetic or protein manipulations. | development. |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Modified carbohydrate levels or | |
| | | functional properties. | |
| | | Altered probiotic attributes. | |
| | | Organisms or materials with | |
| | | improved health properties | |
| | | (including immunoregulatory, | |
| | | anticancer, gut health). | |
| 33 | 118 | Production of desirable flavors. | Homologue of sorF, encoding D- |
| | | Modified flavor, aroma and/or | sorbitol-6-phosphate dehydrogenase, |
| | | texture attributes. | an enzyme of the L-sorbose |
| | | Construction of genetic vectors | metabolism. The ketose L-sorbose is |
| | | for controlled expression of RNA | transported and phosphorylated |
| | | and/or protein, fusion protein | through the phosphoenolpyruvate- |
| | | production, genetic modification, | dependent L-sorbose-specific |
| | | mutagenesis amplification of | phosphotransferase system (PTS). |
| | | genetic material or for other | Useful for carbohydrate-specific |
| | | genetic or protein manipulations. | regulation of gene expression (Yebra |
| | | Altered survival characteristics: | et al., J. Bacteriol. 182: 155-163, |
| | | survival of industrial processes, | 2000). |
| | | growth or storage in product | |
| | | formats, persistence in gut | |
| | | environment. | |
| | | Altered metabolic properties. | |
| | | Modified carbohydrate levels or | |
| | | functional properties. | |
| | | Altered probiotic attributes. | |
| | | Organisms or materials with | |
| | | improved health properties | |
| | | (including immunoregulatory, | |
| | | anticancer, gut health). | |
| 34 | 119 | Production of bioactive or | Homologue of pepA, encoding a |
| | | functional polypeptides. | glutamyl aminopeptidase, which |
| | | Removal of undesirable flavor | belongs to peptidase family M42 and |
| | | characteristics. | is required for optimal growth of |
| | | Production of desirable flavors. | Lactococcus lactis MG1363 in milk. |
| | | Modified flavor, aroma and/or | PepA has DNA-binding activity that |
| | | texture attributes. | functions in transcription control and |
| | | Construction of genetic vectors | plasmid dimer resolution. |
| | | for controlled expression of RNA | Aminopeptidases are involved in |
| | | and/or protein, fusion protein | many different functions in the cell, |
| | | production, genetic modification, | such as protein maturation, protein |
| | | mutagenesis amplification of | turnover, hydrolysis of regulatory |
| | | genetic material or for other | peptides, nitrogen nutrition, |
| | | genetic or protein manipulations. | modulation of gene expression etc. |
| | | Altered survival characteristics: | and, consequently, are considered |
| | | survival of industrial processes, | essential enzymes. The proteolytic |
| | | growth or storage in product | system of lactic acid bacteria is |
| | | formats, persistence in gut | essential for bacterial growth in milk |
| | | environment. | but also for the development of the |
| | | Altered metabolic properties or | organoleptic properties of dairy |
| | | regulation of metabolic | products. PepA is involved both in |
| | | pathways. | bacterial growth by supplying amino |
| | | Altered probiotic attributes. | acids, and in the development of |
| | | Organisms or materials with | flavor in dairy products, by |
| | | improved health properties | hydrolyzing peptides (including bitter |
| | | (including immunoregulatory, | peptides) and liberating aromatic |
| | | anticancer, gut health). | amino acids which are important |
| | | | precursors of aroma compounds |
| | | | (Fernandez-Espla and Rul, Eur. J. |
| | | | Biochem. 263: 502-510, 1999). |
| 35 | 120 | Altered amino acid metabolism. | Homologue of hom, encoding a |
| | | Removal of undesirable flavor | homoserine dehydrogenase (EC |
| | | characteristics. | 1.1.1.3), an enzyme of the threonine |
| | | Production of desirable flavors. | biosynthesis pathway. Threonine is |
| | | Modified flavor, aroma and/or | derived from aspartic acid. |
| | | texture attributes. | Conversion of aspartate to |
| | | Construction of genetic vectors | homoserine proceeds with ATP |
| | | for controlled expression of RNA | activation of the B-carboxyl group as |
| | | and/or protein, fusion protein | a mixed phosphoric anhydride |
| | | production, genetic modification, | followed by two sequential NADPH- |
| | | mutagenesis amplification of | dependent reductions to homoserine. |
| | | genetic material or for other | Phosphorylation of homoserine |
| | | genetic or protein manipulations. | provides the substrate |
| | | Altered survival characteristics: | phosphohomoserine which suffers a |
| | | survival of industrial processes, | stereospecific 1,2-transposition |
| | | growth or storage in product | reaction to give threonine. The |
| | | formats, persistence in gut | enzymatic degradation of amino acids |
| | | environment. | in cheese plays a major role in cheese |
| | | Altered metabolic properties. | flavor development. Amino acid |
| | | Altered probiotic attributes. | degradation products greatly |
| | | Modified health properties | contribute to flavor or to off-flavors |
| | | (including immunoregulatory, | (Rijnen et al., Appl. Environ. |
| | | anticancer, gut health). | Microbiol. 65: 4873-4880, 1999). |
| 35 | 121 | Altered cell wall or cell surface | Homologue of flotillin. Flotillins |
| | | characteristics, structures or | behave as resident integral membrane |
| | | functions. | protein components of caveolae |
| | | Modified adhesion to human or | which are plasmalemmal |
| | | animal cells or cell lines. | microdomains and are involved in |
| | | Production of desirable flavors. | vesicular trafficking and signal |
| | | Modified flavor, aroma and/or | transduction (Huang et al., Mol. |
| | | texture attributes. | Microbiol. 31: 361-371, 1999). |
| | | Construction of genetic vectors | Flotillins (also known as epidermal |
| | | for controlled expression of RNA | surface antigens (ESAs)) belong to |
| | | and/or protein, fusion protein | the family of caveolae-associated |
| | | production, genetic modification, | integral membrane proteins and may |
| | | mutagenesis amplification of | act as a scaffolding protein within |
| | | genetic material or for other | caveolar membranes. |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| 36 | 122 | Removal of undesirable flavor | Homologue of areB, encoding benzyl |
| | | characteristics. | alcohol dehydrogenase (EC 1.1.1.90). |
| | | Production of desirable flavors. | AreB catalyzes the oxidation of an |
| | | Modified flavor, aroma and/or | aromatic alcohol to an aromatic |
| | | texture attributes. | aldehyde. This enzyme enables |
| | | Construction of genetic vectors | bacteria to grow on a range of esters |
| | | for controlled expression of RNA | of aromatic alcohols and plays a role |
| | | and/or protein, fusion protein | in flavor development. |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Improved fermentation properties | |
| | | or other industrially useful | |
| | | processes. | |
| 37 | 123 | Altered amino acid metabolism. | Homologue of codB, encoding a |
| | | Removal of undesirable flavor | cytosine permease. CodB mediates |
| | | characteristics. | uptake of exogenously supplied |
| | | Production of desirable flavors. | cytosine. It belongs to the ABC |
| | | Modified flavor, aroma and/or | transporter family. The cytosine |
| | | texture attributes. | permease is an integral cytoplasmic |
| | | Construction of genetic vectors | membrane protein possessing several |
| | | for controlled expression of RNA | transmembrane-spanning domains. |
| | | and/or protein, fusion protein | The enzymatic degradation of amino |
| | | production, genetic modification, | acids in cheese plays a major role in |
| | | mutagenesis amplification of | cheese flavor development. Amino |
| | | genetic material or for other | acid degradation products greatly |
| | | genetic or protein manipulations. | contribute to flavor or to off-flavors |
| | | Altered survival characteristics: | (Rijnen et al., Appl. Environ. |
| | | survival of industrial processes, | Microbiol. 65: 4873-4880, 1999). |
| | | growth or storage in product | |
| | | formats, persistence in gut | |
| | | environment. | |
| | | Altered response to stress | |
| | | conditions. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| | | Improved fermentation properties | |
| | | or other industrially useful | |
| | | processes. | |
| 38 | 124 | Construction of genetic vectors | Homologue of hsp 18, encoding a |
| | | for controlled expression of RNA | low-molecular-weight protein |
| | | and/or protein, fusion protein | belonging into a family of small heat |
| | | production, genetic modification, | shock proteins. Hsp18 is induced not |
| | | mutagenesis amplification of | only by heat shock but also at the |
| | | genetic material or for other | onset of solventogenesis. Small heat |
| | | genetic or protein manipulations. | shock proteins (sHsps) are a diverse |
| | | Production of desirable flavors. | group of heat-induced proteins that |
| | | Modified flavor, aroma and/or | are conserved in prokaryotes and |
| | | texture attributes. | eukaryotes and are especially |
| | | Altered survival characteristics: | abundant in plants. Recent in vitro |
| | | survival of industrial processes, | data indicate that sHsps act as |
| | | growth or storage in product | molecular chaperones to prevent |
| | | formats, persistence in gut | thermal aggregation of proteins by |
| | | environment. | binding non-native intermediates, |
| | | Altered viability in response to | which can then be refolded in an |
| | | stress conditions. | ATP-dependent fashion by other |
| | | Altered metabolic properties or | chaperones (Lee and Vierling, Plant. |
| | | regulation of metabolic | Physiol. 122: 189-198, 2000). |
| | | pathways. | |
| | | Altered probiotic attributes. | |
| 39 | 125 | Altered cell wall or cell surface | Homologue to mccF, encoding a |
| | | characteristics, structures or | inner membrane associated protein of |
| | | functions. | bacteria. MccF determines resistance |
| | | Production of desirable flavors. | to exogenous microcin. Possible |
| | | Modified flavor, aroma and/or | action by preventing the reentering of |
| | | texture attributes. | the cell by exported translation |
| | | Construction of genetic vectors | inhibitor microcin C7 (Gonzalez- |
| | | for controlled expression of RNA | Pastor et al., J. Bacteriol. 177: 7131-7140, |
| | | and/or protein, fusion protein | 1995). |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| 40 | 126 | Production of desirable flavors. | Homologue of sorE, encoding an L- |
| | | Modified flavor, aroma and/or | sorbose-1-phosphate reductase. SorF |
| | | texture attributes. | is, together with D-glucitol-6- |
| | | Construction of genetic vectors | phosphate dehydrogenase, involved |
| | | for controlled expression of RNA | in the conversion of L-sorbose-1- |
| | | and/or protein, fusion protein | phosphate to D-fructose-6-phosphate. |
| | | production, genetic modification, | SorE is involved in flavor |
| | | mutagenesis amplification of | development and carbohydrate |
| | | genetic material or for other | metabolism. |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Modified carbohydrate levels or | |
| | | functional properties. | |
| | | Altered probiotic attributes. | |
| | | Organisms or materials with | |
| | | improved health properties | |
| | | (including immunoregulatory, | |
| | | anticancer, gut health). | |
| 41 | 127 | Production of desirable flavors. | Homologue of the iolB gene, |
| | | Modified flavor, aroma and/or | encoding a cis-acting catabolite- |
| | | texture attributes. | responsive element (cre) protein |
| | | Construction of genetic vectors | involved in the iol operon of the myo- |
| | | for controlled expression of RNA | inositol catabolism pathway. Myo- |
| | | and/or protein, fusion protein | inositol is abundant in nature, |
| | | production, genetic modification, | especially in soil. Various |
| | | mutagenesis amplification of | microorganisms are able to grow on |
| | | genetic material or for other | myo-inositol as the sole carbon |
| | | genetic or protein manipulations. | source. The expression of the iol |
| | | Altered survival characteristics: | operon is under glucose repression |
| | | survival of industrial processes, | (Miwa and Fujita, J. Bacteriol. |
| | | growth or storage in product | 183: 5877-5884, 2001). |
| | | formats, persistence in gut | |
| | | environment. | |
| | | Altered metabolic properties. | |
| | | Modified carbohydrate levels or | |
| | | functional properties. | |
| | | Altered cell wall or cell surface | |
| | | characteristics, structures or | |
| | | functions. | |
| | | Modified adhesion to human or | |
| | | animal cells or cell lines. | |
| | | Altered probiotic attributes. | |
| | | Organisms or materials with | |
| | | improved health properties | |
| | | (including immunoregulatory, | |
| | | anticancer, gut health). | |
| 41 | 128 | Production of desirable flavors. | Homologue of the iolC gene, |
| | | Modified flavor, aroma and/or | encoding 2-dehydro-3- |
| | | texture attributes. | deoxygluconokinase. IolC |
| | | Construction of genetic vectors | phosphorylates the 2-deoxy-5-keto- |
| | | for controlled expression of RNA | D-gluconic acid to 2-deoxy-5-keto-D- |
| | | and/or protein, fusion protein | gluconic acid 6-phosphate and is part |
| | | production, genetic modification, | of the iol operon of the myo-inositol |
| | | mutagenesis amplification of | catabolism pathway (Yoshida et al., |
| | | genetic material or for other | J. Bacteriol. 179: 4591-4598, 1997). |
| | | genetic or protein manipulations. | Myo-inositol is abundant in nature, |
| | | Altered survival characteristics: | especially in soil. Various |
| | | survival of industrial processes, | microorganisms are able to grow on |
| | | growth or storage in product | myo-inositol as the sole carbon |
| | | formats, persistence in gut | source. The expression of the iol |
| | | environment. | operon is under glucose repression |
| | | Altered metabolic properties. | (Miwa and Fujita, J. Bacteriol. |
| | | Modified carbohydrate levels or | 183: 5877-5884, 2001). |
| | | functional properties. | |
| | | Altered cell wall or cell surface | |
| | | characteristics, structures or | |
| | | functions. | |
| | | Modified adhesion to human or | |
| | | animal cells or cell lines. | |
| | | Altered probiotic attributes. | |
| | | Organisms or materials with | |
| | | improved health properties | |
| | | (including immunoregulatory, | |
| | | anticancer, gut health). | |
| 41 | 129 | Production of desirable flavors. | Homologue of the iolE gene, |
| | | Modified flavor, aroma and/or | encoding a protein involved in the iol |
| | | texture attributes. | operon of the myo-inositol |
| | | Construction of genetic vectors | catabolism pathway. Myo-inositol is |
| | | for controlled expression of RNA | abundant in nature, especially in soil. |
| | | and/or protein, fusion protein | Various microorganisms are able to |
| | | production, genetic modification, | grow on myo-inositol as the sole |
| | | mutagenesis amplification of | carbon source. The expression of the |
| | | genetic material or for other | iol operon is under glucose repression |
| | | genetic or protein manipulations. | (Miwa and Fujita, J. Bacteriol. |
| | | Altered survival characteristics: | 183: 5877-5884, 2001). |
| | | survival of industrial processes, | |
| | | growth or storage in product | |
| | | formats, persistence in gut | |
| | | environment. | |
| | | Altered metabolic properties. | |
| | | Modified carbohydrate levels or | |
| | | functional properties. | |
| | | Altered cell wall or cell surface | |
| | | characteristics, structures or | |
| | | functions. | |
| | | Modified adhesion to human or | |
| | | animal cells or cell lines. | |
| | | Altered probiotic attributes. | |
| | | Organisms or materials with | |
| | | improved health properties | |
| | | (including immunoregulatory, | |
| | | anticancer, gut health). | |
| 41 | 130 | Production of desirable flavors. | Homologue of the iolG gene, |
| | | Modified flavor, aroma and/or | encoding inositol dehydrogenase (EC |
| | | texture attributes. | 1.1.1.18). IolG catalyzes the first |
| | | Construction of genetic vectors | reaction of the inositol catabolism, |
| | | for controlled expression of RNA | the dehydrogenation of myo-inositol |
| | | and/or protein, fusion protein | into 2-keto-myo-inositol (2-inosose) |
| | | production, genetic modification, | (Yoshida et al., J. Bacteriol. |
| | | mutagenesis amplification of | 179: 4591-4598, 1997). IolG is part of |
| | | genetic material or for other | the iol operon of the myo-inositol |
| | | genetic or protein manipulations. | catabolism pathway. Myo-inositol is |
| | | Altered survival characteristics: | abundant in nature, especially in soil. |
| | | survival of industrial processes, | Various microorganisms are able to |
| | | growth or storage in product | grow on myo-inositol as the sole |
| | | formats, persistence in gut | carbon source. The expression of the |
| | | environment. | iol operon is under glucose repression |
| | | Altered metabolic properties. | (Miwa and Fujita, J. Bacteriol. |
| | | Modified carbohydrate levels or | 183: 5877-5884, 2001). |
| | | functional properties. | |
| | | Altered cell wall or cell surface | |
| | | characteristics, structures or | |
| | | functions. | |
| | | Modified adhesion to human or | |
| | | animal cells or cell lines. | |
| | | Altered probiotic attributes. | |
| | | Organisms or materials with | |
| | | improved health properties | |
| | | (including immunoregulatory, | |
| | | anticancer, gut health). | |
| 41 | 131 | Production of desirable flavors. | Homologue of the iolJ gene, |
| | | Modified flavor, aroma and/or | encoding fructose-bisphosphate |
| | | texture attributes. | aldolase (EC 4.1.2.13) or tagatose- |
| | | Construction of genetic vectors | bisphosphate aldolase (4.1.2.40). IolJ |
| | | for controlled expression of RNA | cleaves 2-deoxy-5-keto-D-gluconic |
| | | and/or protein, fusion protein | acid 6-phosphate to yield |
| | | production, genetic modification, | dihydroxyacetone phosphate and |
| | | mutagenesis amplification of | malonic semialdehyde and is part of |
| | | genetic material or for other | the iol operon of the myo-inositol |
| | | genetic or protein manipulations. | catabolism pathway (Yoshida et al., |
| | | Altered survival characteristics: | J. Bacteriol. 179: 4591-4598, 1997). |
| | | survival of industrial processes, | Myo-inositol is abundant in nature, |
| | | growth or storage in product | especially in soil. Various |
| | | formats, persistence in gut | microorganisms are able to grow on |
| | | environment. | myo-inositol as the sole carbon |
| | | Altered metabolic properties. | source. The expression of the iol |
| | | Modified carbohydrate levels or | operon is under glucose repression |
| | | functional properties. | (Miwa and Fujita, J. Bacteriol. |
| | | Altered cell wall or cell surface | 183: 5877-5884, 2001). |
| | | characteristics, structures or | |
| | | functions. | |
| | | Modified adhesion to human or | |
| | | animal cells or cell lines. | |
| | | Altered probiotic attributes. | |
| | | Organisms or materials with | |
| | | improved health properties | |
| | | (including immunoregulatory, | |
| | | anticancer, gut health). | |
| 41 | 132 | Altered amino acid metabolism. | Homologue of mmsA or iolA, |
| | | Removal of undesirable flavor | encoding methylmalonate- |
| | | characteristics. | semialdehyde dehydrogenase. IolA |
| | | Production of desirable flavors. | converts malonic semialdehyde into |
| | | Modified flavor, aroma and/or | acetyl CoA and CO₂, the final step of |
| | | texture attributes. | inositol degradation (Yoshida et al., |
| | | Construction of genetic vectors | J. Bacteriol. 179: 4591-4598, 1997). |
| | | for controlled expression of RNA | Myo-Inositol is abundant in nature, |
| | | and/or protein, fusion protein | especially in soil. Various |
| | | production, genetic modification, | microorganisms are able to grow on |
| | | mutagenesis amplification of | myo-inositol as the sole carbon |
| | | genetic material or for other | source. The expression of the iol |
| | | genetic or protein manipulations. | operon is under glucose repression |
| | | Altered survival characteristics: | (Miwa and Fujita, J. Bacteriol. |
| | | survival of industrial processes, | 183: 5877-5884, 2001). The enzyme |
| | | growth or storage in product | is also required for growth on valine |
| | | formats, persistence in gut | and isoleucine as it is an acylating |
| | | environment. | enzyme that converts both propanal |
| | | Altered metabolic properties. | and 2-Methyl-3-oxopropanoate to |
| | | Modified carbohydrate levels or | Propanoyl-CoA. The enzymatic |
| | | functional properties. | degradation of amino acids in cheese |
| | | Altered cell wall or cell surface | plays a major role in cheese flavor |
| | | characteristics, structures or | development. Amino acid |
| | | functions. | degradation products greatly |
| | | Modified adhesion to human or | contribute to flavor or to off-flavors |
| | | animal cells or cell lines. | (Rijnen et al., Appl. Environ. |
| | | Altered probiotic attributes. | Microbiol. 65: 4873-4880, 1999). |
| | | Organisms or materials with | |
| | | improved health properties | |
| | | (including immunoregulatory, | |
| | | anticancer, gut health). | |
| 42 | 133 | Altered amino acid metabolism. | Homologue of hisF, encoding |
| | | Removal of undesirable flavor | imidazole glycerol phosphate |
| | | characteristics. | synthase subunit hisF (EC 4.1.3.—) |
| | | Production of desirable flavors. | also called IGP synthase cyclase |
| | | Modified flavor, aroma and/or | subunit. HisF links histidine and de |
| | | texture attributes. | novo purine biosynthesis and is a |
| | | Construction of genetic vectors | member of the glutamine |
| | | for controlled expression of RNA | amidotransferase family. |
| | | and/or protein, fusion protein | The enzymatic degradation of amino |
| | | production, genetic modification, | acids in cheese plays a major role in |
| | | mutagenesis amplification of | cheese flavor development. Amino |
| | | genetic material or for other | acid degradation products greatly |
| | | genetic or protein manipulations. | contribute to flavor or to off-flavors |
| | | Altered survival characteristics: | (Rijnen et al., Appl. Environ. |
| | | survival of industrial processes, | Microbiol. 65: 4873-4880, 1999). |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 43 | 134 | Construction of genetic vectors | Homologue of yqcM, encoding |
| | | for controlled expression of RNA | arsenate reductase (EC 1.97.1.5). |
| | | and/or protein, fusion protein | YqcM reduces the arsenate ion |
| | | production, genetic modification, | (H₂AsO) to arsenite ion (AsO). |
| | | mutagenesis amplification of | Arsenate is an abundant oxyanion |
| | | genetic material or for other | that, because of its ability to mimic |
| | | genetic or protein manipulations. | the phosphate group, is toxic to cells. |
| | | Production of desirable flavors. | Arsenate reductase participates to |
| | | Modified flavor, aroma and/or | achieve arsenate resistance in both |
| | | texture attributes. | prokaryotes and yeast by reducing |
| | | Altered survival characteristics: | arsenate to arsenite; the arsenite is |
| | | survival of industrial processes, | then exported by a specific |
| | | growth or storage in product | transporter. Arsenite reductase is |
| | | formats, persistence in gut | coupled to the glutathione and |
| | | environment. | glutaredoxin system for its enzyme |
| | | Altered viability in response to | activity (Bennett et al., Proc. Natl. |
| | | stress conditions. | Acad. Sci. USA 98: 13577-13582, |
| | | Altered metabolic properties or | 2001). |
| | | regulation of metabolic | |
| | | pathways. | |
| | | Altered probiotic attributes. | |
| | | Improved fermentation properties | |
| | | or other industrially useful | |
| | | processes. | |
| | | Modified health properties | |
| | | (including immunoregulatory, | |
| | | anticancer, gut health). | |
| 44 | 135 | Altered amino acid metabolism. | Homologue of cna, encoding a |
| | | Removal of undesirable flavor | collagen adhesin. Cna mediates |
| | | characteristics. | attachment of bacterial cells to |
| | | Production of desirable flavors. | collagen-containing substrata and is |
| | | Modified flavor, aroma and/or | attached to the cell wall |
| | | texture attributes. | peptidoglycan by an amide bond. |
| | | Construction of genetic vectors | |
| | | for controlled expression of RNA | |
| | | and/or protein, fusion protein | |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| 45, 65 | 136, 159 | Production of bioactive or | Homologue of pepQ, encoding a |
| | | functional polypeptides. | Xaa-Pro dipeptidase (EC 3.4.13.9). |
| | | Removal of undesirable flavor | PepQ hydrolyzes Xaa-Pro dipeptides |
| | | characteristics. | (but not Pro-Pro) and also acts on |
| | | Production of desirable flavors. | aminoacyl-hydroxyproline analogs |
| | | Modified flavor, aroma and/or | This peptidase belongs to peptidase |
| | | texture attributes. | family M24A (methionyl |
| | | Construction of genetic vectors | aminopeptidase family). It has a |
| | | for controlled expression of RNA | potential use in the dairy industry as a |
| | | and/or protein, fusion protein | cheese-ripening agent since proline |
| | | production, genetic modification, | release from proline-containing |
| | | mutagenesis amplification of | peptides in cheese reduces bitterness. |
| | | genetic material or for other | The proteolytic system of lactic acid |
| | | genetic or protein manipulations. | bacteria is essential for bacterial |
| | | Altered survival characteristics: | growth in milk but also for the |
| | | survival of industrial processes, | development of the organoleptic |
| | | growth or storage in product | properties of dairy products. PepQ is |
| | | formats, persistence in gut | involved both in bacterial growth by |
| | | environment. | supplying amino acids, and in the |
| | | Altered metabolic properties or | development of flavor in dairy |
| | | regulation of metabolic | products, by hydrolyzing peptides |
| | | pathways. | (including bitter peptides) and |
| | | Altered probiotic attributes. | liberating aromatic amino acids |
| | | Organisms or materials with | which are important precursors of |
| | | improved health properties | aroma compounds (Fernandez-Espla |
| | | (including immunoregulatory) | and Rul, Eur. J. Biochem. 263: 502-510, |
| | | | 1999). |
| 46 | 137 | Altered amino acid metabolism. | Homologue of argG, encoding a |
| | | Removal of undesirable flavor | argininosuccinate synthase (EC |
| | | characteristics. | 6.3.4.5). ArgG catalyzes the |
| | | Production of desirable flavors. | penultimate step of the arginine |
| | | Modified flavor, aroma and/or | biosynthesis. It belongs to the |
| | | texture attributes. | argininosuccinate synthase family. |
| | | Construction of genetic vectors | The enzymatic degradation of amino |
| | | for controlled expression of RNA | acids in cheese plays a major role in |
| | | and/or protein, fusion protein | cheese flavor development. Amino |
| | | production, genetic modification, | acid degradation products greatly |
| | | mutagenesis amplification of | contribute to flavor or to off-flavors |
| | | genetic material or for other | (Rijnen et al., Appl. Environ. |
| | | genetic or protein manipulations. | Microbiol. 65: 4873-4880, 1999). |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 46 | 138 | Altered amino acid metabolism. | Homologue of argH, encoding |
| | | Removal of undesirable flavor | argininosuccinate lyase (EC 4.3.2.1). |
| | | characteristics. | ArgH catalyzes the last step in the |
| | | Production of desirable flavors. | arginine biosynthesis. |
| | | Modified flavor, aroma and/or | Argininosuccinate lyase also |
| | | texture attributes. | participates in the urea cycle, the |
| | | Construction of genetic vectors | major pathway for the detoxification |
| | | for controlled expression of RNA | of ammonia, where it catalyzes the |
| | | and/or protein, fusion protein | reversible breakdown of |
| | | production, genetic modification, | argininosuccinic acid into arginine |
| | | mutagenesis amplification of | and fumarate. The enzymatic |
| | | genetic material or for other | degradation of amino acids in cheese |
| | | genetic or protein manipulations. | plays a major role in cheese flavor |
| | | Altered survival characteristics: | development. Amino acid |
| | | survival of industrial processes, | degradation products greatly |
| | | growth or storage in product | contribute to flavor or to off-flavors |
| | | formats, persistence in gut | (Rijnen et al., Appl. Environ. |
| | | environment. | Microbiol. 65: 4873-4880, 1999). |
| | | Altered metabolic properties. | |
| | | Altered probiotic attributes. | |
| | | modified health properties | |
| | | (including immunoregulatory, | |
| | | anticancer, gut health). | |
| | | Modified antibiotic resistance. | |
| | | Improved fermentation properties | |
| | | or other industrially useful | |
| | | processes. | |
| 47 | 139 | Construction of genetic vectors | Homologue of purL, encoding a |
| | | for controlled expression of RNA | phosphoribosylformylglycinamidine |
| | | and/or protein, fusion protein | (FGAM) synthetase (EC 6.3.5.3). |
| | | production, genetic modification, | PurL catalyzes the fourth step in the |
| | | mutagenesis amplification of | biosynthesis of purines. It is involved |
| | | genetic material or for other | in multistress resistance. Purines play |
| | | genetic or protein manipulations. | essential roles in many cellular |
| | | Production of desirable flavors. | functions, including DNA replication, |
| | | Modified flavor, aroma and/or | transcription, intra- and extra-cellular |
| | | texture attributes. | signaling, energy metabolism, and as |
| | | Altered survival characteristics: | coenzymes for many biochemical |
| | | survival of industrial processes, | reactions. |
| | | growth or storage in product | |
| | | formats, persistence in gut | |
| | | environment. | |
| | | Altered viability in response to | |
| | | stress conditions. | |
| | | Altered metabolic properties or | |
| | | regulation of metabolic | |
| | | pathways. | |
| | | Altered probiotic attributes. | |
| 48 | 140 | Altered amino acid metabolism. | Homologue of hisH, encoding a |
| | | Removal of undesirable flavor | imidazole glycerol phosphate |
| | | characteristics. | synthase subunit that is also known as |
| | | Production of desirable flavors. | IGP synthase glutamine |
| | | Modified flavor, aroma and/or | amidotransferase subunit. HisH |
| | | texture attributes. | catalyzes the fifth step of the histidine |
| | | Construction of genetic vectors | biosynthesis. The hisH subunit |
| | | for controlled expression of RNA | provides the glutamine |
| | | and/or protein, fusion protein | amidotransferase activity that |
| | | production, genetic modification, | produces the ammonia necessary to |
| | | mutagenesis amplification of | hisF for the synthesis of IGP and |
| | | genetic material or for other | AICAR. The enzymatic degradation |
| | | genetic or protein manipulations. | of amino acids in cheese plays a |
| | | Altered survival characteristics: | major role in cheese flavor |
| | | survival of industrial processes, | development. Amino acid |
| | | growth or storage in product | degradation products greatly |
| | | formats, persistence in gut | contribute to flavor or to off-flavors |
| | | environment. | (Rijnen et al., Appl. Environ. |
| | | Altered metabolic properties. | Microbiol. 65: 4873-4880, 1999). |
| | | Altered probiotic attributes. | |
| | | Modified health properties | |
| | | (including immunoregulatory, | |
| | | anticancer, gut health). | |
| | | Modified antibiotic resistance. | |
| 49 | 141 | Construction of genetic vectors | Homologue of ndK, encoding |
| | | for controlled expression of RNA | nucleoside-diphosphate kinase (EC |
| | | and/or protein, fusion protein | 2.7.4.6), which converts |
| | | production, genetic modification, | (deoxy)ribonucleoside diphosphates |
| | | mutagenesis amplification of | into their corresponding |
| | | genetic material or for other | triphosphates. NdK is an ubiquitous |
| | | genetic or protein manipulations. | and nonspecific enzyme but is an |
| | | Production of desirable flavors. | important cellular enzyme that |
| | | Modified flavor, aroma and/or | monitors and maintains nucleotide |
| | | texture attributes. | pools and has been implicated in a |
| | | Altered survival characteristics: | number of regulatory processes, |
| | | survival of industrial processes, | including signal transduction, |
| | | growth or storage in product | development and cell surface |
| | | formats, persistence in gut | polysaccharide synthesis. |
| | | environment. | |
| | | Altered viability in response to | |
| | | stress conditions. | |
| | | Altered metabolic properties or | |
| | | regulation of metabolic | |
| | | pathways. | |
| | | Altered probiotic attributes. | |
| 50 | 142 | Altered cell wall or cell surface | Homologue of PrtB, a PII-type |
| | | characteristics, structures or | proteinase precursor (Lactocepin) |
| | | functions. | also called cell wall-associated serine |
| | | Modified adhesion to human or | proteinase (EC 3.4.21.96). PrtB |
| | | animal cells or cell lines. | breaks down milk proteins during the |
| | | Production of desirable flavors. | growth of the bacteria on milk and |
| | | Modified flavor, aroma and/or | that provides the peptides essential |
| | | texture attributes. | for cell growth. It has endopeptidase |
| | | Construction of genetic vectors | activity with very broad specificity. It |
| | | for controlled expression of RNA | is best known for its action on |
| | | and/or protein, fusion protein | caseins, although it has been shown |
| | | production, genetic modification, | to hydrolyze hemoglobin and |
| | | mutagenesis amplification of | oxidized insulin b-chain. Lactocepin |
| | | genetic material or for other | is a type I membrane protein, located |
| | | genetic or protein manipulations. | in the cell wall and belongs to |
| | | Altered survival characteristics: | peptidase family S8; also known as |
| | | survival of industrial processes, | the Subtilase Family. Lactocepin is |
| | | growth or storage in product | responsible for the hydrolysis of |
| | | formats, persistence in gut | casein in milk and specificity |
| | | environment. | differences between lactocepins from |
| | | Altered metabolic properties. | different starter strains may be partly |
| | | Altered probiotic attributes. | responsible for imparting different |
| | | Modified health properties | flavor qualities to cheese (Broadbent |
| | | (including immunoregulatory, | et al., Appl. Environ. Microbiol. |
| | | anticancer, gut health). | 68: 1778-1785, 2002). |
| | | Modified antibiotic resistance. | |
| | | Improved fermentation properties | |
| | | or other industrially useful | |
| | | processes. | |
| 51 | 143 | Altered amino acid metabolism. | Homologue of aspB, encoding an |
| | | Removal of undesirable flavor | aspartate aminotransferase (EC: |
| | | characteristics. | 2.6.1.1), also called aspartate |
| | | Production of desirable flavors. | transaminase. AspB catalyzes the |
| | | Modified flavor, aroma and/or | amino group transfer between amino |
| | | texture attributes. | acids and 2-oxo acids and that plays a |
| | | Construction of genetic vectors | central role in amino acid metabolism |
| | | for controlled expression of RNA | in organisms. The transferase is |
| | | and/or protein; fusion protein | important for the metabolism of |
| | | production, genetic modification, | amino acids and Krebs cycle related |
| | | mutagenesis amplification of | organic acids. It plays a role in the |
| | | genetic material or for other | production of important flavor |
| | | genetic or protein manipulations. | determinants. The enzymatic |
| | | Altered survival characteristics: | degradation of amino acids in cheese |
| | | survival of industrial processes, | plays a major role in cheese flavor |
| | | growth or storage in product | development. Amino acid |
| | | formats, persistence in gut | degradation products greatly |
| | | environment. | contribute to flavor or to off-flavors |
| | | Altered metabolic properties. | (Rijnen et al., Appl. Environ. |
| | | Altered probiotic attributes. | Microbiol. 65: 4873-4880, 1999). |
| | | Modified health properties | |
| | | (including immunoregulatory, | |
| | | anticancer, gut health). | |
| | | Modified antibiotic resistance. | |
| | | Improved fermentation properties | |
| | | or other industrially useful | |
| | | processes. | |
| 52 | 144 | Production of desirable flavors. | Homologue of iolF, encoding an |
| | | Modified flavor, aroma and/or | inositol transporter (TC#: |
| | | texture attributes. | 2.A.1.1.27). IolF transports myo- |
| | | Construction of genetic vectors | inositol into the bacterial cell. IolF is |
| | | for controlled expression of RNA | part of the iol operon of the myo- |
| | | and/or protein, fusion protein | inositol catabolism pathway. Myo- |
| | | production, genetic modification, | inositol is abundant in nature, |
| | | mutagenesis amplification of | especially in soil. Various |
| | | genetic material or for other | microorganisms are able to grow on |
| | | genetic or protein manipulations. | myo-inositol as the sole carbon |
| | | Altered survival characteristics: | source. The expression of the iol |
| | | survival of industrial processes, | operon is under glucose repression |
| | | growth or storage in product | (Miwa and Fujita, J. Bacteriol. |
| | | formats, persistence in gut | 183: 5877-5884, 2001). |
| | | environment. | |
| | | Altered metabolic properties. | |
| | | Modified carbohydrate levels or | |
| | | fnctional properties. | |
| | | Altered cell wall or cell surface | |
| | | characteristics, structures or | |
| | | functions. | |
| | | Modified adhesion to human or | |
| | | animal cells or cell lines. | |
| | | Altered probiotic attributes. | |
| | | Organisms or materials with | |
| | | improved health properties | |
| | | (including immunoregulatory, | |
| | | anticancer, gut health). | |
| 54 | 146 | Altered cell wall or cell surface | Homologue of mga4, a positive |
| | | characteristics, structures or | regulatory protein that acts as a |
| | | functions. | component of a signal transducing |
| | | Modified adhesion to human or | system. Positive regulatory proteins |
| | | animal cells or cell lines. | or activator proteins bind in their |
| | | Production of desirable flavors. | active state to DNA in the promoter |
| | | Modified flavor, aroma and/or | region and help RNA polymerase to |
| | | texture attributes. | bind and transcribe that gene. Mga4 |
| | | Construction of genetic vectors | initiates transcription of surface- |
| | | for controlled expression of RNA | associated/virulence factors. |
| | | and/or protein, fusion protein | |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| | | Regulation of polysaccharide | |
| | | production, adhesion, immune | |
| | | modulation. | |
| 55 | 147 | Altered amino acid metabolism. | Homologue of BH3554, encoding a |
| | | Removal of undesirable flavor | carboxylesterase (3.1.1.1). BH3554 |
| | | characteristics. | hydrolyzes carboxylic ester bonds |
| | | Production of desirable flavors. | with relatively broad substrate |
| | | Modified flavor, aroma and/or | specificity. It is involved in amino |
| | | texture attributes. | acid metabolism and flavor. The |
| | | Construction of genetic vectors | enzymatic degradation of amino acids |
| | | for controlled expression of RNA | in cheese plays a major role in cheese |
| | | and/or protein, fusion protein | flavor development. Amino acid |
| | | production, genetic modification, | degradation products greatly |
| | | mutagenesis amplification of | contribute to flavor or to off-flavors |
| | | genetic material or for other | (Rijnen et al., Appl. Environ. |
| | | genetic or protein manipulations. | Microbiol. 65: 4873-4880, 1999). |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved fermentation properties | |
| | | or other industrially useful | |
| | | processes. | |
| 56 | 148 | Altered amino acid metabolism. | Homologue of Amd1, encoding an |
| | | Removal of undesirable flavor | aminoacylase (EC 3.5.1.14). Amd1 |
| | | characteristics. | deacetylates acylated amino acids. It |
| | | Production of desirable flavors. | plays a role in the production of |
| | | Modified flavor, aroma and/or | important flavor determinants. The |
| | | texture attributes. | enzymatic degradation of amino acids |
| | | Construction of genetic vectors | in cheese plays a major role in cheese |
| | | for controlled expression of RNA | flavor development. Amino acid |
| | | and/or protein, fusion protein | degradation products greatly |
| | | production, genetic modification, | contribute to flavor or to off-flavors |
| | | mutagenesis amplification of | (Rijnen et al., Appl. Environ. |
| | | genetic material or for other | Microbiol. 65: 4873-4880, 1999). |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved fermentation properties | |
| | | or other industrially useful | |
| | | processes. | |
| 57 | 149 | Altered cell wall or cell surface | Homologue of tmpA, encoding a |
| | | characteristics, structures or | putative transmembrane protein. |
| | | functions. | TmpA plays a role in adhesion and is |
| | | Modified adhesion to human or | part of an operon containing the |
| | | animal cells or cell lines. | mapA gene. |
| | | Production of desirable flavors. | |
| | | Modified flavor, aroma and/or | |
| | | texture attributes. | |
| | | Construction of genetic vectors | |
| | | for controlled expression of RNA | |
| | | and/or protein, fusion protein | |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | including immunoregulatory, | |
| | | anticancer, gut health). | |
| | | Modified antibiotic resistance. | |
| | | Improved fermentation properties | |
| | | or other industrially useful | |
| | | processes. | |
| 58 | 150 | Construction of genetic vectors | Homologue of npr, encoding a |
| | | for controlled expression of RNA | NADH peroxidase (EC 1.11.1.1). Npr |
| | | and/or protein, fusion protein | utilizes hydrogen peroxide to create |
| | | production, genetic modification, | water and nicotinamide adenine |
| | | mutagenesis amplification of | dinucleotide (NADH) from its |
| | | genetic material or for other | oxidized form (NAD). Metabolism of |
| | | genetic or protein manipulations. | co-factors such as NADH can greatly |
| | | Production of desirable flavors. | influence the speed and type of |
| | | Modified flavor, aroma and/or | metabolic pathway utilized under |
| | | texture attributes. | different redox conditions, and can |
| | | Altered survival characteristics: | therefore influence flavor and/or |
| | | survival of industrial processes, | functionality. |
| | | growth or storage in product | |
| | | formats, persistence in gut | |
| | | environment. | |
| | | Altered viability in response to | |
| | | stress conditions. | |
| | | Altered metabolic properties or | |
| | | regulation of metabolic | |
| | | pathways. | |
| | | Altered probiotic attributes. | |
| | | Improved fermentation properties | |
| | | or other industrially useful | |
| | | processes. | |
| 59, 60 | 151, 152 | Altered amino acid metabolism. | Homologue of nifS. NifS is involved |
| | | Removal of undesirable flavor | in cysteine metabolism and |
| | | characteristics. | development of flavor compounds. |
| | | Production of desirable flavors. | The enzymatic degradation of amino |
| | | Modified flavor, aroma and/or | acids in cheese plays a major role in |
| | | texture attributes. | cheese flavor development. Amino |
| | | Construction of genetic vectors | acid degradation products greatly |
| | | for controlled expression of RNA | contribute to flavor or to off-flavors |
| | | and/or protein, fusion protein | (Rijnen et al., Appl. Environ. |
| | | production, genetic modification, | Microbiol. 65: 4873-4880, 1999). |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved fermentation properties | |
| | | or other industrially useful | |
| | | processes. | |
| 61 | 153 | Production of desirable flavors. | Homologue of fabA, encoding a 3- |
| | | Modified flavor, aroma and/or | hydroxydecanoyl-ACP dehydratase. |
| | | texture attributes. | FabA introduces cis unsaturation into |
| | | Construction of genetic vectors | fatty acids during saturated fatty acid |
| | | for controlled expression of RNA | biosynthesis. The dehydratase |
| | | and/or protein, fusion protein | belongs to the thioester dehydratase |
| | | production, genetic modification, | family. Free fatty acids are important |
| | | mutagenesis amplification of | in providing flavor-bearing |
| | | genetic material or for other | compounds for dairy products such as |
| | | genetic or protein manipulations. | cheese, and have a significant role in |
| | | Altered survival characteristics: | both flavor and texture. Used |
| | | survival of industrial processes, | extensively in wide range of |
| | | growth or storage in product | convenience foods. Short chain fatty |
| | | formats, persistence in gut | acids are known to have a variety of |
| | | environment. | health impacts. |
| | | Altered metabolic properties. | |
| | | Modified lipid, glycolipid or free | |
| | | fatty acid levels or functional | |
| | | properties. | |
| | | Modified production of short | |
| | | chain fatty acids. | |
| | | Altered lipid metabolism. | |
| | | Altered probiotic attributes. | |
| | | Organisms or materials with | |
| | | improved health properties | |
| | | (including immunoregulatory, | |
| | | anticancer, gut health) | |
| 62 | 154 | Altered cell wall or cell surface | Homologue of aggH, encoding an |
| | | characteristics, structures or | autoaggregation mediating protein. |
| | | functions. | AggH contains a region of similarity |
| | | Modified adhesion to human or | to ATP-dependent DEAD-box |
| | | animal cells or cell lines. | helicase. The protein is involved in |
| | | Production of desirable flavors. | genetic exchange, pathogen exclusion |
| | | Modified flavor, aroma and/or | and persistence in the gut |
| | | texture attributes. | environment by promoting |
| | | Construction of genetic vectors | aggregation between bacteria. |
| | | for controlled expression of RNA | |
| | | and/or protein, fusion protein | |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| | | Improved fermentation properties | |
| | | or other industrially useful | |
| | | processes. | |
| 63 | 155 | Production of desirable flavors. | Homologue of the iolH gene, |
| | | Modified flavor, aroma and/or | encoding a protein involved in the iol |
| | | texture attributes. | operon of the myo-inositol |
| | | Construction of genetic vectors | catabolism pathway. Myo--inositol is |
| | | for controlled expression of RNA | abundant in nature, especially in soil. |
| | | and/or protein, fusion protein | Various microorganisms are able to |
| | | production, genetic modification, | grow on myo-inositol as the sole |
| | | mutagenesis amplification of | carbon source. The expression of the |
| | | genetic material or for other | iol operon is under glucose repression |
| | | genetic or protein manipulations. | (Miwa and Fujita, J. Bacteriol. |
| | | Altered survival characteristics: | 183: 5877-5884, 2001) |
| | | survival of industrial processes, | |
| | | growth or storage in product | |
| | | formats, persistence in gut | |
| | | environment. | |
| | | Altered metabolic properties. | |
| | | Modified carbohydrate levels or | |
| | | functional properties. | |
| | | Altered cell wall or cell surface | |
| | | characteristics, structures or | |
| | | functions. | |
| | | Modified adhesion to human or | |
| | | animal cells or cell lines. | |
| | | Altered probiotic attributes. | |
| | | Organisms or materials with | |
| | | improved health properties | |
| | | (including immunoregulatory, | |
| | | anticancer, gut health). | |
| 63 | 156 | Production of desirable flavors. | Homologue of the iolL gene, |
| | | Modified flavor, aroma and/or | encoding a protein involved in the iol |
| | | texture attributes. | operon of the myo-inositol |
| | | Construction of genetic vectors | catabolism pathway. Myo--nositol is |
| | | for controlled expression of RNA | abundant in nature, especially in soil. |
| | | and/or protein, fusion protein | Various microorganisms are able to |
| | | production, genetic modification, | grow on myo-inositol as the sole |
| | | mutagenesis amplification of | carbon source. The expression of the |
| | | genetic material or for other | iol operon is under glucose repression |
| | | genetic or protein manipulations. | (Miwa and Fujita, J. Bacteriol. |
| | | Altered survival characteristics: | 183: 5877-5884, 2001) |
| | | survival of industrial processes, | |
| | | growth or storage in product | |
| | | formats, persistence in gut | |
| | | environment. | |
| | | Altered metabolic properties. | |
| | | Modified carbohydrate levels or | |
| | | functional properties. | |
| | | Altered cell wall or cell surface | |
| | | characteristics, structures or | |
| | | functions. | |
| | | Modified adhesion to human or | |
| | | animal cells or cell lines. | |
| | | Altered probiotic attributes. | |
| | | Organisms or materials with | |
| | | improved health properties | |
| | | (including immunoregulatory, | |
| | | anticancer, gut health). | |
| 64 | 157 | Removal of undesirable flavor | Homologue of citX, encoding apo- |
| | | characteristics. | citrate lyase phosphoribosyl- |
| | | Modified flavor, aroma, texture | dephospho-CoA transferase (Apo- |
| | | attributes. | ACP nucleodityltransferase; EC |
| | | Construction of genetic vectors | 2.8.3.10). The transferase belongs to |
| | | for controlled expression of RNA | the citX family (Schneider et al., |
| | | and/or protein, fusion protein | Biochem. 39: 9438-9450, 2000). |
| | | production, genetic modification, | Carbohydrate metabolism impacts on |
| | | mutagenesis amplification of | flavor, functionality and survival as |
| | | genetic material or for other | well as growth. |
| | | genetic or protein manipulations. | |
| | | Altered survival characteristics: | |
| | | (survival of industrial processes, | |
| | | growth or storage in product | |
| | | formats, persistence in gut | |
| | | environment). | |
| | | Modified carbohydrate levels or | |
| | | functional properties. | |
| | | Altered metabolic properties. | |
| | | Modified citrate metabolism. | |
| | | Altered probiotic attributes. | |
| | | Organisms or materials with | |
| | | improved health properties | |
| | | (including immunoregulatory, | |
| | | anticancer, gut health). | |
| | | Improved fermentation properties | |
| | | or other industrially useful | |
| | | processes. | |
| 64 | 158 | Removal of undesirable flavor | Homologue of pycB, encoding |
| | | characteristics. | pyruvate carboxylase, which |
| | | Modified flavor, aroma, texture | catalyzes a two-step reaction, |
| | | attributes. | involving the ATP-dependent |
| | | Construction of genetic vectors | carboxylation of the covalently |
| | | for controlled expression of RNA | attached biotin in the first step and |
| | | and/or protein, fusion protein | the transfer of the carboxyl group to |
| | | production, genetic modification, | pyruvate to generate oxaloacetate in |
| | | mutagenesis amplification of | the second. The enzyme is involved |
| | | genetic material or for other | in gluconeogenesis and amino acid |
| | | genetic or protein manipulations. | biotransformations. Carbohydrate |
| | | Altered survival characteristics: | metabolism impacts on flavor, |
| | | (survival of industrial processes, | functionality and survival as well as |
| | | growth or storage in product | growth. |
| | | formats, persistence in gut | |
| | | environment). | |
| | | Modified carbohydrate levels or | |
| | | functional properties. | |
| | | Altered metabolic properties. | |
| | | Altered probiotic attributes. | |
| | | Organisms or materials with | |
| | | improved health properties | |
| | | (including immunoregulatory, | |
| | | anticancer, gut health) | |
| | | Improved fermentation properties | |
| | | or other industrially useful | |
| | | processes. | |
| 66 | 160 | Production of desirable flavors. | Homologue of fox3, encoding a 3- |
| | | Modified flavor, aroma and/or | oxoacyl-coenzyme A thiolase (EC |
| | | texture attributes. | 2.3.1.16) also called acetyl-CoA C- |
| | | Construction of genetic vectors | acyltransferase, which participates in |
| | | for controlled expression of RNA | the beta-oxidation of fatty acids. |
| | | and/or protein, fusion protein | Expression of the FOX3 gene can be |
| | | production, genetic modification, | induced by oleate and repressed by |
| | | mutagenesis amplification of | glucose (Einerhand et al., Mol. Cell. |
| | | genetic material or for other | Biol. 15: 3405-3414, 1995). Free fatty |
| | | genetic or protein manipulations. | acids are important in providing |
| | | Altered survival characteristics: | flavor-bearing compounds for dairy |
| | | survival of industrial processes, | products such as cheese, and have a |
| | | growth or storage in product | significant role in both flavor and |
| | | formats, persistence in gut | texture. Used extensively in wide |
| | | environment. | range of convenience foods. Short |
| | | Altered metabolic properties. | chain fatty acids are known to have a |
| | | Modified lipid, glycolipid or free | variety of health impacts. |
| | | fatty acid levels or functional | |
| | | properties. | |
| | | Modified production of short | |
| | | chain fatty acids. | |
| | | Altered lipid metabolism. | |
| | | Altered probiotic attributes. | |
| | | Organisms or materials with | |
| | | improved health properties | |
| | | (including immunoregulatory, | |
| | | anticancer, gut health). | |
| | | Improved fermentation properties | |
| | | or other industrially useful | |
| | | processes. | |
| 67 | 161 | Construction of genetic vectors | Homologue of YchH, encoding an |
| | | for controlled expression of RNA | acetyltransferase. Transfer of acetyl |
| | | and/or protein, fusion protein | groups are important in regulation of |
| | | production, genetic modification, | metabolic pathways was well as co- |
| | | mutagenesis amplification of | factor production and can influence |
| | | genetic material or for other | flavor and/or functionality. |
| | | genetic or protein manipulations. | |
| | | Production of desirable flavors. | |
| | | Modified flavor, aroma and/or | |
| | | texture attributes. | |
| | | Altered survival characteristics: | |
| | | survival of industrial processes, | |
| | | growth or storage in product | |
| | | formats, persistence in gut | |
| | | environment. | |
| | | Altered viability in response to | |
| | | stress conditions. | |
| | | Altered metabolic properties or | |
| | | regulation of metabolic | |
| | | pathways. | |
| | | Altered probiotic attributes. | |
| 68 | 162 | Production of desirable flavors. | Homologue of SC6F7, encoding a |
| | | Modified flavor, aroma and/or | lipase. Lipases are involved in the |
| | | texture attributes. | breakdown of triglycerides, |
| | | Construction of genetic vectors | metabolism, growth, production of |
| | | for controlled expression of RNA | flavor compounds, and the release of |
| | | and/or protein, fusion protein | free fatty acids. Can also catalyze |
| | | production, genetic modification, | esterification of glycerol to form |
| | | mutagenesis amplification of | mono, di- and triglycerides. Free fatty |
| | | genetic material or for other | acids are important in providing |
| | | genetic or protein manipulations. | flavor-bearing compounds for dairy |
| | | Altered survival characteristics: | products such as cheese, and have a |
| | | survival of industrial processes, | significant role in both flavor and |
| | | growth or storage in product | texture. Used extensively in wide |
| | | formats, persistence in gut | range of convenience foods. Short |
| | | environment. | chain fatty acids are known to have a |
| | | Altered metabolic properties. | variety of health impacts. |
| | | Modified lipid, glycolipid or free | |
| | | fatty acid levels or functional | |
| | | properties. | |
| | | Modified production of short | |
| | | chain fatty acids. | |
| | | Altered lipid metabolism. | |
| | | Altered probiotic attributes. | |
| | | Organisms or materials with | |
| | | improved health properties | |
| | | (including immunoregulatory, | |
| | | anticancer, gut health). | |
| | | Improved fermentation properties | |
| | | or other industrially useful | |
| | | processes. | |
| 69 | 163 | Altered cell wall or cell surface | Homologue of yohH, a |
| | | characteristics, structures or | lipopolysaccharide synthesis protein. |
| | | functions. | YohH is involved in techoic acid |
| | | Modified adhesion to human or | synthesis, and important for cell wall |
| | | animal cells or cell lines. | functions including adhesion, |
| | | Production of desirable flavors. | immune cell interaction, product |
| | | Modified flavor, aroma and/or | texture. |
| | | texture attributes. | |
| | | Construction of genetic vectors | |
| | | for controlled expression of RNA | |
| | | and/or protein, fusion protein | |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| | | Improved fermentation properties | |
| | | or other industrially useful | |
| | | processes. | |
| 69 | 164 | Altered cell wall or cell surface | Homologue of yohJ, encoding a |
| | | characteristics, structures or | lipopolysaccharide biosynthesis |
| | | functions. | protein. YohJ is involved in techoic |
| | | Modified adhesion to human or | acid synthesis, and important for cell |
| | | animal cells or cell lines. | wall functions including adhesion, |
| | | Production of desirable flavors. | immune cell interaction and product |
| | | Modified flavor, aroma and/or | texture. |
| | | texture attributes. | |
| | | Construction of genetic vectors | |
| | | for controlled expression of RNA | |
| | | and/or protein, fusion protein | |
| | | production, genetic modification, | |
| | | mutagenesis amplification of | |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved antimicrobial | |
| | | properties. | |
| | | Improved fermentation properties | |
| | | or other industrially useful | |
| | | processes. | |
| 70 | 165 | Construction of genetic vectors | Homologue of pstS, encoding a |
| | | for controlled expression of RNA | phosphate-binding protein that is part |
| | | and/or protein, fusion protein | of the phosphate specific transporter |
| | | production, genetic modification, | (Pst) in bacteria. Pst is a multisubunit |
| | | mutagenesis amplification of | system and belongs to the ABC |
| | | genetic material or for other | superfamily of transporters (Novak et |
| | | genetic or protein manipulations. | al., J Bacteriol. 181: 1126-1133, |
| | | Production of desirable flavors. | 1999). Intracellular phosphate levels |
| | | Modified flavor, aroma and/or | influence survival of bacteria in |
| | | texture attributes. | environmental stress conditions, and |
| | | Altered survival characteristics: | are involved in the stringent response. |
| | | survival of industrial processes, | |
| | | growth or storage in product | |
| | | formats, persistence in gut | |
| | | environment. | |
| | | Altered viability in response to | |
| | | stress conditions. | |
| | | Altered metabolic properties or | |
| | | regulation of metabolic | |
| | | pathways. | |
| | | Altered probiotic attributes. | |
| 71 | 166 | Altered cell wall or cell surface | Homologue of oppA, encoding an |
| | | characteristics, structures or | integral membrane protein of the |
| | | functions. | oligopeptide transport system (Opp) |
| | | Production of bioactive or | of Lactococcus lactis. Opp proteins |
| | | functional polypeptides. | and the proteinase PrtP are important |
| | | Modified adhesion to human or | components of the proteolytic system. |
| | | animal cells or cell lines. | The Opp system belongs to the |
| | | Altered amino acid metabolism. | superfamily of ABC transporters and |
| | | Removal of undesirable flavor | consists of five proteins: the integral |
| | | characteristics. | membrane proteins OppB and OppC, |
| | | Production of desirable flavors. | the ATP-binding proteins OppD and |
| | | Modified flavor, aroma and/or | OppF, and the OppA a receptor |
| | | texture attributes. | protein (Detmers et al., Proc. Natl. |
| | | Construction of genetic vectors | Acad. Sci. USA 97: 12487-12492, |
| | | for controlled expression of RNA | 2000). Important for the uptake and |
| | | and/or protein, fusion protein | supply of amino acids to bacteria, and |
| | | production, genetic modification, | the resultant production of flavorful |
| | | mutagenesis amplification of | or functional amino-acid degradation |
| | | genetic material or for other | products. |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Improved fermentation properties | |
| | | or other industrially useful | |
| | | processes. | |
| 71 | 167 | Altered cell wall or cell surface | Homologue of oppB, encoding an |
| | | characteristics, structures or | integral membrane protein of the |
| | | functions. | oligopeptide transport system (Opp) |
| | | Production of bioactive or | of Lactococcus lactis. Opp proteins |
| | | functional polypeptides. | and the proteinase PrtP are important |
| | | Modified adhesion to human or | components of the proteolytic system. |
| | | animal cells or cell lines. | The Opp system belongs to the |
| | | Altered amino acid metabolism. | superfamily of ABC transporters and |
| | | Removal of undesirable flavor | consists of five proteins: the integral |
| | | characteristics. | membrane proteins OppB and OppC, |
| | | Production of desirable flavors. | the ATP-binding proteins OppD and |
| | | Modified flavor, aroma and/or | OppF, and the OppA a receptor |
| | | texture attributes. | protein (Detmers et al., Proc. Natl. |
| | | Construction of genetic vectors | Acad. Sci. USA 97: 12487-12492, |
| | | for controlled expression of RNA | 2000). Important for the uptake and |
| | | and/or protein, fusion protein | supply of amino acids to bacteria, and |
| | | production, genetic modification, | the resultant production of flavorful |
| | | mutagenesis amplification of | or functional amino-acid degradation |
| | | genetic material or for other | products. |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Improved fermentation properties | |
| | | or other industrially useful | |
| | | processes. | |
| 71 | 168 | Altered cell wall or cell surface | Homologue of OppC, encoding an |
| | | characteristics, structures or | integral membrane protein of the |
| | | functions. | oligopeptide transport system (Opp) |
| | | Production of bioactive or | of Lactococcus lactis. Opp proteins |
| | | functional polypeptides. | and the proteinase PrtP are important |
| | | Modified adhesion to human or | components of the proteolytic system. |
| | | animal cells or cell lines. | The Opp system belongs to the |
| | | Altered amino acid metabolism. | superfamily of ABC transporters and |
| | | Removal of undesirable flavor | consists of five proteins: the integral |
| | | characteristics. | membrane proteins OppB and OppC, |
| | | Production of desirable flavors. | the ATP-binding proteins OppD and |
| | | Modified flavor, aroma and/or | OppF, and the OppA a receptor |
| | | texture attributes. | protein (Detmers et al., Proc. Natl. |
| | | Construction of genetic vectors | Acad. Sci. USA 97: 12487-12492, |
| | | for controlled expression of RNA | 2000). Important for the uptake and |
| | | and/or protein, fusion protein | supply of amino acids to bacteria, and |
| | | production, genetic modification, | the resultant production of flavorful |
| | | mutagenesis amplification of | or functional amino-acid degradation |
| | | genetic material or for other | products. |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Improved fermentation properties | |
| | | or other industrially useful | |
| | | processes. | |
| 71 | 169 | Altered cell wall or cell surface | Homologue of OppF, encoding an |
| | | characteristics, structures or | integral membrane protein of the |
| | | functions. | oligopeptide transport system (Opp) |
| | | Production of bioactive or | of Lactococcus lactis. Opp proteins |
| | | functional polypeptides. | and the proteinase PrtP are important |
| | | Modified adhesion to human or | components of the proteolytic system. |
| | | animal cells or cell lines. | The Opp system belongs to the |
| | | Altered amino acid metabolism. | superfamily of ABC transporters and |
| | | Removal of undesirable flavor | consists of five proteins: the integral |
| | | characteristics. | membrane proteins OppB and OppC, |
| | | Production of desirable flavors. | the ATP-binding proteins OppD and |
| | | Modified flavor, aroma and/or | OppF, and the OppA a receptor |
| | | texture attributes. | protein (Detmers et al., Proc. Natl. |
| | | Construction of genetic vectors | Acad. Sci. USA 97: 12487-12492, |
| | | for controlled expression of RNA | 2000). Important for the uptake and |
| | | and/or protein, fusion protein | supply of amino acids to bacteria, and |
| | | production, genetic modification, | the resultant production of flavorful |
| | | mutagenesis amplification of | or functional amino-acid degradation |
| | | genetic material or for other | products. |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Improved fermentation properties | |
| | | or other industrially useful | |
| | | processes. | |
| 72 | 170 | Removal of undesirable flavor | Homologue of thdF, which is |
| | | characteristics. | involved in the oxidation of |
| | | Modified flavor, aroma, texture | derivatives of the sulphur-containing |
| | | attributes. | heterocycle thiophene and is induced |
| | | Construction of genetic vectors | during stationary phase. The thdF |
| | | for controlled expression of RNA | gene is subject to substantial |
| | | and/or protein, fusion protein | catabolite repression by glucose and |
| | | production, genetic modification, | its expression is also greatly |
| | | mutagenesis amplification of | decreased in the absence of oxygen |
| | | genetic material or for other | (Zabel et al., Microbios. 101: 89-103, |
| | | genetic or protein manipulations. | 2000). |
| | | Altered survival characteristics: | |
| | | (survival of industrial processes, | |
| | | growth or storage in product | |
| | | formats, persistence in gut | |
| | | environment). | |
| | | Altered metabolic properties. | |
| | | Modified metabolism of sulphur- | |
| | | containing compounds. | |
| | | Altered probiotic attributes. | |
| | | Organisms or materials with | |
| | | improved health properties | |
| | | (including immunoregulatory, | |
| | | anticancer, gut health). | |
| 72 | 171 | Construction of genetic vectors | Homologue of gidA, encoding a |
| | | for controlled expression of RNA | glucose-inhibited division protein A, |
| | | and/or protein, fusion protein | which is involved in cell division and |
| | | production, genetic modification, | in moderating translational fidelity |
| | | mutagenesis amplification of | (Kinscherf and Willis, J. Bacteriol. |
| | | genetic material or for other | 184: 2281-2286, 2002). Affects |
| | | genetic or protein manipulations. | growth and viability in different |
| | | Production of desirable flavors. | growth environments. |
| | | Modified flavor, aroma and/or | |
| | | texture attributes. | |
| | | Altered survival characteristics: | |
| | | survival of industrial processes, | |
| | | growth or storage in product | |
| | | formats, persistence in gut | |
| | | environment. | |
| | | Altered viability in response to | |
| | | stress conditions. | |
| | | Altered metabolic properties or | |
| | | regulation of metabolic | |
| | | pathways. | |
| | | Altered probiotic attributes. | |
| 73 | 172 | Construction of genetic vectors | Homologue of poxB, encoding a |
| | | for controlled expression of RNA | pyruvate oxidase (EC 1.2.3.3), which |
| | | and/or protein, fusion protein | decarboxylates pyruvate. The enzyme |
| | | production, genetic modification, | is a flavoprotein (FAD) requiring |
| | | mutagenesis amplification of | thiamine diphosphate and is |
| | | genetic material or for other | important for aerobic growth and |
| | | genetic or protein manipulations. | survival in aerobic conditions. |
| | | Production of desirable flavors. | Carbohydrate metabolism impacts on |
| | | Modified flavor, aroma and/or | flavor, functionality and survival as |
| | | texture attributes. | well as growth. |
| | | Altered survival characteristics: | |
| | | survival of industrial processes, | |
| | | growth or storage in product | |
| | | formats, persistence in gut | |
| | | environment. | |
| | | Altered viability in response to | |
| | | stress conditions. | |
| | | Altered metabolic properties or | |
| | | regulation of metabolic | |
| | | pathways. | |
| | | Altered probiotic attributes. | |
| 74 | 173 | Altered amino acid metabolism. | Homologue of gltD, encoding a |
| | | Removal of undesirable flavor | glutamate synthase (EC 1.4.1.13), |
| | | characteristics. | which catalyzes the reductive transfer |
| | | Production of desirable flavors. | of the amide group of glutamine to |
| | | Modified flavor, aroma and/or | the keto position of 2-oxoglutarate to |
| | | texture attributes. | yield two molecules of glutamate. |
| | | Construction of genetic vectors | The resulting glutamine and |
| | | for controlled expression of RNA | glutamate serve as nitrogen donors in |
| | | and/or protein, fusion protein | the biosynthesis of various nitrogen- |
| | | production, genetic modification, | containing compounds. This pathway |
| | | mutagenesis amplification of | is involved in the integration of |
| | | genetic material or for other | carbon and nitrogen assimilations. |
| | | genetic or protein manipulations. | Amino acid degradation products |
| | | Altered survival characteristics: | greatly contribute to flavor or to off- |
| | | survival of industrial processes, | flavors (Rijnen et al., Appl. Environ. |
| | | growth or storage in product | Microbiol. 65: 4873-4880, 1999). |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 75 | 174 | Altered amino acid metabolism. | Homologue of dhfR, encoding a |
| | | Removal of undesirable flavor | dihydrofolate reductase (EC 1.5.1.3), |
| | | characteristics. | which catalyzes the essential step for |
| | | Production of desirable flavors. | de novo glycine and purine synthesis, |
| | | Modified flavor, aroma and/or | DNA precursor synthesis, and for the |
| | | texture attributes. | conversion of dUMP to dTMP. |
| | | Construction of genetic vectors | Involved in folate production, which |
| | | for controlled expression of RNA | has major health impacts and also |
| | | and/or protein, fusion protein | detoxifies some chemotherapeutic |
| | | production, genetic modification, | drugs and other cytotoxic |
| | | mutagenesis amplification of | compounds. |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Increased folate production. | |
| | | Altered probiotic attributes. | |
| | | Modified health properties | |
| | | (including immunoregulatory, | |
| | | anticancer, gut health). | |
| | | Protection of intestinal cells from | |
| | | toxic compounds. | |
| | | Modified antibiotic resistance. | |
| | | Improved fermentation properties | |
| | | or other industrially useful | |
| | | processes. | |
| 77 | 176 | Altered amino acid metabolism. | Homologue of trpA, encoding the |
| | | Removal of undesirable flavor | tryptophan synthase alpha chain (EC |
| | | characteristics. | 4.2.1.20). TrpA catalyzes the |
| | | Production of desirable flavors. | formation of indole from the cleavage |
| | | Modified flavor, aroma and/or | of 3-indolyl-D-glyceraldehyde 3′- |
| | | texture attributes. | phosphate. Seven structural genes are |
| | | Construction of genetic vectors | required for tryptophan biosynthesis: |
| | | for controlled expression of RNA | trpABCDEFG. TrpA encodes the |
| | | and/or protein, fusion protein | tryptophan synthase alpha chain (EC |
| | | production, genetic modification, | 4.2.1.20) Tryptophan is important for |
| | | mutagenesis amplification of | flavor development. Amino acid |
| | | genetic material or for other | degradation products greatly |
| | | genetic or protein manipulations. | contribute to flavor or to off-flavors |
| | | Altered survival characteristics: | (Rijnen et al., Appl. Environ. |
| | | survival of industrial processes, | Microbiol. 65: 4873-4880, 1999). |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 77 | 177 | Altered amino acid metabolism. | Homologue of trpB, encoding |
| | | Removal of undesirable flavor | tryptophan synthase beta chain (EC |
| | | characteristics. | 4.2.1.20). TrpB catalyzes the |
| | | Production of desirable flavors. | condensation of indole to a serine- |
| | | Modified flavor, aroma and/or | derived aminoacrylate moiety bound |
| | | texture attributes. | to pyridoxal phosphate. Seven |
| | | Construction of genetic vectors | structural genes are required for |
| | | for controlled expression of RNA | tryptophan biosynthesis: |
| | | and/or protein, fusion protein | trpABCDEFG. TrpB encodes the |
| | | production, genetic modification, | tryptophan synthase beta chain (EC |
| | | mutagenesis amplification of | 4.2.1.20). Tryptophan is important for |
| | | genetic material or for other | flavor development. Amino acid |
| | | genetic or protein manipulations. | degradation products greatly |
| | | Altered survival characteristics: | contribute to flavor or to off-flavors |
| | | survival of industrial processes, | (Rijnen et al., Appl. Environ. |
| | | growth or storage in product | Microbiol. 65: 4873-4880, 1999). |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 77 | 178 | Altered amino acid metabolism. | Homologue of trpC, encoding the |
| | | Removal of undesirable flavor | bifunctional enzyme |
| | | characteristics. | phosphoribosylanthranilate isomerase |
| | | Production of desirable flavors. | (EC 5.3.1.24)-indoleglycerol |
| | | Modified flavor, aroma and/or | phosphate synthetase (EC 4.1.1.48). |
| | | texture attributes. | TrpC catalyzes the ring closure of 1- |
| | | Construction of genetic vectors | (2-carboxyphenylamino)-1- |
| | | for controlled expression of RNA | deoxyribulose 5′-phosphate to |
| | | and/or protein, fusion protein | indoleglycerol phosphate, the fifth |
| | | production, genetic modification, | step in the pathway of tryptophan |
| | | mutagenesis amplification of | biosynthesis from chorismate. Seven |
| | | genetic material or for other | structural genes are required for |
| | | genetic or protein manipulations. | tryptophan biosynthesis: |
| | | Altered survival characteristics: | trpABCDEFG. Tryptophan is |
| | | survival of industrial processes, | important for flavor development. |
| | | growth or storage in product | Amino acid degradation products |
| | | formats, persistence in gut | greatly contribute to flavor or to off- |
| | | environment. | flavors (Rijnen et al., Appl. Environ. |
| | | Altered metabolic properties. | Microbiol. 65: 4873-4880, 1999). |
| | | Altered probiotic attributes. | |
| | | Modified health properties | |
| | | (including immunoregulatory, | |
| | | anticancer, gut health). | |
| | | Modified antibiotic resistance. | |
| 77 | 179 | Altered amino acid metabolism. | Homologue of trpD, encoding |
| | | Removal of undesirable flavor | phosphoribosyl anthranilate |
| | | characteristics. | tranferase (EC 2.4.2.18),. TrpD |
| | | Production of desirable flavors. | catalyzes the reaction N-(5-phospho- |
| | | Modified flavor, aroma and/or | D-ribosyl)-anthranilate + diphosphate = anthranilate + 5- |
| | | texture attributes. | phospo-a-D- |
| | | Construction of genetic vectors | ribose 1-diphosphate. Seven |
| | | for controlled expression of RNA | structural genes are required for |
| | | and/or protein, fusion protein | tryptophan biosynthesis: |
| | | production, genetic modification, | trpABCDEFG. Tryptophan is |
| | | mutagenesis amplification of | important for flavor development. |
| | | genetic material or for other | Amino acid degradation products |
| | | genetic or protein manipulations. | greatly contribute to flavor or to off- |
| | | Altered survival characteristics: | flavors (Rijnen et al., Appl. Environ. |
| | | survival of industrial processes, | Microbiol. 65: 4873-4880, 1999). |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 77 | 180 | Altered amino acid metabolism. | Homologue of trpF, encoding |
| | | Removal of undesirable flavor | phosphoribosylanthranilate isomerase |
| | | characteristics. | (EC 5.3.1.24). TrpF catalyzes the |
| | | Production of desirable flavors. | conversion of N-(5′- |
| | | Modified flavor, aroma and/or | diphosphoribosyl)anthranilate to 1- |
| | | texture attributes. | (o-carboxyphenylamino)-1- |
| | | Construction of genetic vectors | deoxyribulose 5 phosphate. Seven |
| | | for controlled expression of RNA | structural genes are required for |
| | | and/or protein, fusion protein | tryptophan biosynthesis: |
| | | production, genetic modification, | trpABCDEFG. Tryptophan is |
| | | mutagenesis amplification of | important for flavor development. |
| | | genetic material or for other | Amino acid degradation products |
| | | genetic or protein manipulations. | greatly contribute to flavor or to off- |
| | | Altered survival characteristics: | flavors (Rijnen et al., Appl. Environ. |
| | | survival of industrial processes, | Microbiol. 65: 4873-4880, 1999). |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| 78 | 181 | Construction of genetic vectors | Homologue of purine nucleoside |
| | | for controlled expression of RNA | phosphorylase (PNP) (EC 2.4.2.1). |
| | | and/or protein, fusion protein | PNP catalyzes the reversible |
| | | production, genetic modification, | phosphorolysis of (2′-deoxy)purine |
| | | mutagenesis amplification of | ribonucleosides to free base and (2′- |
| | | genetic material or for other | deoxy)ribose-1-phosphate and has a |
| | | genetic or protein manipulations. | metabolic role in purine salvage. |
| | | Production of desirable flavors. | Intracellular phosphate levels |
| | | Modified flavor, aroma and/or | influence survival of bacteria in |
| | | texture attributes. | environmental stress conditions, and |
| | | Altered survival characteristics: | are involved in the stringent response. |
| | | survival of industrial processes, | |
| | | growth or storage in product | |
| | | formats, persistence in gut | |
| | | environment. | |
| | | Altered viability in response to | |
| | | stress conditions. | |
| | | Altered metabolic properties or | |
| | | regulation of metabolic | |
| | | pathways. | |
| | | Altered probiotic attributes. | |
| 79 | 182 | Construction of genetic vectors | Homologue to relA. RelA plays a role |
| | | for controlled expression of RNA | in synthesis and degradation of the |
| | | and/or protein, fusion protein | highly phosphorylated guanosine |
| | | production, genetic modification, | nucleotides (p)ppGp. Intracellular |
| | | mutagenesis amplification of | phosphate levels influence survival of |
| | | genetic material or for other | bacteria in environmental stress |
| | | genetic or protein manipulations. | conditions, and are involved in the |
| | | Production of desirable flavors. | stringent response. |
| | | Modified flavor, aroma and/or | |
| | | texture attributes. | |
| | | Altered survival characteristics: | |
| | | survival of industrial processes, | |
| | | growth or storage in product | |
| | | formats, persistence in gut | |
| | | environment. | |
| | | Altered viability in response to | |
| | | stress conditions. | |
| | | Altered metabolic properties or | |
| | | regulation of metabolic | |
| | | pathways. | |
| | | Altered probiotic attributes. | |
| 80 | 183 | Altered cell wall or cell surface | Homologue of lysostaphin, an |
| | | characteristics, structures or | antimicrobial immunity factor of |
| | | functions. | Staphylococcus simulans biovar |
| | | Improved antimicrobial | staphylolyticus active against |
| | | properties | Staphylococcus aureas. Lysostaphin |
| | | Modified adhesion to human or | is currently being investigated for use |
| | | animal cells or cell lines. | against mastitis in dairy cattle, caused |
| | | Production of desirable flavors. | by Staphylococcus aureus (Kerr et |
| | | Modified flavor, aroma and/or | al., Nat. Biotechnol. 19: 66-70, 2001). |
| | | texture attributes. | The gene contains the conserved |
| | | Construction of genetic vectors | motif GPHLHF, which is also present |
| | | for controlled expression of RNA | in several secreted peptidases. |
| | | and/or protein, fusion protein | Lysostaphin has utility as an |
| | | production, genetic modification, | antimicrobial for human and |
| | | mutagenesis amplification of | veterinary use. |
| | | genetic material or for other | |
| | | genetic or protein manipulations. | |
| | | Altered survival characteristics: | |
| | | survival of industrial processes, | |
| | | growth or storage in product | |
| | | formats, persistence in gut | |
| | | environment. | |
| | | Altered metabolic properties. | |
| | | Altered probiotic attributes. | |
| | | Modified health properties | |
| | | (including immunoregulatory, | |
| | | anticancer, gut health). | |
| | | Modified antibiotic resistance. | |
| | | Improved fermentation properties | |
| | | or other industrially useful | |
| | | processes. | |

Isolated polynucleotides of the present invention include the polynucleotides identified herein as SEQ ID NOS: 1-80; isolated polynucleotides comprising a polynucleotide sequence selected from the group consisting of SEQ ID NOS: 1-80; isolated polynucleotides comprising at least a specified number of contiguous residues (x-mers) of any of the polynucleotides identified as SEQ ID NOS: 1-80; isolated polynucleotides comprising a polynucleotide sequence that is complementary to any of the above polynucleotides; isolated polynucleotides comprising a polynucleotide sequence that is a reverse sequence or a reverse complement of any of the above polynucleotides; antisense sequences corresponding to any of the above polynucleotides; and variants of any of the above polynucleotides, as that term is described in this specification.

The word “polynucleotide(s),” as used herein, means a single or double stranded polymer of deoxyribonucleotide or ribonucleotide bases and includes DNA and corresponding RNA molecules, including mRNA molecules, both sense and antisense strands of DNA and RNA molecules, and comprehends cDNA, genomic DNA and recombinant DNA, as well as wholly or partially synthesized polynucleotides. A polynucleotide of the present invention may be an entire gene, or any portion thereof. A gene is a DNA sequence which codes for a functional protein or RNA molecule. Operable antisense polynucleotides may comprise a fragment of the corresponding polynucleotide, and the definition of “polynucleotide” therefore includes all operable antisense fragments. Antisense polynucleotides and techniques involving antisense polynucleotides are well known in the art and are described, for example, in Robinson-Benion, et al., “Antisense techniques,” Methods in Enzymol. 254(23): 363-375, 1995; and Kawasaki, et al., Artific. Organs 20 (8): 836-848, 1996.

The definitions of the terms “complement,” “reverse complement,” and “reverse sequence,” as used herein, are best illustrated by the following examples. For the sequence 5′ AGGACC 3′, the complement, reverse complement, and reverse sequences are as follows:

| | |
|---|---|
| complement | 3′ TCCTGG 5′ |
| reverse complement | 3′ GGTCCT 5′ |
| reverse sequence | 5′ CCAGGA 3′ |

Identification of genomic DNA and heterologous species DNA can be accomplished by standard DNA/DNA hybridization techniques, under appropriately stringent conditions, using all or part of a DNA sequence as a probe to screen an appropriate library. Alternatively, PCR techniques using oligonucleotide primers that are designed based on known DNA and protein sequences can be used to amplify and identify other identical or similar DNA sequences. Synthetic DNA corresponding to the identified sequences or variants thereof may be produced by conventional synthesis methods. All of the polynucleotides described herein are isolated and purified, as those terms are commonly used in the art.

The polynucleotides identified as SEQ ID NOS: 1-80 contain open reading frames (“ORFs”), or partial open reading frames, encoding polypeptides. Additionally, polynucleotides identified as SEQ ID NOS: 1-80 may contain non-coding sequences such as promoters and terminators that may be useful as control elements. Additionally, open reading frames encoding polypeptides may be identified in extended or full-length sequences corresponding to the sequences set out as SEQ ID NOS: 81-183. Open reading frames may be identified using techniques that are well known in the art. These techniques include, for example, analysis for the location of known start and stop codons, most likely reading frame identification based on codon frequencies, similarity to known bacterial expressed genes, etc. Tools and software suitable for ORF analysis include GeneWise (The Sanger Center, Wellcome Trust Genome Campus, Hinxton, Cambridge CB10 1SA, United Kingdom), Diogenes (Computational Biology Centers, University of Minnesota, Academic Health Center, UMHG Box 43 Minneapolis Minn. 55455), and GRAIL (Informatics Group, Oak Ridge National Laboratories, Oak Ridge, Tenn., TN). Open reading frames and portions of open reading frames may be identified in the polynucleotides of the present invention. Once a partial open reading frame is identified, the polynucleotide may be extended in the area of the partial open reading frame using techniques that are well known in the art until the polynucleotide for the full open reading frame is identified. Thus, polynucleotides and open reading frames encoding polypeptides may be identified using the polynucleotides of the present invention.

Once open reading frames are identified in the polynucleotides of the present invention, the open reading frames may be isolated and/or synthesized. Expressible genetic constructs comprising the open reading frames and suitable promoters, initiators, terminators, etc., which are well known in the art, may then be constructed. Such genetic constructs may be introduced into a host cell to express the polypeptide encoded by the open reading frame. Suitable host cells may include various prokaryotic and eukaryotic cells. In vitro expression of polypeptides is also possible, as well known in the art.

As used herein, the term “oligonucleotide” refers to a relatively short segment of a polynucleotide sequence, generally comprising between 6 and 60 nucleotides, and comprehends both probes for use in hybridization assays and primers for use in the amplification of DNA by polymerase chain reaction.

As used herein, the term “x-mer,” with reference to a specific value of “x,” refers to a polynucleotide comprising at least a specified number (“x”) of contiguous residues of any of the polynucleotides identified as SEQ ID NOS: 1-80. The value of x may be from about 20 to about 600, depending upon the specific sequence.

In another aspect, the present invention provides isolated polypeptides encoded, or partially encoded, by the above polynucleotides. In specific embodiments, such polypeptides comprise a sequence selected from the group consisting of SEQ ID NO: 81-183, and variants thereof. As used herein, the term “polypeptide” encompasses amino acid chains of any length, including full-length proteins, wherein the amino acid residues are linked by covalent peptide bonds. The term “polypeptide encoded by a polynucleotide” as used herein, includes polypeptides encoded by a polynucleotide which comprises an isolated polynucleotide sequence or variant provided herein. Polypeptides of the present invention may be naturally purified products, or may be produced partially or wholly using recombinant techniques. Such polypeptides may be glycosylated with bacterial, fungal, mammalian or other eukaryotic carbohydrates or may be non-glycosylated.

Polypeptides of the present invention may be produced recombinantly by inserting a polynucleotide that encodes the polypeptide into an expression vector and expressing the polypeptide in an appropriate host. Any of a variety of expression vectors known to those of ordinary skill in the art may be employed. Expression may be achieved in any appropriate host cell that has been transformed or transfected with an expression vector containing a polypeptide encoding a recombinant polypeptide. Suitable host cells include prokaryotes, yeast and higher eukaryotic cells. Preferably, the host cells employed are Escherichia coli, Lactococcus lactis, Lactobacillus, insect, yeast or a mammalian cell line such as COS or CHO. The polynucleotide(s) expressed in this manner may encode naturally occurring polypeptides, portions of naturally occurring polypeptides, or other variants thereof.

In a related aspect, polypeptides are provided that comprise at least a functional portion of a polypeptide having an amino acid sequence encoded by a polynucleotide of the present invention. As used herein, a “functional portion” of a polypeptide is that portion which contains the active site essential for affecting the function of the polypeptide, for example, the portion of the molecule that is capable of binding one or more reactants. The active site may be made up of separate portions present on one or more polypeptide chains and will generally exhibit high binding affinity.

Functional portions of a polypeptide may be identified by first preparing fragments of the polypeptide by either chemical or enzymatic digestion of the polypeptide, or by mutation analysis of the polynucleotide that encodes the polypeptide and subsequent expression of the resulting mutant polypeptides. The polypeptide fragments or mutant polypeptides are then tested to determine which portions retain biological activity, using, for example, the representative assays provided below.

Portions and other variants of the inventive polypeptides may be generated by synthetic or recombinant means. Synthetic polypeptides having fewer than about 100 amino acids, and generally fewer than about 50 amino acids, may be generated using techniques that are well known to those of ordinary skill in the art. For example, such polypeptides may be synthesized using any of the commercially available solid-phase techniques, such as the Merrifield solid-phase synthesis method, where amino acids are sequentially added to a growing amino acid chain (See Merrifield, J. Am. Chem. Soc. 85:2149-2154, 1963). Equipment for automated synthesis of polypeptides is commercially available from suppliers such as Perkin Elmer/Applied Biosystems, Inc. (Foster City, Calif.), and may be operated according to the manufacturer's instructions. Variants of a native polypeptide may be prepared using standard mutagenesis techniques, such as oligonucleotide-directed site-specific mutagenesis (Kunkel, Proc. Natl. Acad. Sci. USA 82: 488-492, 1985). Sections of DNA sequences may also be removed using standard techniques to permit preparation of truncated polypeptides.

In general, the polypeptides disclosed herein are prepared in an isolated, substantially pure form. Preferably, the polypeptides are at least about 80% pure; more preferably at least about 90% pure; and most preferably at least about 99% pure.

As used herein, the term “variant” comprehends polynucleotide or polypeptide sequences different from the specifically identified sequences, wherein one or more nucleotides or amino acid residues is deleted, substituted, or added. Variants may be naturally occurring allelic variants, or non-naturally occurring variants. Variant polynucleotide sequences preferably exhibit at least 60%, more preferably at least 75%, more preferably yet at least 90%, and most preferably at least 95% identity to a sequence of the present invention. Variant polypeptide sequences preferably exhibit at least 60%, more preferably at least 75%, more preferably yet at least 90%, and most preferably at least 95% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100.

Polynucleotide and polypeptide sequences may be aligned, and the percentage of identical residues in a specified region may be determined against another polynucleotide or polypeptide, using computer algorithms that are publicly available. Two exemplary algorithms for aligning and identifying the similarity of polynucleotide sequences are the BLASTN and FASTA algorithms. Polynucleotides may also be analyzed using the BLASTX algorithm, which compares the six-frame conceptual translation products of a nucleotide query sequence (both strands) against a protein sequence database. The percentage identity of polypeptide sequences may be examined using the BLASTP algorithm. The BLASTN, BLASTX and BLASTP programs are available on the NCBI anonymous FTP server and from the National Center for Biotechnology Information (NCBI), National Library of Medicine, Building 38A, Room 8N805, Bethesda, Md. 20894, USA. The BLASTN algorithm Version 2.0.4 [Feb. 24, 1998], Version 2.0.6 [Sep. 16, 1998] and Version 2.0.11 [Jan. 20, 2000], set to the parameters described below, is preferred for use in the determination of polynucleotide variants according to the present invention. The BLASTP algorithm, set to the parameters described below, is preferred for use in the determination of polypeptide variants according to the present invention. The use of the BLAST family of algorithms, including BLASTN, BLASTP and BLASTX, is described in the publication of Altschul et al., Nucleic Acids Res. 25: 3389-3402, 1997.

The computer algorithm FASTA is available on the Internet and from the University of Virginia by contacting David Hudson, Vice Provost for Research, University of Virginia, P.O. Box 9025, Charlottesville, Va. 22906-9025, USA. FASTA Version 2.0u4 [February 1996], set to the default parameters described in the documentation and distributed with the algorithm, may be used in the determination of variants according to the present invention. The use of the FASTA algorithm is described in Pearson and Lipman, Proc. Natl. Acad. Sci. USA 85:2444-2448, 1988; and Pearson, Methods in Enzymol. 183: 63-98, 1990.

The following running parameters are preferred for determination of alignments and similarities using BLASTN that contribute to the E values and percentage identity for polynucleotide sequences: Unix running command: blastall-p blastn-d embldb-e 10-G0-E0-r1-v30-b30-i queryseq-o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (BLASTN only) [Integer]; -v Number of one-line descriptions (V) [Integer]; -b Number of alignments to show (B) [Integer]; -i Query File [File In]; and -o BLAST report Output File [File Out] Optional.

The following running parameters are preferred for determination of alignments and similarities using BLASTP that contribute to the E values and percentage identity of polypeptide sequences: blastall-p blastp-d swissprottrembledb-e10-G0-E0-v30-b30-i queryseq-o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -v Number of one-line descriptions (v) [Integer]; -b Number of alignments to show (b) [Integer]; -I Query File [File In]; -o BLAST report Output File [File Out] Optional. The “hits” to one or more database sequences by a queried sequence produced by BLASTN, FASTA, BLASTP or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, FASTA, and BLASTP algorithms also produce “Expect” values for alignments. The Expect value (E) indicates the number of hits one can “expect” to see over a certain number of contiguous sequences by chance when searching a database of a certain size. The Expect value is used as a significance threshold for determining whether the hit to a database, such as the preferred EMBL database, indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the polynucleotide sequences then have a probability of 90% of being the same. For sequences having an E value of 0.01 or less over aligned and matched portions, the probability of finding a match by chance in the EMBL database is 1% or less using the BLASTN or FASTA algorithm.

According to one embodiment, “variant” polynucleotides and polypeptides, with reference to each of the polynucleotides and polypeptides of the present invention, preferably comprise sequences producing an E value of 0.01 or less when compared to the polynucleotide or polypeptide of the present invention. That is, a variant polynucleotide or polypeptide is any sequence that has at least a 99% probability of being the same as the polynucleotide or polypeptide of the present invention, measured as having an E value of 0.01 or less using the BLASTN, FASTA, or BLASTP algorithms set at parameters described above. According to a preferred embodiment, a variant polynucleotide is a sequence having the same number or fewer nucleic acids than a polynucleotide of the present invention that has at least a 99% probability of being the same as the polynucleotide of the present invention, measured as having an E value of 0.01 or less using the BLASTN or FASTA algorithms set at parameters described above. Similarly, according to a preferred embodiment, a variant polypeptide is a sequence having the same number or fewer amino acids than a polypeptide of the present invention that has at least a 99% probability of being the same as a polypeptide of the present invention, measured as having an E value of 0.01 or less using the BLASTP algorithm set at the parameters described above.

As noted above, the percentage identity is determined by aligning sequences using one of the BLASTN, FASTA, or BLASTP algorithms, set at the running parameters described above, and identifying the number of identical nucleic or amino acids over the aligned portions; dividing the number of identical nucleic or amino acids by the total number of nucleic or amino acids of the polynucleotide or polypeptide sequence of the present invention; and then multiplying by 100 to determine the percentage identity. For example, a polynucleotide of the present invention having 220 nucleic acids has a hit to a polynucleotide sequence in the EMBL database having 520 nucleic acids over a stretch of 23 nucleotides in the alignment produced by the BLASTN algorithm using the parameters described above. The 23 nucleotide hit includes 21 identical nucleotides, one gap and one different nucleotide. The percentage identity of the polynucleotide of the present invention to the hit in the EMBL library is thus 21/220 times 100, or 9.5%. The polynucleotide sequence in the EMBL database is thus not a variant of a polynucleotide of the present invention.

In addition to having a specified percentage identity to an inventive polynucleotide or polypeptide sequence, variant polynucleotides and polypeptides preferably have additional structure and/or functional features in common with the inventive polynucleotide or polypeptide. Polypeptides having a specified degree of identity to a polypeptide of the present invention share a high degree of similarity in their primary structure and have substantially similar functional properties. In addition to sharing a high degree of similarity in their primary structure to polynucleotides of the present invention, polynucleotides having a specified degree of identity to, or capable of hybridizing to an inventive polynucleotide preferably have at least one of the following features: (i) they contain an open reading frame or partial open reading frame encoding a polypeptide having substantially the same functional properties as the polypeptide encoded by the inventive polynucleotide; or (ii) they contain identifiable domains in common.

Alternatively, variant polynucleotides of the present invention hybridize to the polynucleotide sequences recited in SEQ ID NOS: 1-80, or complements, reverse sequences, or reverse complements of those sequences under stringent conditions. As used herein, “stringent conditions” refers to prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The present invention also encompasses polynucleotides that differ from the disclosed sequences but that, as a consequence of the discrepancy of the genetic code, encode a polypeptide having similar enzymatic activity as a polypeptide encoded by a polynucleotide of the present invention. Thus, polynucleotides comprising sequences that differ from the polynucleotide sequences recited in SEQ ID NOS: 1-80, or complements, reverse sequences, or reverse complements of those sequences as a result of conservative substitutions are encompassed within the present invention. Additionally, polynucleotides comprising sequences that differ from the inventive polynucleotide sequences or complements, reverse complements, or reverse sequences as a result of deletions and/or insertions totaling less than 10% of the total sequence length are also contemplated by and encompassed within the present invention. Similarly, polypeptides comprising sequences that differ from the inventive polypeptide sequences as a result of amino acid substitutions, insertions, and/or deletions totaling less than 10% of the total sequence length are contemplated by and encompassed within the present invention, provided the variant polypeptide has similar activity to the inventive polypeptide.

The polynucleotides of the present invention may be isolated from various libraries, or may be synthesized using techniques that are well known in the art. The polynucleotides may be synthesized, for example, using automated oligonucleotide synthesizers (e.g., Beckman Oligo 1000M DNA Synthesizer) to obtain polynucleotide segments of up to 50 or more nucleic acids. A plurality of such polynucleotide segments may then be ligated using standard DNA manipulation techniques that are well known in the art of molecular biology. One conventional and exemplary polynucleotide synthesis technique involves synthesis of a single stranded polynucleotide segment having, for example, 80 nucleic acids, and hybridizing that segment to a synthesized complementary 85 nucleic acid segment to produce a 5-nucleotide overhang. The next segment may then be synthesized in a similar fashion, with a 5-nucleotide overhang on the opposite strand. The “sticky” ends ensure proper ligation when the two portions are hybridized. In this way, a complete polynucleotide of the present invention may be synthesized entirely in vitro.

Certain of the polynucleotides identified as SEQ ID NOS: 1-80 are generally referred to as “partial” sequences, in that they may not represent the full coding portion of a gene encoding a naturally occurring polypeptide. The partial polynucleotide sequences disclosed herein may be employed to obtain the corresponding full-length genes for various species and organisms by, for example, screening DNA expression libraries using hybridization probes based on the polynucleotides of the present invention, or using PCR amplification with primers based upon the polynucleotides of the present invention. In this way one can, using methods well known in the art, extend a polynucleotide of the present invention upstream and downstream of the corresponding DNA, as well as identify the corresponding mRNA and genomic DNA, including the promoter and enhancer regions, of the complete gene. The present invention thus comprehends isolated polynucleotides comprising a sequence identified in SEQ ID NOS: 1-80, or a variant of one of the specified sequences, that encode a functional polypeptide, including full length genes. Such extended polynucleotides may have a length of from about 50 to about 4,000 nucleic acids or base pairs, and preferably have a length of less than about-4,000 nucleic acids or base pairs, more preferably yet a length of less than about 3,000 nucleic acids or base pairs, more preferably yet a length of less than about 2,000 nucleic acids or base pairs. Under some circumstances, extended polynucleotides of the present invention may have a length of less than about 1,800 nucleic acids or base pairs, preferably less than about 1,600 nucleic acids or base pairs, more preferably less than about 1,400 nucleic acids or base pairs, more preferably yet less than about 1,200 nucleic acids or base pairs, and most preferably less than about 1,000 nucleic acids or base pairs.

Polynucleotides of the present invention comprehend polynucleotides comprising at least a specified number of contiguous residues α-mers) of any of the polynucleotides identified as SEQ ID NOS: 1-80 or their variants. According to preferred embodiments, the value of x is preferably at least 20, more preferably at least 40, more preferably yet at least 60, and most preferably at least 80. Thus, polynucleotides of the present invention include polynucleotides comprising a 20-mer, a 40-mer, a 60-mer, an 80-mer, a 100-mer, a 120-mer, a 150-mer, a 180-mer, a 220-mer a 250-mer, or a 300-mer, 400-mer, 500-mer or 600-mer of a polynucleotide identified as SEQ ID NOS: 1-80 or a variant of one of the polynucleotides identified as SEQ ID NOS: 1-80.

Oligonucleotide probes and primers complementary to and/or corresponding to SEQ ID NOS: 1-80, and variants of those sequences, are also comprehended by the present invention. Such oligonucleotide probes and primers are substantially complementary to the polynucleotide of interest. An oligonucleotide probe or primer is described as “corresponding to” a polynucleotide of the present invention, including one of the sequences set out as SEQ ID NOS: 1-80 or a variant, if the oligonucleotide probe or primer, or its complement, is contained within one of the sequences set out as SEQ ID. NOS: 1-80 or a variant of one of the specified sequences.

Two single stranded sequences are said to be substantially complementary when the nucleotides of one strand, optimally aligned and compared, with the appropriate nucleotide insertions and/or deletions, pair with at least 80%, preferably at least 90% to 95%, and more preferably at least 98% to 100%, of the nucleotides of the other strand. Alternatively, substantial complementarity exists when a first DNA strand will selectively hybridize to a second DNA strand under stringent hybridization conditions. Stringent hybridization conditions for determining complementarity include salt conditions of less than about 1 M, more usually less than about 500 mM and preferably less than about 200 mM. Hybridization temperatures can be as low as 5° C., but are generally greater than about 22° C., more preferably greater than about 30° C. and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone. DNA-DNA hybridization studies may performed using either genomic DNA or DNA derived by preparing cDNA from the RNA present in a sample to be tested.

In addition to DNA-DNA hybridization, DNA-RNA or RNA-RNA hybridization assays are also possible. In the first case, the mRNA from expressed genes would then be detected instead of genomic DNA or cDNA derived from mRNA of the sample. In the second case, RNA probes could be used. In addition, artificial analogs of DNA hybridizing specifically to target sequences could also be used.

In specific embodiments, the oligonucleotide probes and/or primers comprise at least about 6 contiguous residues, more preferably at least about 10 contiguous residues, and most preferably at least about 20 contiguous residues complementary to a polynucleotide sequence of the present invention. Probes and primers of the present invention may be from about 8 to 100 base pairs in length or, preferably from about 10 to 50 base pairs in length or, more preferably from about 15 to 40 base pairs in length. The primers and probes may be readily selected using procedures well known in the art, taking into account DNA-DNA hybridization stringencies, annealing and melting temperatures, potential for formation of loops and other factors, which are well known in the art. Tools and software suitable for designing probes, and especially for designing PCR primers, are available from Premier Biosoft International, 3786 Corina Way, Palo Alto, Calif. 94303-4504. Preferred techniques for designing PCR primers are also disclosed in Dieffenbach and Dyksler, PCR primer: a laboratory manual, CSHL Press: Cold Spring Harbor, N.Y., 1995.

A plurality of oligonucleotide probes or primers corresponding to a polynucleotide of the present invention may be provided in a kit form. Such kits generally comprise multiple DNA or oligonucleotide probes, each probe being specific for a polynucleotide sequence. Kits of the present invention may comprise one or more probes or primers corresponding to a polynucleotide of the present invention, including a polynucleotide sequence identified in SEQ ID NOS: 1-80.

In one embodiment useful for high-throughput assays, the oligonucleotide probe kits of the present invention comprise multiple probes in an array format, wherein each probe is immobilized in a predefined, spatially addressable location on the surface of a solid substrate. Array formats which may be usefully employed in the present invention are disclosed, for example, in U.S. Pat. Nos. 5,412,087, 5,545,531, and PCT Publication No. WO 95/00530, the disclosures of which are hereby incorporated by reference.

Oligonucleotide probes for use in the present invention may be constructed synthetically prior to immobilization on an array, using techniques well known in the art (See, for example, Gait, ed., Oligonucleotide synthesis a practical approach, IRL Press: Oxford, England, 1984). Automated equipment for the synthesis of oligonucleotides is available commercially from such companies as Perkin Elmer/Applied Biosystems Division (Foster City, Calif.) and may be operated according to the manufacturer's instructions. Alternatively, the probes may be constructed directly on the surface of the array using techniques taught, for example, in PCT Publication No. WO 95/00530.

The solid substrate and the surface thereof preferably form a rigid support and are generally formed from the same material. Examples of materials from which the solid substrate may be constructed include polymers, plastics, resins, membranes, polysaccharides, silica or silica-based materials, carbon, metals and inorganic glasses. Synthetically prepared probes may be immobilized on the surface of the solid substrate using techniques well known in the art, such as those disclosed in U.S. Pat. No. 5,412,087.

In one such technique, compounds having protected functional groups, such as thiols protected with photochemically removable protecting groups, are attached to the surface of the substrate. Selected regions of the surface are then irradiated with a light source, preferably a laser, to provide reactive thiol groups. This irradiation step is generally performed using a mask having apertures at predefined locations using photolithographic techniques well known in the art of semiconductors. The reactive thiol groups are then incubated with the oligonucleotide probe to be immobilized. The precise conditions for incubation, such as temperature, time and pH, depend on the specific probe and can be easily determined by one of skill in the art. The surface of the substrate is washed free of unbound probe and the irradiation step is repeated using a second mask having a different pattern of apertures. The surface is subsequently incubated with a second, different, probe. Each oligonucleotide probe is typically immobilized in a discrete area of less than about 1 mm². Preferably each discrete area is less than about 10,000 mm², more preferably less than about 100 mm². In this manner, a multitude of oligonucleotide probes may be immobilized at predefined locations on the array.

The resulting array may be employed to screen for differences in organisms or samples or products containing genetic material as follows. Genomic or cDNA libraries are prepared using techniques well known in the art. The resulting target DNA is then labeled with a suitable marker, such as a radiolabel, chromophore, fluorophore or chemiluminescent agent, using protocols well known for those skilled in the art. A solution of the labeled target DNA is contacted with the surface of the array and incubated for a suitable period of time.

The surface of the array is then washed free of unbound target DNA and the probes to which the target DNA hybridized are determined by identifying those regions of the array to which the markers are attached. When the marker is a radiolabel, such as ³²P, autoradiography is employed as the detection method. In one embodiment, the marker is a fluorophore, such as fluorescein, and the location of bound target DNA is determined by means of fluorescence spectroscopy. Automated equipment for use in fluorescence scanning of oligonucleotide probe arrays is available from Affymetrix, Inc. (Santa Clara, Calif.) and may be operated according to the manufacturer's instructions. Such equipment may be employed to determine the intensity of fluorescence at each predefined location on the array, thereby providing a measure of the amount of target DNA bound at each location. Such an assay would be able to indicate not only the absence and presence of the marker probe in the target, but also the quantitative amount as well.

The significance of such high-throughput screening system is apparent for applications such as microbial selection and quality control operations in which there is a need to identify large numbers of samples or products for unwanted materials, to identify microbes or samples or products containing microbial material for quarantine purposes, etc., or to ascertain the true origin of samples or products containing microbes. Screening for the presence or absence of polynucleotides of the present invention used as identifiers for tagging microbes and microbial products can be valuable for later detecting the genetic composition of food, fermentation and industrial microbes or microbes in human or animal digestive system after consumption of probiotics, etc.

In this manner, oligonucleotide probe kits of the present invention may be employed to examine the presence/absence (or relative amounts in case of mixtures) of polynucleotides in different samples or products containing different materials rapidly and in a cost-effective manner. Examples of microbial species which may be examined using the present invention, include lactic acid bacteria, such as Lactobacillus rhamnosus, and other microbial species.

Another aspect of the present invention involves collections of a plurality of polynucleotides of the present invention. A collection of a plurality of the polynucleotides of the present invention, particularly the polynucleotides identified as SEQ ID NOS: 1-80, may be recorded and/or stored on a storage medium and subsequently accessed for purposes of analysis, comparison, etc. Suitable storage media include magnetic media such as magnetic diskettes, magnetic tapes, CD-ROM storage media, optical storage media, and the like. Suitable storage media and methods for recording and storing information, as well as accessing information such as polynucleotide sequences recorded on such media, are well known in the art. The polynucleotide information stored on the storage medium is preferably computer-readable and may be used for analysis and comparison of the polynucleotide information.

Another aspect of the present invention thus involves storage medium on which are recorded a collection of the polynucleotides of the present invention, particularly a collection of the polynucleotides identified as SEQ ID NOS: 1-80. According to one embodiment, the storage medium includes a collection of at least 20, preferably at least 50, more preferably at least 100, and most preferably at least 200 of the polynucleotides of the present invention, preferably the polynucleotides identified as SEQ ID NOS: 1-80, including variants of those polynucleotides.

Another aspect of the present invention involves a combination of polynucleotides, the combination containing at least 5, preferably at least 10, more preferably at least 20, and most preferably at least 50 different polynucleotides of the present invention, including polynucleotides selected from SEQ ID NOS: 1-80, and variants of these polynucleotides.

In another aspect, the present invention provides genetic constructs comprising, in the 5′-3′ direction, a gene promoter sequence and an open reading frame coding for at least a functional portion of a polypeptide encoded by a polynucleotide of the present invention. In certain embodiments, the genetic constructs of the present invention also comprise a gene termination sequence. The open reading frame may be oriented in either a sense or antisense direction. Genetic constructs comprising a non-coding region of a gene coding for a polypeptide encoded by an inventive polynucleotide or a nucleotide sequence complementary to a non-coding region, together with a gene promoter sequence, are also provided. A terminator sequence may form part of this construct. Preferably, the gene promoter and termination sequences are functional in a host organism. More preferably, the gene promoter and termination sequences are common to those of the polynucleotide being introduced. The genetic construct may further include a marker for the identification of transformed cells.

Techniques for operatively linking the components of the genetic constructs are well known in the art and include the use of synthetic linkers containing one or more restriction endonuclease sites as described, for example, by Sambrook et al., in Molecular cloning: a laboratory manual, Cold Spring Harbor Laboratories Press: Cold Spring Harbor, N.Y., 1989. The genetic constructs of the present invention may be linked to a vector having at least one replication system, for example, E. coli, whereby after each manipulation, the resulting construct can be cloned and sequenced and the correctness of the manipulation determined.

Transgenic microbial cells comprising the genetic constructs of the present invention are also provided by the present invention, together with microbes comprising such transgenic cells, products and progeny of such microbes, and materials including such microbes. Techniques for stably incorporating genetic constructs into the genome of target microbes, such as Lactobacillus species, Lactococcus lactis or E. coli, are well known in the art of bacterial transformation and are exemplified by the transformation of E. coli for sequencing described in Example 1.

Transgenic non-microbial cells comprising the genetic constructs of the present invention are also provided, together with organisms comprising such transgenic cells, and products and progeny of such organisms. Genetic constructs of the present invention may be stably incorporated into the genomes of non-microbial target organisms, such as fungi, using techniques well known in the art.

In preferred embodiments, the genetic constructs of the present invention are employed to transform microbes used in the production of food products, ingredients, processing aids, additives or supplements and for the production of microbial products for pharmaceutical uses, particularly for modulating immune system function and immunological effects, and in the production of chemoprotectants providing beneficial effects, probiotics and health supplements. The inventive genetic constructs may also be employed to transform bacteria that are used to produce enzymes or substances such as polysaccharides, flavor compounds and bioactive substances, and to enhance resistance to industrial processes such as drying and to adverse stimuli in the human digestive system. The genes involved in antibiotic production, and phage uptake and resistance in Lactobacillus rhamnosus are considered to be especially useful. The target microbe to be used for transformation with one or more polynucleotides or genetic constructs of the present invention is preferably selected from the group consisting of bacterial genera Lactococcus, Lactobacillus, Streptococcus, Oenococcus, Lactosphaera, Trichococcus, Pediococcus and others potentially useful in various fermentation industries and is most preferably selected from the group consisting of the following Lactobacillus species: Lactobacillus acetotolerans, Lactobacillus acidophilus, Lactobacillus agilis, Lactobacillus alimentarius, Lactobacillus amylolyticus, Lactobacillus amylophilus, Lactobacillus amylovorus, Lactobacillus animalis, Lactobacillus arizonae, Lactobacillus aviarius, Lactobacillus bavaricus, Lactobacillus bifermentans, Lactobacillus brevis, Lactobacillus buchneri, Lactobacillus bulgaricus, Lactobacillus casei, Lactobacillus collinoides, Lactobacillus coryniformis, Lactobacillus crispatus, Lactobacillus curvatus, Lactobacillus delbrueckii, Lactobacillus delbrueckii subsp. bulgaricus, Lactobacillus delbrueckii subsp. lactis, Lactobacillus farciminis, Lactobacillus fermentum, Lactobacillus fructivorans, Lactobacillus gallinarum, Lactobacillus gasseri, Lactobacillus graminis, Lactobacillus hamsteri, Lactobacillus helveticus, Lactobacillus helveticus subsp. jugurti, Lactobacillus hetero, Lactobacillus hilgardii, Lactobacillus homohiochii, Lactobacillus japonicus, Lactobacillus johnsonii, Lactobacillus kefiri, Lactobacillus lactis, Lactobacillus leichmannii, Lactobacillus lindneri, Lactobacillus mali, Lactobacillus maltaromicus, Lactobacillus manihotivorans, Lactobacillus mucosae, Lactobacillus murinus, Lactobacillus oris, Lactobacillus panis, Lactobacillus paracasei, Lactobacillus paracasei subsp. pseudoplantarum, Lactobacillus paraplantarum, Lactobacillus pentosus, Lactobacillus plantarum, Lactobacillus pontis, Lactobacillus reuteri, Lactobacillus rhamnosus, Lactobacillus ruminis, Lactobacillus sake, Lactobacillus salivarius, Lactobacillus salivarius subsp. salicinius, Lactobacillus salivarius subsp. salivarius, Lactobacillus sanfranciscensis, Lactobacillus sharpeae, Lactobacillus thermophilus, Lactobacillus vaginalis, Lactobacillus vermiforme, and Lactobacillus zeae.

In yet a further aspect, the present invention provides methods for modifying the concentration, composition and/or activity of a polypeptide in a host organism, such as a microbe, comprising stably incorporating a genetic construct of the present invention into the genome of the host organism by transforming the host organism with such a genetic construct. The genetic constructs of the present invention may be used to transform a variety of organisms including plants, such as monocotyledonous angiosperms (e.g., grasses, corn, grains, oat, wheat and barley); dicotyledonous angiosperms (e.g., Arabidopsis, tobacco, legumes, alfalfa, oaks, eucalyptus, maple); gymnosperms, (e.g., Scots pine (Aronen, Finnish Forest Res. Papers, Vol. 595, 1996); white spruce (Ellis et al., Biotechnology 11:84-89, 1993); larch (Huang, et al., In Vitro Cell 27:201-207, 1991); and any kind of plant amenable to genetic engineering.

Thus, in yet another aspect, transgenic plant cells comprising the genetic constructs of the present invention are provided, together with plants comprising such transgenic cells, and fruits, seeds, products and progeny of such plants. Techniques for stably incorporating genetic constructs into the genome of target organisms, such as plants, are well known in the art and include Agrobacterium tumefaciens mediated introduction, electroporation, protoplast fusion, injection into reproductive organs, injection into immature embryos, high velocity projectile introduction and the like. The choice of technique will depend upon the target plant to be transformed. For example, dicotyledonous plants, and certain monocots and gymnosperms, may be transformed by Agrobacterium Ti plasmid technology, as described, for example by Bevan, Nucleic Acids Res. 12:8711-8721, 1984. Targets for the introduction of the genetic constructs include tissues, such as leaf tissue, disseminated cells, protoplasts, seeds, embryos, meristematic regions, cotyledons, hypocotyls, and the like.

Once the cells are transformed, cells having the genetic construct incorporated in their genome are selected. Transgenic cells may then be cultured in an appropriate medium, using techniques well known in the art. In the case of protoplasts, the cell wall is allowed to reform under appropriate osmotic conditions. In the case of seeds or embryos, an appropriate germination or callus initiation medium is employed. For explants, an appropriate regeneration medium is used. Regeneration of plants is well established for many species. For a review of regeneration of forest trees, see Dunstan et al., “Somatic embryogenesis in woody plants,” in Thorpe, T. A., ed., In vitro embryogenesis of plants, (Current Plant Science and Biotechnology in Agriculture), 20(12):471-540, 1995. Specific protocols for the regeneration of spruce are discussed by Roberts et al. (“Somatic embryogenesis of Spruce,” in Redenbaugh K., ed., Synseed: applications of synthetic seed to crop improvement, CRC Press: Ch.23:427-449, 1993). The resulting transformed plants may be reproduced sexually or asexually, using methods well known in the art, to give successive generations of transgenic plants and practically unlimited amounts of tagged plant-derived products.

The polynucleotides of the present invention may be further employed as non-disruptive tags for marking organisms, particularly microbes. Other organisms may, however, be tagged with the polynucleotides of the present invention, including commercially valuable plants, animals, fish, fungi and yeasts. Genetic constructs comprising polynucleotides of the present invention may be stably introduced into an organism as heterologous, non-functional, non-disruptive tags. It is then possible to identify the origin or source of the organism at a later date by determining the presence or absence of the tag(s) in a sample of material. Detection of the tag(s) may be accomplished using a variety of conventional techniques, and will generally involve the use of nucleic acid probes. Sensitivity in assaying the presence of probe can be usefully increased by using branched oligonucleotides, as described by Horn et al., Nucleic Acids Res. 25(23):4842-4849, 1997, enabling detection of as few as 50 DNA molecules in the sample.

Polynucleotides of the present invention may also be used to specifically suppress gene expression by methods that operate post-transcriptionally to block the synthesis of products of targeted genes, such as RNA interference (RNAi), and quelling. Briefly, traditional methods of gene suppression, employing anti-sense RNA or DNA, operate by binding to the reverse sequence of a gene of interest such that binding interferes with subsequent cellular processes and therefore blocks synthesis of the corresponding protein. RNAi also operates on a post-translational level and is sequence specific, but suppresses gene expression far more efficiently. Exemplary methods for controlling or modifying gene expression using RNAi are provided in WO 99/49029 and WO 99/53050. In these methods, post-transcriptional gene silencing is brought about by a sequence-specific RNA degradation process which results in the rapid degradation of transcripts of sequence-related genes. Studies have shown that double-stranded RNA may act as a mediator of sequence-specific gene silencing (see, for example, Montgomery and Fire, Trends in Genetics, 14:255-258, 1998). Gene constructs that produce transcripts with self-complementary regions are particularly efficient at gene silencing. A unique feature of this post-transcriptional gene silencing pathway is that silencing is not limited to the cells where it is initiated. The gene-silencing effects may be disseminated to other parts of an organism and even transmitted through the germ line to several generations.

The polynucleotides of the present invention may thus be employed to generate gene silencing constructs and/or gene-specific self-complementary RNA sequences that can be delivered by conventional art-known methods to cells, such as microbial cells. Within genetic constructs, sense and antisense sequences can be placed in regions flanking an intron sequence in proper splicing orientation with donor and acceptor splicing sites, such that intron sequences are removed during processing of the transcript and sense and antisense sequences, as well as splice junction sequences, bind together to form double-stranded RNA. Alternatively, spacer sequences of various lengths may be employed to separate self-complementary regions of sequence in the construct. During processing of the gene construct transcript, intron sequences are spliced-out, allowing sense and anti-sense sequences, as well as splice junction sequences, to bind forming double-stranded RNA. Select ribonucleases then bind to and cleave the double-stranded RNA, thereby initiating the cascade of events leading to degradation of specific mRNA gene sequences, and silencing specific genes. Alternatively, rather than using a gene construct to express the self-complementary RNA sequences, the gene-specific double-stranded RNA segments are delivered to one or more targeted areas to be internalized into the cell cytoplasm to exert a gene silencing effect. The double-stranded RNA must have sufficient homology to the targeted gene to mediate RNAi and is preferably at least 25 nucleotides in length. Preferably, the double-stranded RNA corresponds specifically to a polynucleotide of the present invention. Gene silencing RNA sequences comprising the polynucleotides of the present invention are useful for creating genetically modified organisms, such as microbes, with desired phenotypes as well as for characterizing genes (for example, in high-throughput screening of sequences), and studying their functions in intact organisms.

In another aspect, the present invention provides methods for using one or more of the inventive polypeptides or polynucleotides to treat disorders in a mammal, such as a human.

In this aspect, the polypeptide or polynucleotide is generally present within a composition, such as a pharmaceutical or immunogenic composition. Pharmaceutical compositions may comprise one or more polypeptides, each of which may contain one or more of the above sequences (or variants thereof), and a physiologically acceptable carrier. Immunogenic compositions may comprise one or more of the above polypeptides and an immunostimulant, such as an adjuvant or a liposome, into which the polypeptide is incorporated.

Alternatively, a composition of the present invention may contain DNA encoding one or more polypeptides described herein, such that the polypeptide is generated in situ. In such compositions, the DNA may be present within any of a variety of delivery systems known to those of ordinary skill in the art, including nucleic acid expression systems, and bacterial and viral expression systems. Appropriate nucleic acid expression systems contain the necessary DNA sequences for expression in the patient (such as a suitable promoter and terminator signal). Bacterial delivery systems involve the administration of a bacterium (such as Bacillus Calmette-Guerin) that expresses an immunogenic portion of the polypeptide on its cell surface. In a preferred embodiment, the DNA may be introduced using a viral expression system (e.g., vaccinia or other poxvirus, retrovirus, or adenovirus), which may involve the use of a non-pathogenic, or defective, replication competent virus. Techniques for incorporating DNA into such expression systems are well known in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. For parenteral administration, such as subcutaneous injection, the carrier preferably comprises water, saline, alcohol, a lipid, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the immunogenic compositions of the present invention to non-specifically enhance an immune response. Most adjuvants contain a substance designed to protect the antigen from rapid catabolism, such as aluminum hydroxide or mineral oil, and a non-specific stimulator of immune responses, such as lipid A, Bordetella pertussis or M. tuberculosis. Suitable adjuvants are commercially available as, for example, Freund's Incomplete Adjuvant and Freund's Complete Adjuvant (Difco Laboratories, Detroit, Mich.), and Merck Adjuvant 65 (Merck and Company, Inc., Rahway, N.J.). Other suitable adjuvants include alum, biodegradable microspheres, monophosphoryl lipid A and Quil A.

Routes and frequency of administration, as well as dosage, vary from individual to individual. In general, the inventive compositions may be administered by injection (e.g., intradermal, intramuscular, intravenous or subcutaneous), intranasally (e.g., by aspiration) or orally. In general, the amount of polypeptide present in a dose (or produced in situ by the DNA in a dose) ranges from about 1 pg to about 100 mg per kg of host, typically from about 10 pg to about 1 mg per kg of host, and preferably from about 100 pg to about 1 μg per kg of host. Suitable dose sizes will vary with the size of the patient, but will typically range from about 0.1 ml to about 2 ml.

The following examples are offered by way of illustration and not by way of limitation.

### EXAMPLE 1

### Isolation and Characterization of DNA Sequences from Lactobacillus Rhamnosus Strain HN001

Lactobacillus rhamnosus strain HN001 DNA libraries were constructed and screened as follows.

DNA was prepared in large scale by cultivating the bacteria in 2×100 ml cultures with 100 ml MRS broth (Difco Laboratories, Detroit Mich.) and 1 ml Lactobacillus glycerol stock as inoculum, placed into 500 ml culture flasks and incubated at 37° C. for approx. 16 hours with shaking (220 rpm).

The cultures were centrifuged at 3500 rpm for 10 min to pellet the cells. The supernatant was removed and the cell pellet resuspended in 40 ml fresh MRS broth and transferred to clean 500 ml culture flasks. Fresh MRS broth (60 ml) was added to bring the volume back to 100 ml and flasks were incubated for a further 2 hrs at 37° C. with shaking (220 rpm). The cells were pelleted by centrifugation (3500 rpm for 10 min) and supernatant removed. Cell pellets were washed twice in 20 ml buffer A (50 mM NaCl, 30 mM Tris pH 8.0, 0.5 mM EDTA).

Cells were resuspended in 2.5 ml buffer B (25% sucrose (w/v), 50 mM Tris pH 8.0, 1 mM EDTA, 20 mg/ml lysozyme, 20 μg/ml mutanolysin) and incubated at 37° C. for 45 min. Equal volumes of EDTA (0.25 M) was added to each tube and allowed to incubate at room temperature for 5 min. 20% SDS (1 ml) solution was added, mixed and incubated at 65° C. for 90 min. 50 μl Proteinase K (Gibco BRL, Gaithersburg, Md.) from a stock solution of 20 mg/ml was added and tubes incubated at 65° C. for 15 min.

DNA was extracted with equal volumes of phenol:chloroform:isoamylalcohol (25:24:1). Tubes were centrifuged at 3500 rpm for 40 min. The aqueous phase was removed to clean sterile Oak Ridge centrifuge tubes (30 ml). Crude DNA was precipitated with an equal volume of cold isopropanol and incubated at −20° C. overnight.

After resuspension in 500 μl TE buffer, DNase-free RNase was added to a final concentraion of 100 μg/ml and incubated at 37° C. for 30 min. The incubation was extended for a further 30 min after adding 100 μl Proteinase K from a stock solution of 20 mg/ml. DNA was precipitated with ethanol after a phenol:chloroform:isoamylalcohol (25:24:1) and a chloroform:isoamylalcohol (24:1) extraction and dissolved in 250 μl TE buffer.

DNA was digested with Sau3AI at a concentration of 0.004 U/μg in a total volume of 1480 μl, with 996 μl DNA, 138.75 μl 10× REACT 4 buffer and 252.75 μl H₂O. Following incubation for 1 hour at 37° C., DNA was divided into two tubes. 31 μl 0.5 M EDTA was added to stop the digestion and 17 μl samples were taken for agarose gel analysis. Samples were put into 15 ml Falcon tubes and diluted to 3 ml for loading onto sucrose gradient tubes.

Sucrose gradient size fractionation was conducted as follows. 100 ml of 50% sucrose (w/v) was made in TEN buffer (1M NaCl, 20 mM Tris pH 8.0, 5 mM EDTA) and sterile filtered. Dilutions of 5, 10, 15, 20, 25, 30, 35 and 40% sucrose were prepared and overlaid carefully in Beckman Polyallomer tubes, and kept overnight at 4° C. TEN buffer (4 ml) was loaded onto the gradient, with 3 ml of DNA solution on top. The gradients were centrifuged at 26K for 18 hours at 4° C. in a Centricon T-2060 centrifuge using a Kontron TST 28-38 rotor. After deceleration without braking (approx. 1 hour), the gradients were removed and fractions collected using an auto Densi-Flow (Haake-Buchler Instruments). Agarose gel was used to analyze the fractions. The best two pairs of fractions were pooled and diluted to contain less than 10% sucrose. TEN buffer (4 ml) was added and DNA precipitated with 2 volumes of 100% ice cold ethanol and an overnight incubation at −20° C.

DNA pellets were resuspended in 300 μl TE buffer and re-precipitated for approx. 6 hours at −20° C. after adding 1/10 volume 3 M NaOAC pH 5.2 and 2 volumes of ethanol. DNA was pelleted at top speed in a microcentrifuge for 15 min, washed with 70% ethanol and pelleted again, dried and resuspended in 10 μl TE buffer.

DNA was ligated into dephosphorylated BamHI-digested pBluescript SK II⁺ and dephosphorylated BamHI-digested lambda ZAP Express using standard protocols. Packaging of the DNA was done using Gigapack III Gold packaging extract (Stratagene, La Jolla, Calif.) following the manufacturer's protocols. Packaged libraries were stored at 4° C.

Mass excision from the primary packaged phage library was done using XL1-Blue MRF′ cells and ExAssist Helper Phage (Stratagene). The excised phagemids were diluted with NZY broth (Gibco BRL, Gaithersburg, Md.) and plated out onto LB-kanamycin agar plates containing 5-bromo-4-chloro-3-indolyl-β-D-galactoside (X-gal) and isopropylthio-beta-galactoside (IPTG). After incubation, single colonies were picked for PCR size determination before the most suitable libraries were selected for sequencing.

Of the colonies picked for DNA minipreps and subsequent sequencing, the large majority contained an insert suitable for sequencing. Positive colonies were cultured in LB broth with kanamycin or ampicillin depending on the vector used, and DNA was purified by means of rapid alkaline lysis minipreps (solutions: Qiagen, Venlo, The Netherlands; clearing plates, Millipore, Bedford, Mass.). Agarose gels at 1% were used to screen sequencing templates for chromosomal contamination and concentration. Dye terminator sequencing reactions were prepared using a Biomek 2000 robot (Beckman Coulter, Inc., Fullerton, Calif.) and Hydra 96 (Robbins Scientific, Sunnyvale, Calif.) for liquid handling. DNA amplification was done in a 9700 PCR machine (Perkin Elmer/Applied Biosystems, Foster City, Calif.) according to the manufacturer's protocol.

The sequence of the genomic DNA fragments was determined using a Perkin Elmer/Applied Biosystems Division Prism 377 sequencer. The DNA clones were sequenced from the 5′ and/or 3′ end, and are identified as SEQ ID NOS: 1-80 disclosed herein.

This example not only shows how the sequences were obtained, but also that a bacterium (E. coli) can be stably transformed with any desired DNA fragment of the present invention for permanent marking for stable inheritance.

BLASTN Polynucleotide Analysis

The determined DNA sequences were compared to and aligned with known sequences in the public databases. Specifically, the polynucleotides identified in SEQ ID NO: 1-80 were compared to polynucleotides in the EMBL database as of Aug. 12, 2002, using BLASTN algorithm Version 2.0.11 [Jan. 20, 2000], set to the following running parameters: Unix running command: blastall-p blastn-d embldb-e 10-G0-E0-r1-v30-b30-i queryseq-o results. Multiple alignments of redundant sequences were used to build up reliable consensus sequences.

The cDNA sequences of SEQ ID NOS: 1-32 and 34-80 were determined to have less than 60% identity, determined as described above, to sequences in the EMBL database using the computer algorithm BLASTN, as described above. The cDNA sequence of SEQ ID NO: 33 was determined to have less than 90% identity, determined as described above, to sequences in the EMBL database using BLASTN, as described above.

BLASTP Amino Acid Analysis

The polypeptide sequences were compared to sequences in the SwissProt-TrEMBLE protein databases using the computer algorithm BLASTP. Comparisons of amino acid sequences provided in SEQ ID NOS: 81-183 to sequences in the SwissProt-TrEMBLE protein databases (using BLASTP) were made as of Aug. 12, 2002 using BLASTN algorithm Version 2.0.11 [Jan. 20, 2000], and the following Unix running command: blastall-p blastp-d swissprottrembledb-e10-G0-E0-v30-b30-i queryseq-o.

The predicted amino acid sequences of SEQ ID NOS: 84-86, 89, 90, 92, 95, 96, 101-103, 108, 111, 114, 116, 119-122, 124, 125, 130, 134-136, 140, 146, 147, 152, 156, 159, 162, 164, 166, 168, 175 and 183 were determined to have less than 50% identity, determined as described above, to sequences in the SWISSPROT-TrEMBLE database using the BLASTP computer algorithm as described above. The predicted amino acid sequences of SEQ ID NOS: 81-83, 88, 91, 93, 94, 97-100, 104-107, 109, 110, 112, 113, 115, 123, 127-129, 131-133, 137, 138, 141-145, 148-151, 153-155, 157, 158, 160, 161, 163, 165, 167, 169-173 and 180-182 were determined to have less than 75% identity, determined as described above, to sequences in the SWISSPROT-TrEMBLE database using the computer algorithm BLASTP, as described above. The predicted amino acid sequences of SEQ ID NOS: 87, 139 and 176-179 were determined to have less than 90% identity, determined as described above, to sequences in the SWISSPROT-TrEMBLE database using the computer algorithm BLASTP, as described above. The predicted amino acid sequences of SEQ ID NOS: 117, 118 and 126 were determined to have less than 98% identity, determined as described above, to sequences in the SWISSPROT-TrEMBLE database using the computer algorithm BLASTP, as described above.

BLASTX Polynucleotide Analysis

The isolated cDNA sequences were compared to sequences in the SwissProt-TrEMBLE protein databases using the computer algorithm BLASTX. Comparisons of DNA sequences provided in SEQ ID NOS: 1-80, to sequences in the SwissProt-TrEMBLE database (using BLASTX) were made as of Aug. 12, 2002 using BLAST algorithm Version 2.0.11 [Jan. 20, 2000], and the following Unix running command: blastall-p blastx-d swissprottrembleldb-e 10-G0-E0-v30-b30-i queryseq-o.

The cDNA sequences of SEQ ID NOS: 1-14, 16-49, 52-58, 60-72, 74-78 and 80 were determined to have less than 50% identity, determined as described above, to sequences in the SWISSPROT-TrEMBLE database using the computer algorithm BLASTX, as described above. The cDNA sequences of SEQ ID NOS: 15, 50, 51, 59, 73 and 79 were determined to have less than 75% identity, determined as described above, to sequences in the SWISSPROT-TrEMBLE database using BLASTX, as described above.

Based on similarity to known sequences, the isolated polynucleotides of the present invention identified as SEQ ID NOS: 1-80 were putatively identified as encoding polypeptides having similarity to the polypeptides shown above in Table 1. The amino acid sequences encoded by the DNA sequences of SEQ ID NO: 1-80 are provided in SEQ ID NO: 81-183, respectively.

Several of the sequences provided in SEQ ID NO: 1-80 were found to be full-length and to contain open reading frames (ORFs). These full-length sequences, the location of ORFs (by nucleotide position) contained within these sequences, and the corresponding amino acid sequences are provided in Table 2 below.

TABLE 2

| Polynucleotide | | Polypeptide |
|---|---|---|
| SEQ ID NO: | ORF | SEQ ID NO: |
| 1 | 5831-7288 | 81 |
| 2 | 4395-5630 | 82 |
| 3 | 1445-2791 | 83 |
| 4 | 316-1413 | 84 |
| 5 | 1392-2444 | 85 |
| 6 | 1-1083 | 86 |
| 7 | 2881-4071 | 87 |
| 8 | 1859-3295 | 88 |
| 9 | 265-1023 | 89 |
| 10 | 1160-2005 | 90 |
| 11 | 2324-3604 | 91 |
| 11 | 548-1696 | 92 |
| 12 | 1102-2358 | 93 |
| 12 | 188-1020 | 94 |
| 13 | 140-1138 | 95 |
| 14 | 5612-6413 | 96 |
| 15 | 84-2276 | 97 |
| 16 | 1130-2275 | 98 |
| 17 | 1644-2645 | 99 |
| 18 | 500-2404 | 100 |
| 19 | 110-1153 | 101 |
| 20 | 167-718 | 102 |
| 21 | 1-3669 | 103 |
| 22 | 2327-4951 | 104 |
| 23 | 522-1694 | 105 |
| 24 | 973-2928 | 106 |
| 25 | 133-1296 | 107 |
| 26 | 1938-3497 | 108 |
| 27 | 69-716 | 109 |
| 28 | 125-1054 | 110 |
| 29 | 84-3377 | 111 |
| 30 | 555-887 | 112 |
| 30 | 226-558 | 113 |
| 31 | 77-862 | 114 |
| 32 | 3135-4673 | 115 |
| 32 | 6384-7877 | 116 |
| 33 | 412-828 | 117 |
| 33 | 863-1663 | 118 |
| 34 | 1642-2682 | 119 |
| 35 | 814-2037 | 120 |
| 35 | 3510-5084 | 121 |
| 36 | 101-1222 | 122 |
| 37 | 113-760 | 123 |
| 38 | 1-477 | 124 |
| 39 | 12447-13400 | 125 |
| 40 | 1186-2439 | 126 |
| 41 | 7973-8812 | 127 |
| 41 | 6950-7924 | 128 |
| 41 | 1925-2773 | 129 |
| 41 | 3916-4956 | 130 |
| 41 | 1023-1895 | 131 |
| 41 | 8822-10489 | 132 |
| 42 | 102-860 | 133 |
| 43 | 3759-4343 | 134 |
| 44 | 622-1113 | 135 |
| 45 | 1129-1722 | 136 |
| 46 | 1216-2439 | 137 |
| 46 | 2345-3835 | 138 |
| 47 | 6155-8361 | 139 |
| 48 | 550-1176 | 140 |
| 49 | 455-901 | 141 |
| 51 | 31-1179 | 143 |
| 52 | 766-2142 | 144 |
| 53 | 2336-3817 | 145 |
| 54 | 7567-9057 | 146 |
| 55 | 3713-4447 | 147 |
| 56 | 426-1625 | 148 |
| 57 | 138-851 | 149 |
| 58 | 3066-4769 | 150 |
| 59 | 31-1188 | 151 |
| 60 | 132-1328 | 152 |
| 61 | 94-534 | 153 |
| 62 | 652-1998 | 154 |
| 63 | 1033-1905 | 155 |
| 63 | 1902-2789 | 156 |
| 64 | 951-1646 | 157 |
| 64 | 1824-3227 | 158 |
| 65 | 179-1030 | 159 |
| 66 | 1244-2425 | 160 |
| 67 | 6885-7589 | 161 |
| 68 | 333-1121 | 162 |
| 69 | 2638-4251 | 163 |
| 69 | 1312-2622 | 164 |
| 70 | 1587-2441 | 165 |
| 71 | 4642-6489 | 166 |
| 71 | 7554-8516 | 167 |
| 71 | 6625-7536 | 168 |
| 71 | 8518-9469 | 169 |
| 72 | 2344-3732 | 170 |
| 72 | 3755-5674 | 171 |
| 73 | 102-1874 | 172 |
| 74 | 2993-4429 | 173 |
| 75 | 1008-1499 | 174 |
| 76 | 3565-5967 | 175 |
| 77 | 759-1708 | 176 |
| 77 | 1488-2837 | 177 |
| 77 | 3295-4074 | 178 |
| 77 | 4071-5096 | 179 |
| 77 | 2692-3291 | 180 |
| 78 | 165-842 | 181 |
| 79 | 66-2291 | 182 |
| 80 | 269-1006 | 183 |

### EXAMPLE 2

### Isolation and Characterization of Purine Nucleoside Phosphorylase from L. Rhamnosus Strain HN001

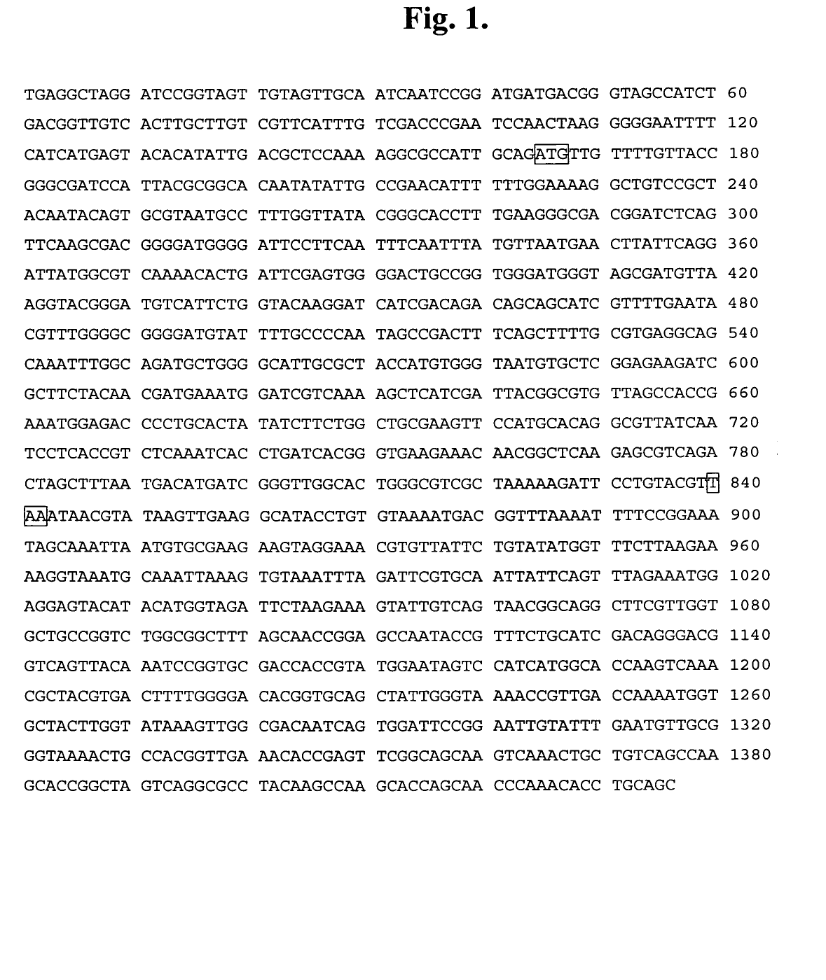

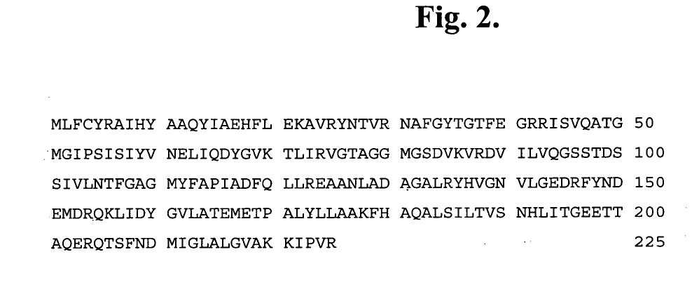

The full-length polynucleotide sequence of the deoD purine nucleoside phosphorylase gene AQ1 from L. rhamnosus HN001 is given in SEQ ID NO: 78 and shown in FIG. 1 (with ATG initiation and translation stop codons boxed). The polypeptide sequence of AQ1 is given in SEQ ID NO: 181 and shown in FIG. 2.

A 634 bp internal AQ1 fragment was amplified by PCR using standard laboratory protocols. The nucleotide sequences of the oligonucleotide primers are given in SEQ ID NOS: 184 and 185. The fragments were cloned into the pBEry1 vector cut with SmaI. The 3.6 kb pBEry1 vector was constructed using the replicon and multiple cloning site (MCS) from the phagemid pBlueScript (pBS-SK+) (Stratagene, La Jolla Calif., USA). The ampicillin resistance gene in pBS-SK+ was removed by digestion with RcaI (Roche, Auckland, New Zealand), and the 1,953 bp fragment containing the ColE1 origin and multiple cloning site purified and treated with Klenow enzyme (Roche) to give a blunt-ended fragment. A gene encoding resistance to erythromycin (Em) was isolated on a 1.6 kb fragment obtained after cutting pVA891 (Macrina et al., Gene 25:145-50, 1983) with ClaI and HindIII and treatment with Klenow to give blunt ends. The 1.6 kb Em fragment was ligated to the 1,953 bp pBS-SK+ fragment, transformed into E. coli TG1 (Gibson T J, Studies on the Epstein-Barr virus genome. Ph.D. Thesis, University of Cambridge, Cambridge, England, 1984), and plated on LB agar plates containing 200 μg/ml Em. Maintenance of α-complementation for blue/white color selection of recombinant pBEry1 clones was confirmed by growing E. coli colonies on agar plates containing IPTG and X-gal.

The resulting pBEry1 construct encoding the HN001 deoD purine nucleoside phosphorylase AQ1 gene was transformed into competent HN001 cells and grown anaerobically for 48 hrs at 37° C. on MRS lactobacilli agar (Difco, Detroit Mich.) containing 2.5 μg/ml Em. Em-resistant HN001 were checked for integration of the plasmid construct into the deoD gene by PCR using vector-specific (T3 or T7) and AQ1 internal fragment-specific primers.

Colonies giving PCR patterns consistent with the insertional inactivation of the endogenous HN001 deoD purine nucleoside phosphorylase AQ1 gene were assessed for increased resistance to UV irradiation. Briefly, single colonies of wild-type of AQ1-knockout HN001 strains were inoculated into 5 ml MRS, incubated aerobically overnight at 37° C., and 0.1 ml then used to inoculate a 10 ml MRS culture. Cultures were grown to log phase (i.e. an OD₆₀₀ of approximately 0.3) at 37° C., and then 5 ml of culture taken and cells collected by centrifugation. Cells were resuspended in 1 ml normal saline (0.9% NaCl), and 20 μl aliquots placed on sterile petri dishes. Petri dishes were then placed uncovered and inverted onto a standard laboratory transilluminator and exposed to UV light for 0, 20 or 30 seconds. Samples were transferred to 1 ml MRS media and grown for 2 hours at 37° C. in the dark. Following culture, samples were appropriately diluted and duplicate samples plated onto MRS plates, incubated anaerobically for 48 hours at 37° C. and colonies counted.

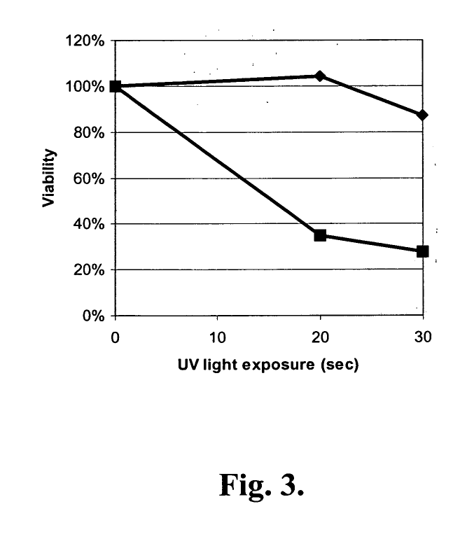

FIG. 3 shows the results of UV light exposure assay measuring relative viability in response to increasing doses of UV light for AQ1⁻ HN001 strain (♦) and wild-type HN001 (▪). Results indicate that the AQ1⁻ HN001 mutant strain showed enhanced survival to exposure to UV light compared to wild-type HN001. UV light exposure of 20 seconds appeared to have no effect on AQ1⁻ viability while the viability of wild-type HN001 cells had dropped to 34.7%. After 30 seconds UV light exposure, 86.9% of AQ1⁻ cells survived compared to only 27.9% for wild type. Therefore, removal of AQ1 gene expression led to enhanced survival of UV light exposure, indicating that AQ1 encodes the HN001 deoD purine nucleoside phosphorylase.

Purine nucleoside phosphorylase (EC 2.4.2.1) is involved in the purine biosynthesis and salvage pathways. Its role in maintaining intracellular guanosine pools suggests that it may be involved in resistance to a number of stress conditions including UV light exposure, as well as high salt, pH and temperature (Duwat et al, Int J Food Microbiol. 55:83-6, 2000). Applications for HN001 purine nucleoside phosphorylase AQ1 include:
methods of enhanced survival of industrial processes;
improved colonization of human intestinal environment; and
improved survival of multiple stress conditions.

### EXAMPLE 3

### Isolation and Characterization of GTP Pyrophosphokinase from L. Rhamnosus HN001

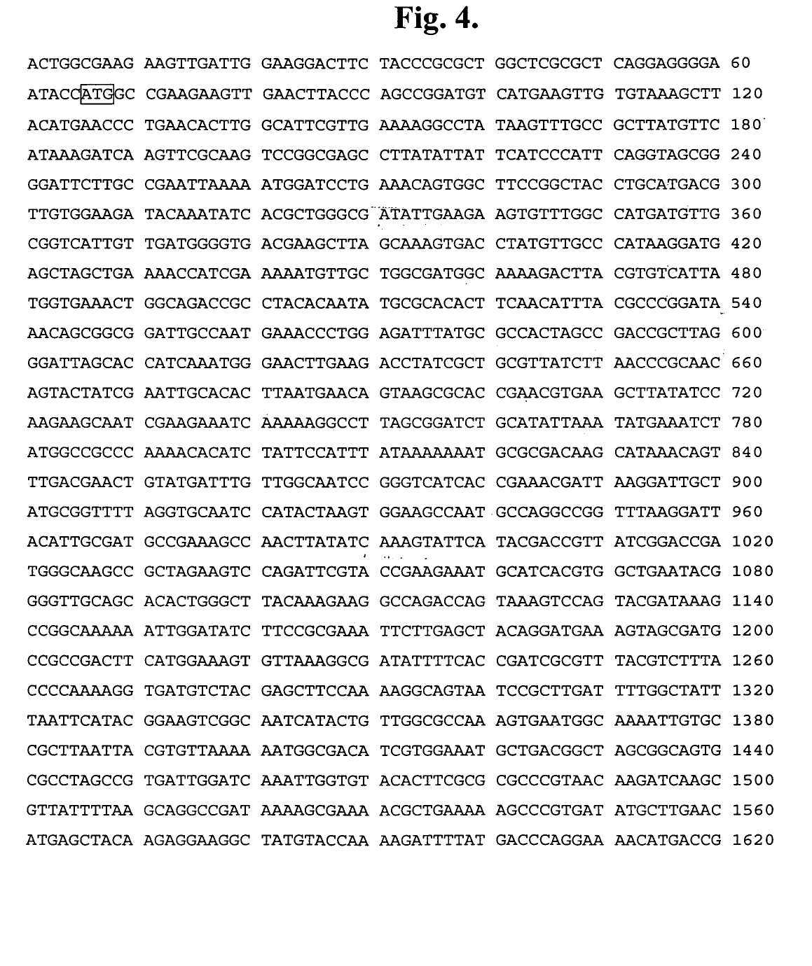

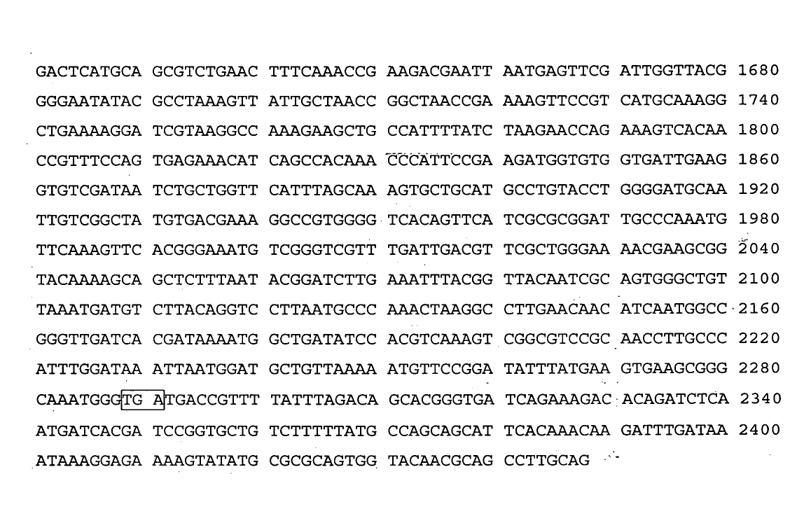

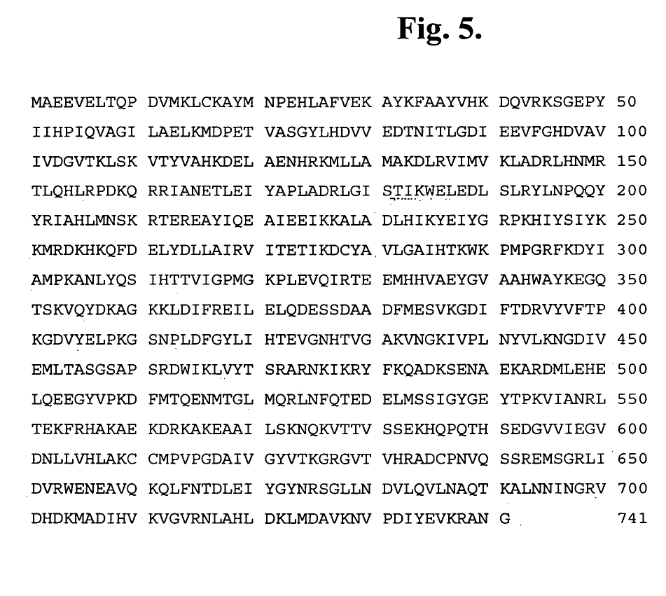

The full-length polynucleotide sequence of the relA GTP pyrophosphokinase gene AM1 from L. rhamnosus HN001 is given in SEQ ID NO: 79 and shown in FIG. 4 (with ATG initiation and translation stop codons boxed). The polypeptide sequence of AM1 is given in SEQ ID NO: 182 and shown in FIG. 5.

A 798 bp internal AM1 fragment was amplified by PCR using standard laboratory protocols. The nucleotide sequences of the oligonucleotide primers are given in SEQ ID NOS: 186 and 187. The fragments were cloned into the pBEry1 vector cut with SmaI, as described in Example 2. The resulting pBEry1 construct encoding the HN001 relA GTP pyrophosphokinase AM1 gene was transformed into competent HN001 cells and grown anaerobically for 48 hrs at 37° C. on MRS lactobacilli agar (Difco, Detroit Mich.) containing 2.5 μg/ml Em. Em-resistant HN001 were checked for integration of the plasmid construct into the relA gene by PCR using vector-specific (T3 or T7) and AM1 internal fragment-specific primers.

Colonies giving PCR patterns consistent with the insertional inactivation of the endogenous HN001 relA GTP pyrophosphokinase AM1 gene, were assessed for increased resistance to UV irradiation as described in Example 2.

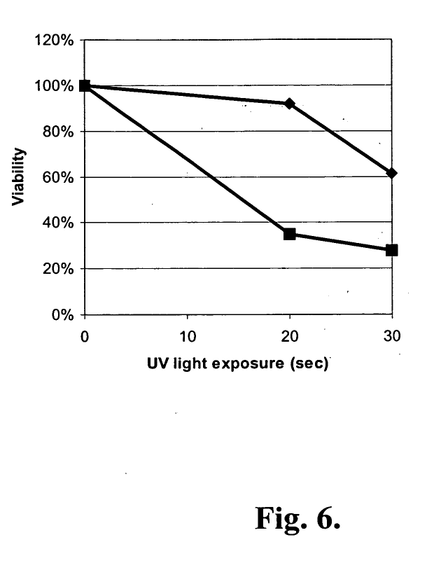

FIG. 6 shows the results of a UV light exposure assay measuring relative viability in response to increasing doses of UV light in AM1⁻ HN001 (♦) and wild-type HN001 (▪) strains. Results indicate that the AM1⁻ HN001 mutant strain showed enhanced survival to exposure to UV light compared to wild-type HN001. UV light exposure of 20 seconds appeared to have little effect on AM1⁻ cell viability (91.7%) while the viability of wild-type HN001 cells had dropped to 34.7%. After 30 seconds UV light exposure, 61.1% of AM1⁻ cells survived compared to only 27.9% for wild type. Therefore, removal of AM1 gene expression led to enhanced survival of UV light exposure, indicating that AM1 encodes the HN001 relA GTP pyrophosphokinase.

GTP pyrophosphokinase or (EC 2.7.6.5) produces guanosine 3′-diphosphate 5′-triphosphate, a marker of the “stringent response”, a regulatory state induced in bacteria by nutrient starvation and other environmental stresses (reviewed in Chatterji and Ojha, Curr Opin Microbiol. 4:160-5, 2001). Studies have indicated that suppression of GTP pyrophosphokinase relA gene expression improved the resistance to a number of stress conditions including UV light exposure, as well as high salt, pH and temperature, in Lactococcus lactis (Duwat et al, Int J. Food Microbiol. 55:83-6, 2000). Applications for HN001 GTP pyrophosphokinase AM1 include:
methods of enhanced survival of industrial processes;
improved colonization of human intestinal environment; and
improved survival of Lactobacilli to multiple stress conditions.

SEQ ID NOS: 1-187 are set out in the attached Sequence Listing. The codes for nucleotide sequences used in the attached Sequence Listing, including the symbol “n,” conform to WIPO Standard ST.25 (1998), Appendix 2, Table 1.

All references cited herein, including patent references and non-patent publications, are hereby incorporated by reference in their entireties.

While in the foregoing specification this invention has been described in relation to certain preferred embodiments, and many details have been set forth for purposes of illustration, it will be apparent to those skilled in the art that the invention is susceptible to additional embodiments and that certain of the details described herein may be varied considerably without departing from the basic principles of the invention.

1. An isolated polynucleotide comprising a sequence selected from the group consisting of: SEQ ID NO: 1-80.

2. An isolated polynucleotide comprising a sequence selected from the group consisting of:
(a) complements of SEQ ID NO: 1-80;
(b) reverse complements of SEQ ID NO: 1-80; and
(c) reverse sequences of SEQ ID NO: 1-80.

3. An isolated polynucleotide comprising a sequence selected from the group consisting of:
(a) sequences having at least 75%, identity to a sequence of SEQ ID NO: 1-80;
(b) sequences having at least 90% identity to a sequence of SEQ ID NO: 1-80; and
(c) sequences having at least 95% identity to a sequence of SEQ ID NO: 1-80, wherein the polynucleotide encodes a polypeptide having substantially the same functional properties as a polypeptide encoded by SEQ ID NO: 1-80.

4. An isolated polynucleotide comprising a sequence selected from the group consisting of:
(a) nucleotide sequences that are 200-mers of a sequence recited in SEQ ID NO: 1-80;
(b) nucleotide sequences that are 100-mers of a sequence recited in SEQ ID NO: 1-80;
(c) nucleotide sequences that are 40-mers of a sequence recited in SEQ ID NO: 1-80; and
(d) nucleotide sequences that are 20-mers of a sequence recited in SEQ ID NO: 1-80;

5. An isolated oligonucleotide probe or primer comprising at least 10 contiguous residues complementary to 10 contiguous residues of a nucleotide sequence recited in any one of claims 1-3.

6. A kit comprising a plurality of oligonucleotide probes or primers of claim 5.

7. A genetic construct comprising a polynucleotide of any one of claims 1-3.

8. A transgenic host cell comprising a genetic construct according to claim 7.

9. A genetic construct comprising, in the 5′-3′ direction:
(a) a gene promoter sequence; and
(b) a polynucleotide sequence comprising at least one of the following: (1) a polynucleotide coding for at least a functional portion of a polypeptide of SEQ ID NO: 81-183; and (2) a polynucleotide comprising a non-coding region of a polynucleotide of any one of claims 1-3.

10. The genetic construct of claim 9, wherein the polynucleotide is in a sense orientation.

11. The genetic construct of claim 9, wherein the polynucleotide is in an anti-sense orientation.

12. The genetic construct of claim 9, wherein the gene promoter sequence is functional in a prokaryote or eukaryote.

13. A transgenic host cell comprising a construct of claim 9.

14. A transgenic organism comprising a transgenic host cell according to claim 13, or progeny thereof.

15. The transgenic organism of claim 15, wherein the organism is selected from the group consisting of Lactobacillus species.

16. A method for modulating the activity of a polypeptide in an organism, comprising stably incorporating into the genome of the organism a polynucleotide of any one of claims 1-3.

17. The method of claim 16, wherein the organism is a microbe.

18. An isolated polypeptide comprising an amino acid sequence selected from the group consisting of: sequences recited in SEQ ID NO: 81-183.

19. An isolated polypeptide comprising an amino acid sequence selected from the group consisting of:
(a) sequences having at least 75% identity to a sequence of SEQ ID NO: 81-183;
(b) sequences having at least 90% identity to a sequence of SEQ ID NO: 81-183; and
(c) sequences having at least 95% identity to a sequence of SEQ ID NO: 81-183, wherein the polypeptide has substantially the same functional properties as a polypeptide of SEQ ID NO: 81-183.

20. An isolated polynucleotide that encodes a polypeptide of any one of claims 18 and 19.

21. An isolated polypeptide encoded by a polynucleotide of any one of claims 1-3.

22. A fusion protein comprising at least one polypeptide according to any one of claims 18 and 19.

23. A composition comprising a polypeptide according to any one of claims 18 and 19 and at least one component selected from the group consisting of: physiologically acceptable carriers and immunostimulants.

24. A composition comprising a polynucleotide according to any one of claims 1-3 and at least one component selected from the group consisting of: physiologically acceptable carriers and immunostimulants.

25. A method for treating a disorder in a mammal, comprising administering a composition according to claim 24.

26. A method for treating a disorder in a mammal, comprising administering a composition according to claim 25.
